(19) United States
(12) Patent Application Publication
(10) Pub. No.: US 2005/0009097 A1
(43) Pub. Date: Jan. 13, 2005

(54) Human engineered antibodies to Ep-CAM

(76) Inventors: Marc D. Better, Oakland, CA (US); Arnold H. Horwitz, San Leandro, CA (US)

Correspondence Address: Janet M. McNicholas, Ph.D.;McAndrews, Held & Malloy, Ltd., 34th Floor, 500 West Madison, Chicago, IL, 60661, US

(21) Appl. No.: 10/816,276

(22) Filed: Mar. 31, 2004

Related U.S. Application Data
(60) Provisional application No. 60/459,334, filed on Mar. 31, 2003.

(51) Int. Cl. G01N 33/53; C12P 21/04; C07K 16/40

(52) U.S. Cl. 435/7.1; 530/388.26

(57) ABSTRACT

Human engineered anti-Ep-CAM antibodies and various uses therefore are disclosed. These human engineered anti-Ep-CAM antibodies have high affinity binding to Ep-CAM with low immunogenicity.

### RELATED APPLICATIONS

This application claims the benefit under 35 U.S.C. § 119 of U.S. Provisional Application No. 60/459,334 filed Mar. 31, 2003, incorporated by reference herein.

### BACKGROUND OF THE INVENTION

Epithelial cell adhesion molecule (Ep-CAM) is a 40 kDa glycoprotein expressed on the basolateral surface of many, but not all, human epithelial cells and most human adenocarcinomas [Balzar et al., J. Mol. Med., 77:699-712 (1999)]. Ep-CAM, known by many other names including 17-1A antigen [Herlyn et al., Proc. Natl. Acad. Sci., 79:4761-4765(1979)], KSA [Perez and Walker, J Hematol., 142:3662-3667 (1989)], EGP [Strnad et al., Cancer Res., 49:314-317 (1989)], EGP40 [Simon et al., Proc. Natl. Acad. Sci., 87:2755-2759 (1990)] and GA733-2 [Szala et al., Proc. Natl. Acad. Sci., 87:3542-3546 (1990)], is a transmembrane protein comprised of 314 amino acids, of which 265 contribute to the extracellular domain.

Ep-CAM functions as a homophilic adhesion molecule that promotes relatively weak, flexible cell-cell interactions which appear to be important for epithelial tissue morphogenesis and embryonic development [Litvinov et al., Cancer Res., 54:1753-1759 (1994)]. Ep-CAM-mediated adhesions are blocked by monoclonal antibodies [Cirulli et al., J Cell Biol 140:1519-1534 (1998)], are calcium independent [Litvinov et al., 1994 Cancer Res. 54:1753-1759], and are mediated by interaction of the 26 amino acid cytoplasmic domain with the cytoskeleton via alpha-actinin [Balzar et al., Mol Cell Biol 18:4833-4843 (1998)].

Increased expression of Ep-CAM is associated with active proliferation of epithelial cells and a less differentiated phenotype [Litvinov et al., Am J Pathol 148:865-875 (1996)]. Epithelial cells with little or no Ep-CAM expression begin to express Ep-CAM during their transition to neoplasias [Zorzos et al., Eur Urol 28:251-254 (1995); High et al., J Oral pathol Med 25:10-13 (1996), Litvinov et al., Am J Pathol 148:865-875 (1996)]. Most adenocarcinomas, but no other tumor types, express Ep-CAM although the level of expression within a tumor can be heterogeneous due to shifts in cell phenotype. Some studies suggest that Ep-CAM expression may be associated with a reduction in metastasis [Takes et al., Arch Otolaryngol Head Neck Surg 123:412-419 (1997); Balzar et al., Mol Cell Biol 18:4833-4843 (1998)], but there also are reports to the contrary [Tandon et al., Cancer Res 50:3317-3321 (1990)].

Ep-CAM is an attractive target for immuno-therapy of adenocarcinomas including breast cancer, colorectal cancer (CRC), non-small cell lung cancer (NSCLC) and prostate cancer. The mouse anti-carcinoma antibody Br-1, first made and characterized by Colcher et al. where it was designated as B38.1 (See, e.g., described in U.S. Pat. No. 4,612,282), binds to Ep-CAM. However, the application of unmodified mouse monoclonal antibodies in the treatment of human diseases are problematic for several reasons. First, an immune response against the mouse antibodies may be mounted in the human body (human anti-murine antibody (HAMA) response). Second, the mouse antibodies may have a reduced half-life in the human circulatory system. Third, the mouse antibody effector domains may not efficiently trigger the human immune system.

Br-1 has previously been altered with the goal of generating a useful therapeutic reagent. A mouse-human chimeric antibody, called ING-1, containing the Br-1 mouse variable region domains and human constant region domains has been constructed (U.S. Pat. No. 5,576,184). As a therapeutic, the mouse-human chimeric ING-1 antibody has advantages over its all-mouse BR-1 counterpart due to the inclusion of a human Fc portion. While retaining identical binding affinity and selectivity to Ep-CAM, ING-1 has more potent effector activities than Br-1. These increased effector functions, include antibody-dependent cellular cytotoxicity (ADCC) and complement-dependent cytolysis (CDC).

The development of chimeric antibodies, such as the mouse-human chimeric ING-1 antibody, has provided the basis of what is now known as antibody genetic engineering or human-engineering. Mouse-human chimeric antibodies in general show the same specificity and affinity as the parental murine antibody and are capable of efficiently mediating ADCC and complement fixation in the human context. However, many chimeric versions of potential therapeutic murine antibodies have been evaluated in clinical trials and it has become clear that, while in some cases chimerization caused a total disappearance of the HAMA response, many chimeric antibodies remained immunogenic as a result of the presence of the murine variable regions.

Although anti-Ep-CAM antibodies have been developed, there is a still unmet need for antibodies of high affinity and low immunogenicity that target and inhibit or kill Ep-CAM-expressing tumor cells.

The immunogenicity of therapeutic antibodies is a significant problem and severely limits the widespread and repeated application of murine monoclonal antibodies (Mab) to treat many diseases. In addition to the development of chimeric antibodies, other engineering strategies have been adopted to circumvent the immunogenicity of potential therapeutic antibodies. Another way of reducing the immunogenicity of murine variable regions involves taking the genetic information from murine hypervariable regions or complementarity determining regions (hereinafter referred to as CDRs) and inserting them in place of the DNA encoding the CDRs of a human monoclonal antibody to generate a construct encoding a human antibody with murine CDRs. This technique is known as CDR grafting. See, e.g., Jones et al., Nature, 321, 522-525 (1986); Junghans et al., supra.

The technique of CDR grafting was originally described by Winter and colleagues at the Medical Research Council (MRC) (See, e.g., U.S. Pat. No. 5,225,539). Winter proposed an altered antibody or antigen-binding fragment where the variable domain has framework regions of a first immunoglobulin heavy or light chain and CDR regions of a second different immunoglobulin heavy or light chain. Winter's method results in variable domains in which both the heavy and light chains have been altered by CDR replacement, and requires that CDRs in the light or heavy variable domains of the engineered antibody be replaced by analogous CDRs from an antibody of different specificity.

However, as a result of the humanization of mouse monoclonal antibodies by CDR grafting, specific binding activity of the resulting humanized antibodies may be diminished or even completely abolished. For example, the binding affinity of the modified antibody described in Queen et al., supra, is reported to be reduced three-fold; in Co et al., supra, is reported to be reduced two-fold; and in Jones et al., supra, is reported to be reduced two- to three-fold. Other reports describe order-of-magnitude reductions in binding affinity. See, e.g., Tempest et al., Bio/Technology, 9 266-271 (1991); Verhoeyen et al., Science, 239, 1534-1536 (1988).

Queen et al. (U.S. Pat. Nos. 5,693,762, 5,693,761, 5,585,089, and 6,180,370), has proposed a method of producing humanized immunoglobulins having the CDRs and one or more additional amino acids from a donor immunoglobulin involved in antigen binding which are transferred to a framework region of an acceptor human immunoglobulin. Queen defines the donor as the non-human immunoglobulin providing the CDRs, and defines the acceptor as the human immunoglobulin providing the framework. Queen's method results in an humanized immunoglobulin having CDRs from a donor immunoglobulin (i.e., non-human) and variable region frameworks from a human acceptor immunoglobulin, in addition to one or more amino acids from the donor immunoglobulin outside the CDRs that replace the corresponding amino acids in the acceptor immunoglobulin variable region framework.

Adair et al. (U.S. Pat. No. 5,859,205) has also provided a method intended to transfer the binding site of an antibody into a different acceptor framework. Adair's method requires the design of a humanized antibody where the aim is to transfer the minimum number of mouse amino acids that would confer antigen binding onto a human antibody framework. Adair's method, as well as Queen et al, is widely applicable to the CDR-grafting of antibodies in general. The CDR-grafted antibody chains are designed starting from the basis of the acceptor sequence whereby as a first step donor residues are substituted for acceptor residues in the CDRs followed by additional non-CDR donor residues which contribute to antigen binding.

In addition, Carter et al. (U.S. Pat. Nos. 6,407,213 and 6,054,297) also provides a method which produces a humanized antibody variable domain comprising non-human CDR amino acid residues which bind an antigen incorporated into a human antibody variable domain. Carter's method incorporates steps which include transferring at least one CDR from a non-human, import sequence into a consensus human structure, after the entire corresponding human CDR has been removed. Import residues are defined as non-human residues which have the desired affinity and/or specificity. An integral step in Carter's approach to antibody engineering is the construction of computer graphics models of the import and humanized antibody to determine if the six complementarity-determining regions (CDRs) can be successfully transplanted from the import framework to a human one and to determine which framework residues from the import antibody, if any, need to be incorporated into the humanized antibody in order to maintain CDR conformation. These above-described techniques for humanizing murine variable domains are widely applicable to the CDR-grafting of antibodies in general with the added transfer of additional non-CDR residues from a non-human donor onto a human acceptor variable region framework.

In contrast to all of the above-described methods which involve CDR grafting typically with some framework changes, Studnicka et al. developed a method to modify any variable region of any antibody to reduce immunogenicity while maintaining antigen binding. The method permits starting with a variable region of any one species and modifying it with the residues of a variable region of any other species. Specifically, the method permits direct construction of a fully-active human engineered antibody using any non-human variable region sequence. Unlike all of the above-described techniques, Studnicka's method does not involve CDR grafting technology. Unlike all of the above-described techniques, Studnicka does not propose any transfer of non-human (e.g. murine) amino acids that would confer antigen binding onto a human antibody variable region framework. Studnicka's method does not employ any donor or acceptor sequences. Specifically, Studnicka's method does not employ a human acceptor heavy or light chain variable region modified by importing CDR and non-CDR amino acid residues from a non-human donor immunoglobulin. The technology is based on a unique residue-by-residue analysis to determine which amino acid residues, for example, along a non-human antibody variable region are candidates to be changed to human to reduce immunogenicity while preserving antibody binding. As such, the Studnicka method employs a rule-set based on the analysis of each amino acid position in the antibody variable region. The rule-set was developed from a 2 parameter analysis that compared the benefit of reducing immunogenicity to the risk of adversely affecting specific antigen binding or proper antibody folding.

### SUMMARY OF THE INVENTION

The present invention provides human engineered anti-Ep-CAM antibodies. Preferred antibodies according to the invention bind to the epitope of human Ep-CAM bound by the mouse-human chimeric antibody ING-1 as produced by cell line HB9812 deposited with the ATCC. These preferred antibodies have low immunogenicity when administered to humans. Preferred antibodies according to the invention bind Ep-CAM with high affinity (about 1 to 5 nM or stronger) and have low immunogenicity. In preferred embodiments, the antibody is active in assays of antibody dependent cellular cytotoxicity (ADCC) and/or in assays of complement mediated cytotoxicity (CDC). Preferred antibodies according to the invention inhibit metastasis of cancer cells when tested in metastatic disease animal models.

The human engineered anti-Ep-CAM antibody may have a heavy chain variable region comprising the amino acid sequence of SEQ ID NO: 19 or SEQ ID NO: 21 and/or a light chain variable region comprising the amino acid sequence of SEQ ID NO: 6, SEQ ID NO: 8, SEQ ID NO: 35, SEQ ID NO: 37, SEQ ID NO: 39, SEQ ID NO: 41, SEQ ID NO: 43, or SEQ ID NO: 45. In other embodiments, the antibody comprises a variable region amino acid sequence modified to include one or more additional low risk changes and/or to include one or more additional moderate risk changes. Such variants will normally have a binding affinity for human Ep-CAM which is similar to that of the mouse-human chimeric antibody ING-1 as produced by cell line HB9812.

In preferred embodiments, the human engineered ING-1 antibody includes a light chain variable region comprising the variable region amino acid sequence of SEQ ID NO: 6 and/or a heavy chain comprising the variable region amino acid sequence of SEQ ID NO: 19 and/or amino acid sequence variants thereof. In a most preferred embodiment, the human engineered ING-1 antibody comprises a light chain variable region that is SEQ ID NO: 6 and a heavy chain variable region that is SEQ ID NO: 19.

As described herein, it has been possible to reengineer a high affinity murine antibody with specificity for the human Ep-CAM antigen and produce high affinity, low immunogenicity human engineered antibodies.

Various forms of the human engineered antibody are contemplated herein. For example, the anti-Ep-CAM antibody may be a full length antibody (e.g., having a human immunoglobulin constant region) or an antibody fragment (e.g., a F(ab′)₂ Fab, Fv, scFv, SCA). Furthermore, the antibody may be labeled with a detectable label, immobilized on a solid phase and/or conjugated with a heterologous compound (such as a cytotoxic agent).

Diagnostic and therapeutic uses for human engineered anti-Ep-CAM antibodies are contemplated. In one diagnostic application, the invention provides a method for determining the presence of Ep-CAM protein comprising exposing a sample suspected of containing the Ep-CAM protein to the anti-Ep-CAM antibody and determining binding of the antibody to the sample. For this use, the invention provides a kit comprising the antibody and instructions for using the antibody to detect the Ep-CAM protein.

The invention also provides a composition comprising the human engineered anti-Ep-CAM antibody and a pharmaceutically acceptable carrier or diluent. This composition for therapeutic use is sterile and may be lyophilized. The invention further provides a method for treating a mammal suffering from Ep-CAM mediated disease, disorder or condition, comprising administering a pharmaceutically effective amount of the human engineered anti-Ep-CAM antibody to the mammal. The invention provides a human engineered anti-Ep-CAM antibody for use in therapy. The invention also provides a use of an anti-Ep-CAM antibody in the manufacture of a medicament for the treatment of a mammal with an Ep-CAM mediated disease, disorder or condition. An Ep-CAM mediated disease disorder or condition includes a carcinoma and/or metastasis of a cancer cell. Carcinomas include adenocarcinomas, for example, of the breast, lung, prostate, gastrointestinal tract (e.g., colon/large intestine, small intestine, rectum, pancreas, stomach, esophagus) ovary, cervix, vagina, kidney, liver, bladder, bile duct, gallbladder, thyroid, endometrium, other organ or tissue. For such therapeutic uses, other chemotherapeutic agents, including, for example, immunosuppressive agents may be co-administered to the mammal either before, after, or simultaneously with, the human engineered anti-Ep-CAM antibody. The additional agent to be co-administered with or conjugated to the human engineered anti-Ep-CAM antibody may be a cytotoxic agent, a non-cytotoxic agent or an agent co-administered or conjugated that may be activated to be a cytotoxic agent.

The invention provides a method of killing or inhibiting the growth of an Ep-CAM expressing cancer cell comprising contacting the cell with an amount of the antibody effective to kill or inhibit the cell. The invention also provides a method of inhibiting metastasis of Ep-CAM expressing cancer cells comprising administering an amount of the antibody effective to inhibit the metastasis of the cells. For such methods, other agents such as a cytotoxic, non-cytotoxic, or chemotherapeutic agent, may be used to contact the cells, for example, by administration either before, after, or simultaneously with, or by conjugation to, the human engineered anti-Ep-CAM antibody.

The invention further provides isolated nucleic acid encoding the antibody; a vector comprising that nucleic acid, optionally operably linked to control sequences recognized by a host cell transformed with the vector; a host cell comprising that vector; a process for producing the antibody comprising culturing the host cell so that the nucleic acid is expressed and, optionally, recovering the antibody from the host cell culture (e.g., from the host cell culture medium).

As discussed herein, an exemplary anti-Ep-CAM antibody, murine Br-1, was modified to be less immunogenic in humans based on the human engineering method of Studnicka et al. In a preferred embodiment, 13 surface exposed amino acid residues of the anti-Ep-CAM antibody heavy chain variable region and 6 in the anti-Ep-CAM light chain region were modified to human residues in positions determined to be unlikely to adversely effect either antigen binding or protein folding, while reducing its immunogenicity with respect to a human environment. Synthetic genes containing modified heavy and/or light chain variable regions were constructed and linked to human γ heavy chain and/or kappa light chain constant regions. Any human heavy chain and light chain constant regions may be used in combination with the human engineered antibody variable regions. The human heavy and light chain genes were introduced into mammalian cells and the resultant recombinant immunoglobulin products were obtained and characterized.

### BRIEF DESCRIPTION OF THE DRAWINGS

These and other features, aspects, and advantages of the present invention will become better understood with reference to the following description, appended claims, and accompanying drawings where:

FIG. 1 depicts a construction map for vector pING1928.

FIG. 2 depicts a construction map for vector pING1931.

FIG. 3 depicts construction maps for vector pING1932 and pING1932R.

FIG. 4 depicts a construction map for vector pING1933.

FIG. 5 shows the amino acid changes (underlined) made during human engineering of the ING-1 light chain variable region.

FIG. 6 depicts a construction map for vector pING1936.

FIG. 7 shows the amino acid changes (underlined) made during human engineering of the ING-1 heavy chain variable region.

FIG. 8 depicts a construction map for vector pING1937.

FIG. 9 depicts a construction map for vector pING1959.

FIG. 10 depicts a construction map for vector pING1957.

FIG. 11 depicts a construction map for vector pING1963.

FIG. 12 depicts a construction map for vector pING1964.

FIG. 13 depicts a construction map for vector pING1965.

FIG. 14 shows the structure of vector pING1964 linearized with Not1.

FIG. 15 shows competition binding results for human engineered low risk ING-1.

FIG. 16 shows competition binding results for human engineered low plus moderate risk ING-1.

FIG. 17 shows competition binding results for ING-1 with combinations of light and heavy chains modified at either low or low plus moderate risk positions.

FIG. 18A depicts construct strategy for proline changes in human engineered (low risk) ING-1, and FIG. 18B shows competition binding results for ING-1 light chain with single or pair combinations of moderate risk proline changes.

FIG. 19 depicts a construction map for vector pING1954.

FIG. 20 shows a direct binding ELISA for human engineered (low risk) ING-1 with soluble Ep-CAM.

### DETAILED DESCRIPTION

Human engineered antibodies directed to Ep-CAM are provided according to the invention. Treatment of Ep-CAM-related diseases, disorders or conditions is made possible with such human engineered antibodies.

Treatment refers to both therapeutic treatment and prophylactic or preventative measures. Those in need of treatment include those already with the disorder as well as those in which disease, condition or the disorder is to be prevented.

Mammal for purposes of treatment refers to any animal classified as a mammal, including humans, domestic and farm animals, and zoo, sports, or pet animals, such as dogs, horses, cats, cows, etc. Preferably, the mammal is human.

The term antibody is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

Antibody fragments comprise a portion of a full length antibody, generally the antigen binding or variable region thereof. Examples of antibody fragments include Fab, Fab′, F(ab′), and Fv/ScFv fragments; diabodies; linear antibodies; single-chain antibody molecules; and multispecific antibodies formed from antibody fragments.

The term monoclonal antibody refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier monoclonal indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256: 495 (1975), or may be made by recombinant DNA methods (See, e.g., U.S. Pat. No. 4,816,567). The monoclonal antibodies may also be isolated from phage antibody libraries, for example, using the techniques described in Clackson et al., Nature 352: 624-628 (1991) and Marks et al., J. Mol. Biol. 222: 581-597 (1991).

The monoclonal antibodies herein specifically include chimeric antibodies (immunoglobulins) in which a portion of the heavy and/or light chain is identical with or homologous to corresponding sequences in antibodies derived from one antibody sequence, including those derived from a particular species or belonging to a particular antibody class or subclass, while the remainder of the chain(s) is identical with or homologous to corresponding sequences in antibodies derived from another antibody sequence, including those derived from another species or belonging to another antibody class or subclass, as well as fragments of such antibodies, so long as they exhibit the desired biological activity [see, e.g., U.S. Pat. No. 4,816,567; and Morrison et al., Proc. Natl. Acad Sci. USA 81: 6851-6855 (1984)].

The term hypervariable region refers to the amino acid residues of an antibody which are responsible for antigen-binding. The hypervariable region comprises amino acid residues from a complementarity determining region or CDR [i.e., residues 24-34 (L1), 50-56 (L2) and 89-97 (L3) in the light chain variable domain and 31-35 (H1), 50-65 (H2) and 95-102 (H3) in the heavy chain variable domain as described by Kabat et al., Sequences of Proteins of Immunological Interest, 5th Ed. Public Health Service, National Institutes of Health, Bethesda, Md. (1991)] and/or those residues from a hypervariable loop (i.e., residues 26-32 (L1), 50-52 (L2) and 91-96 (L3) in the light chain variable domain and 26-32 (H1), 53-55 (H2) and 96-101 (H3) in the heavy chain variable domain as described by [Chothia et al., J. Mol. Biol. 196: 901-917 (1987)]. Framework or FR residues are those variable domain residues other than the hypervariable region residues.

Humanized forms of non-human (e.g., murine) antibodies are chimeric antibodies which contain minimal sequence derived from non-human immunoglobulin. Specifically, humanized antibodies are human immunoglobulins (recipient antibody) in which hypervariable region residues of the recipient are replaced by hypervariable region residues from a non-human species (donor antibody) such as mouse, rat, rabbit or nonhuman primate having the desired specificity, affinity, and capacity (i.e., CDR grafted antibodies). In some instances, in addition, framework region (FR) residues of the human immunoglobulin are replaced by corresponding non-human residues. Furthermore, humanized antibodies may comprise residues which are not found in the recipient antibody or in the donor antibody. These additional modifications are made to further refine antibody performance. Thus, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see, e.g., Jones et al., Nature 321: 522-525 (1986); Reichmann et al., Nature 332: 323-329 (1988); and Presta, Curr. Op. Struct. Biol. 2: 593-596 (1992).

Single-chain Fv or sFv antibody fragments comprise variable regions of the heavy chain (V_{H}) and the light chain (V_{L}) of antibody, wherein these regions are present in a single polypeptide chain. Generally, the Fv polypeptide further comprises a polypeptide linker between the V_{H} and V_{L} domains which enables the sFv to form the desired structure for antigen binding. For a review of sFv see, e.g., Pluckthun in The Pharmacology of Monoclonal Antibodies, vol. 113, Rosenburg and Moore eds. Springer-Verlag, New York, pp. 269-315 (1994).

The term diabodies refers to small antibody fragments with two antigen-binding sites, which fragments comprise a heavy chain variable region (V_{H}) connected to a light chain variable region (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90: 6444-6448 (1993).

Linear antibodies refer to the antibodies described in Zapata et al. Protein Eng. 8(10): 1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific.

An isolated antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% homogeneity by SDS-PAGE under reducing or nonreducing conditions using Coomassie blue or, preferably, silver stain or (2) to a degree sufficient to obtain at least 15 residues of N-terminal or internal amino acid sequence by use of a spinning cup sequenator. Isolated antibody includes the antibody in situ within recombinant cells since at least one component of the antibody's natural environment will not be present. Ordinarily, however, isolated antibody will be prepared by at least one purification step.

The term epitope tagged refers to the anti-Ep-CAM antibody fused to an epitope tag. The epitope tag polypeptide has enough residues to provide an epitope against which an antibody there against can be made, yet is short enough such that it does not interfere with activity of the Ep-CAM antibody. The epitope tag preferably is sufficiently unique so that the antibody there against does not substantially cross-react with other epitopes. Suitable tag polypeptides generally have at least 6 amino acid residues and usually between about 8-50 amino acid residues (preferably between about 9-30 residues). Examples include the flu HA tag polypeptide and its antibody 12CA5 [Field et al., Mol. Cell. Biol. 8: 2159-2165 (1988)]; the c-myc tag and the 8F9, 3C7, 6E10, G4, B7 and 9E10 antibodies thereto [Evan et al., Mol. Cell. Biol. 5(12): 3610-3616 (1985)]; and the Herpes Simplex virus glycoprotein D (gD) tag and its antibody [Paborsky et al., Protein Engineering 3(6): 547-553 (1990)]. In certain embodiments, the epitope tag is a salvage receptor binding epitope. As used herein, the term salvage receptor binding epitope refers to an epitope of the Fc region of an IgG molecule (e.g., IgG₁, IgG₂, IgG₃, or IgG₄) that is responsible for increasing the in vivo serum half-life of the IgG molecule.

A cytotoxic agent refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin or synthetic toxins, or fragments thereof. A non-cytotoxic agent refers to a substance that does not inhibit or prevent the function of cells and/or does not cause destruction of cells. A non-cytotoxic agent may include an agent that can be activated to be cytotoxic. A non-cytotoxic agent may include a bead, liposome, matrix or particle (see, e.g., U.S. Patent Publications 2003/0028071 and 2003/0032995 which are incorporated by reference herein). Such agents may be conjugated, coupled, linked or associated with a human engineered anti-Ep-CAM antibody according to the invention.

A chemotherapeutic agent is a chemical compound useful in the treatment of cancer. Examples of chemotherapeutic agents include Adriamycin, Doxorubicin, 5-Fluorouracil, Folinic acid, Cytosine arabinoside (Ara-C), Cyclophosphamide, Thiotepa, Taxotere (docetaxel), Busulfan, Cytoxin, Taxol, Methotrexate, Cisplatin, Dolastatin, Auristatin, CPT-11, (Irinotecan, CAMPTOSAR), Gemcitabine (Gemzar®) Melphalan, Vinblastine, Bleomycin, Etoposide, Ifosfamide, Mitomycin C, Mitoxantrone, Vincreistine, Vinorelbine, Carboplatin, Teniposide, Daunomycin, Carminomycin, Aminopterin, Dactinomycin, Mitomycins, Esperamicins (see, e.g., U.S. Pat. No. 4,675,187), Melphalan and other related nitrogen mustards.

Prodrug refers to a precursor or derivative form of a pharmaceutically active substance that is less cytotoxic or non-cytotoxic to tumor cells compared to the parent drug and is capable of being enzymatically activated or converted into an active or the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). Prodrugs include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use herein include, but are not limited to, those chemotherapeutic agents described above.

Label refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

Solid phase refers to a non-aqueous matrix to which the antibody of the present invention can adhere. Examples of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column). This term also includes a discontinuous solid phase of discrete particles, such as those described in U.S. Pat. No. 4,275,149.

A liposome is a small vesicle composed of various types of lipids, phospholipids and/or surfactant which is useful for delivery of a drug (such as the anti-Ep-CAM antibodies disclosed herein and, optionally, a chemotherapeutic agent) to a mammal. The components of the liposome are commonly arranged in a bilayer formation, similar to the lipid arrangement of biological membranes.

An “isolated” nucleic acid molecule or “isolated” nucleic acid sequence is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

Expression control sequences refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Cell, cell line, and cell culture are often used interchangeably and all such designations herein include progeny. Transformants and transformed cells include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

A. Antibody Preparation

A method for human engineering a nonhuman Ep-CAM antibody is described in the Examples below. In order to human engineer an anti-Ep-CAM antibody, the nonhuman antibody starting material is prepared. Exemplary techniques for generating such antibodies will be described in the following sections.

(1) Antigen Preparation.

The Ep-CAM antigen to be used for production of antibodies may be a soluble form of the Ep-CAM antigen, as described in Example 7 below or other fragment of Ep-CAM (e.g. an Ep-CAM fragment comprising the epitope recognized by a mouse-human chimeric ING-1 antibody as produced by cell line HB9812 as deposited with the ATCC. Alternatively, cells expressing Ep-CAM at their cell surface can be used to generate antibodies. Such cells can be transformed to express Ep-CAM or may be other naturally occurring cells (e.g. HT-29 cells as described in Example 6 below). Other forms of Ep-CAM useful for generating antibodies will be apparent to those skilled in the art.

(2) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

(3) Monoclonal Antibodies

Monoclonal antibodies may be made using the hybridoma method first described by Kohler et al., Nature, 256: 495 (1975), or may be made by recombinant DNA methods (U.S. Pat. No. 4,816,567).

In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp.59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOP-21 and M.C.-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107: 220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp.59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

(4) Human Engineering and Amino Acid Sequence Variants

Example 1 below describes methods for the human engineering of an anti-Ep-CAM antibody. In certain embodiments, it may be desirable to generate amino acid sequence variants of the human engineered antibody, particularly where these improve the binding affinity or other biological properties of the human engineered antibody.

Amino acid sequence variants of human engineering anti-Ep-CAM antibody are prepared by introducing appropriate nucleotide changes into the human engineered anti-Ep-CAM antibody DNA, or by peptide synthesis. Such variants include, for example, deletions from, and/or insertions into and/or substitutions of, residues within the amino acid sequences shown for the human engineered anti-Ep-CAM antibodies (e.g. as in SEQ ID NO: 6). Any combination of deletion, insertion, and substitution is made to arrive at the final construct, provided that the final construct possesses the desired characteristics. The amino acid changes also may alter post-translational processes of the humanized anti-Ep-CAM antibody, such as changing the number or position of glycosylation sites.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include human engineered anti-Ep-CAM antibody with an N-terminal methionyl residue or the antibody fused to an epitope tag. Other insertional variants of the human engineered anti-Ep-CAM antibody molecule include the fusion to the N- or C-terminus of human engineered anti-Ep-CAM antibody of an enzyme or a polypeptide which increases the serum half-life of the antibody (See below).

Another type of variant is an amino acid substitution variant. These variants have at least one amino acid residue in the human engineered anti-Ep-CAM antibody molecule removed and a different residue inserted in its place. The sites of greatest interest for substitutional mutagenesis include the hypervariable loops, but FR alterations are also contemplated. Hypervariable region residues or FR residues involved in antigen binding are generally substituted in a relatively conservative manner. Such conservative substitutions are shown below under the heading of preferred substitutions. If such substitutions result in a change in biological activity, then more substantial changes, denominated exemplary substitutions as shown below or as further described below in reference to amino acid classes, are introduced and the products screened.

TABLE I

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; | leu |
| | phe; | |
| Leu (L) | ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic: trp, tyr, phe.

Non-conservative substitutions will entail exchanging a member of one of these classes for another class. Any cysteine residue not involved in maintaining the proper conformation of the human engineered anti-EpCam antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of human engineered anti-Ep-CAM antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of human engineered anti-Ep-CAM antibody.

Ordinarily, amino acid sequence variants of the human engineered anti-Ep-CAM antibody will have an amino acid sequence having at least 75% amino acid sequence identity with the original human engineered antibody amino acid sequences of either the heavy or the light chain (e.g., as in SEQ ID NO: 19 or 6) more preferably at least 80%, more preferably at least 85%, more preferably at least 90%, and most preferably at least 95%, including for example, 80%, 81%, 82%, 83%, 84%, 85%, 86%, 87%, 88%, 89%, 90%, 91%, 92%, 93%, 94%, 95%, 96%, 97%, 98%, 99%, and 100%. Identity or homology with respect to this sequence is defined herein as the percentage of amino acid residues in the candidate sequence that are identical with the human engineered anti-Ep-CAM residues, after aligning the sequences and introducing gaps, if necessary, to achieve the maximum percent sequence identity, and not considering any conservative substitutions (as defined in Table I above) as part of the sequence identity. None of N-terminal, C-terminal, or internal extensions, deletions, or insertions into the antibody sequence shall be construed as affecting sequence identity or homology. Thus, sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences, or along a predetermined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences.

(5) Screening for Biological Properties

Antibodies having the characteristics identified herein as being desirable in a human engineered anti-Ep-CAM antibody are screened for.

To screen for antibodies which bind to the epitope on Ep-CAM bound by an antibody of interest (e.g., those which block binding of the mouse-human chimeric ING-1 antibody to Ep-CAM), a routine cross-blocking assay such as that described in Antibodies, A Laboratory Manual, Cold Spring Harbor Laboratory, Ed Harlow and David Lane (1988), can be performed. Competition binding assays are described in Example 6 below. Alternatively, epitope mapping, e.g. as described in Champe et al., J. Biol. Chem. 270: 1388-1394 (1995), can be performed to determine whether the antibody binds an epitope of interest.

Antibody affinities (e.g. for human Ep-CAM may be determined by saturation binding using HT-29 cells as described in Example 6 below. Preferred human engineered antibodies are those which bind human Ep-CAM with a K_{d} value of no more than about 1×10⁻⁷M; preferably no more than about 1×10⁻⁸M; more preferably no more than about 1×10⁻⁹M; and most preferably no more than about 1×10⁻¹⁰M. It is also desirable to select human engineered antibodies which have beneficial ADCC and/or CDC properties as described in Example 9 below.

(6) Antibody Fragments

In certain embodiments, the human engineered Ep-CAM antibody is an antibody fragment. Various techniques have been developed for the production of antibody fragments. Traditionally, these fragments were derived via proteolytic digestion of intact antibodies (See, e.g., Morimoto et al., Journal of Biochemical and Biophysical Methods 24: 107-117 (1992) and Brennan et al., Science 229: 81 (1985)). However, these fragments can now be produced directly by recombinant host cells. In breakthrough work published in 1988, Better et al., Science 240: 1041-1043 (1988) first achieved secretion of functional antibody fragments from bacteria (see, e.g., Better et al., Skerra et al. Science 240: 1038-1041 (1988)). A multiplicity of antibody fragments can be made in bacteria, for example, Fab′-SH fragments can be recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner.

(7) Multispecific Antibodies

In some embodiments, it may be desirable to generate multispecific (e.g. bispecific) human engineered Ep-CAM antibodies having binding specificities for at least two different epitopes. Exemplary bispecific antibodies may bind to two different epitopes of the Ep-CAM protein. Alternatively, an anti-Ep-CAM arm may be combined with an arm which binds to a triggering molecule on a leukocyte such as a T-cell receptor molecule (e.g., CD2 or CD3), or Fc receptors for IgG (FcγR), such as FcγRI (CD64), FcγRII (CD32) and FcγRIII (CD16) so as to focus cellular defense mechanisms to the Ep-CAM-expressing cell. Bispecific antibodies may also be used to localize cytotoxic agents to cells which express Ep-CAM. These antibodies possess an EpCam-binding arm and an arm which binds the cytotoxic agent (e.g., saporin, anti-interferon-alpha, vinca alkaloid, ricin A chain, methotrexate or radioactive isotope hapten). Bispecific antibodies can be prepared as full length antibodies or antibody fragments (e.g., F(ab′)₂ bispecific antibodies).

According to another approach for making bispecific antibodies, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g., tyrosine or tryptophan). Compensatory cavities of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers (see, e.g., WO96/27011).

Bispecific antibodies include cross-linked or heteroconjugate antibodies. For example, one of the antibodies in the heteroconjugate can be coupled to avidin, the other to biotin. Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′-SH fragments recovered from E. coli, can be chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the HER2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers [Kostelny et al., J. Immunol. 148(5): 1547-1553 (1992)]. The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The diabody technology described by Hollinger et al., Proc. Natl. Acad. Sci. USA 90: 6444-6448 (1993) has provided an alternative mechanism for making bispecific antibody fragments. The fragments comprise a heavy chain variable region (V_{H}) connected to a light-chain variable region (V_{L}) by a linker which is too short to allow pairing between the two domains on the same chain. Accordingly, the V_{H} and V_{L} domains of one fragment are forced to pair with the complementary V_{L} and V₄ domains of another fragment, thereby forming two antigen-binding sites. Another strategy for making bispecific antibody fragments by the use of single-chain Fv (sFv) dimers has also been reported. See Gruber et al., J. Immunol. 152: 5368 (1994). Alternatively, the bispecific antibody may be a linear antibody produced as described in Zapata et al. Protein Eng. 8(10): 1057-1062 (1995).

Antibodies with more than two valencies are contemplated. For example, trispecific antibodies can be prepared. Tutt et al., J. Immunol. 147: 60 (1991).

(8) Other Modifications

Other modifications of the human engineered anti-Ep-CAM antibody are contemplated. For example, it may be desirable to modify the antibody of the invention with respect to effector function, so as to enhance the effectiveness of the antibody in treating cancer, for example. For example cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176: 1191-1195 (1992) and Shopes, B. J. Immunol. 148: 2918-2922 (1992). Homodimeric antibodies with enhanced anti-tumor activity may also be prepared using heterobifunctional cross-linkers as described in Wolff et al., Cancer Research 53: 2560-2565 (1993). Alternatively, an antibody can be engineered which has dual Fc regions and may thereby have enhanced complement lysis and ADCC capabilities. See Stevenson et al., Anti-Cancer Drug Design 3: 219-230 (1989).

The invention also pertains to immunoconjugates comprising the antibody described herein conjugated to a cytotoxic agent such as a chemotherapeutic agent, toxin (e.g., an enzymatically active toxin of bacterial, fungal, plant or animal origin, or fragments thereof), or a radioactive isotope (i.e., a radioconjugate).

Chemotherapeutic agents useful in the generation of such immunoconjugates have been described above. Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, barley ribosome inactivating protein (BRIP), mitogellin, restrictocin, phenomycin, enomycin and the tricothecenes. A variety of radionuclides are available for the production of radioconjugated anti-Ep-CAM antibodies. Examples include ²¹²Bi, ¹³¹I, ¹³¹In, ⁹⁰Y and ¹⁸⁶Re.

Conjugates of the antibody and cytotoxic agent are made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionuclide to the antibody (see, e.g., WO94/11026).

In another embodiment, the antibody may be conjugated to a receptor (such streptavidin) for utilization in tumor pretargeting wherein the antibody-receptor conjugate is administered to the patient, followed by removal of unbound conjugate from the circulation using a clearing agent and then administration of a ligand (e.g., avidin) which is conjugated to a cytotoxic agent (e.g., a radionuclide).

The Ep-CAM antibodies disclosed herein may also be formulated as immunoliposomes. Liposomes containing the antibody are prepared by methods known in the art, such as described in Epstein et al., Proc. Natl. Acad. Sci. USA 82: 3688 (1985); Hwang et al., Proc. Natl. Acad. Sci. USA 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257: 286-288 (1982) via a disulfide interchange reaction. A chemotherapeutic agent (such as Doxorubicin) is optionally contained within the liposome [see, e.g., Gabizon et al., J. National Cancer Inst. 81(19): 1484 (1989)].

Human engineered antibodies of the present invention may also be used in ADEPT by conjugating the antibody to a prodrug-activating enzyme which converts a prodrug (e.g., a peptidyl chemotherapeutic agent, See WO81/01145) to an active anti-cancer drug. See, for example, WO88/07378 and U.S. Pat. No. 4,975,278.

The enzyme component of the immunoconjugate useful for ADEPT includes any enzyme capable of acting on a prodrug in such a way so as to covert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as abzymes, can be used to convert the prodrugs of the invention into free active drugs (See, e.g., Massey, Nature 328: 457-458 (1987)). Antibody-abzyme conjugates can be prepared as described herein for delivery of the abzyme to a tumor cell population.

The enzymes of this invention can be covalently bound to the anti-Ep-CAM antibodies by techniques well known in the art such as the use of the heterobifunctional crosslinking reagents discussed above. Alternatively, fusion proteins comprising at least the antigen binding region of an antibody of the invention linked to at least a functionally active portion of an enzyme of the invention can be constructed using recombinant DNA techniques well known in the art (See, e.g., Neuberger et al., Nature 312: 604-608 (1984)).

In certain embodiments of the invention, it may be desirable to use an antibody fragment, rather than an intact antibody, to increase tumor penetration, for example. In this case, it may be desirable to modify the antibody fragment in order to increase its serum half-life, for example, adding molecules such as PEG to antibody fragments to increase the half-life. This may be achieved, for example, by incorporation of a salvage receptor binding epitope into the antibody fragment (e.g., by mutation of the appropriate region in the antibody fragment or by incorporating the epitope into a peptide tag that is then fused to the antibody fragment at either end or in the middle, e.g., by DNA or peptide synthesis) (see, e.g., WO96/32478).

Covalent modifications of the human engineered Ep-CAM antibody are also included within the scope of this invention. They may be made by chemical synthesis or by enzymatic or chemical cleavage of the antibody, if applicable. Other types of covalent modifications of the antibody are introduced into the molecule by reacting targeted amino acid residues of the antibody with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, .alpha.-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing .alpha.-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay.

Carboxyl side groups (aspartyl or glutamyl) are selectively modified by reaction with carbodiimides (R—N.dbd.C.dbd.N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation of proline and lysine, phosphorylation of hydroxyl groups of seryl or threonyl residues, methylation of the .alpha.-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO87/05330 published 11 Sep. 1987, and in Aplin and Wriston, CRC Crit. Rev. Biochem., pp. 259-306 (1981).

Removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Hakimuddin, et al. Arch. Biochem. Biophys. 259: 52 (1987) and by Edge et al. Anal. Biochem., 118: 131 (1981). Enzymatic cleavage of carbohydrate moieties on antibodies can be achieved by the use of a variety of endo- and exo-glycosidases as described by Thotakura et al. Meth. Enzymol. 138: 350 (1987).

Another type of covalent modification of the antibody comprises linking the antibody to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337.

B. Vectors, Host Cells and Recombinant Methods

The invention also provides isolated nucleic acid encoding human engineered anti-Ep-CAM antibodies, vectors and host cells comprising the nucleic acids, and recombinant techniques for the production of the antibodies.

For recombinant production of the antibody, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

(1) Signal Sequence Component

The anti-Ep-CAM antibody of this invention may be produced recombinantly not only directly, but also as a fusion polypeptide with a heterologous polypeptide, which is preferably a signal sequence or other polypeptide having a specific cleavage site at the N-terminus of the mature protein or polypeptide. The signal sequence selected preferably is one that is recognized and processed (i.e., cleaved by a signal peptidase) by the host cell. If prokaryotic host cells do not recognize and process the native anti-Ep-CAM antibody signal sequence, the signal sequence may be substituted by a signal sequence selected, for example, from the group of the pectate lyase (e.g., pelB) alkaline phosphatase, penicillinase, lpp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, or the signal described in WO90/13646. In mammalian cell expression, mammalian signal sequences as well as viral secretory leaders, for example, the herpes simplex gD signal, are available.

The DNA for such precursor region is ligated in reading frame to DNA encoding the anti-Ep-CAM antibody.

(2) Origin of Replication Component

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 [plasmid origin is suitable for yeast, and various viral origins are useful for cloning vectors in mammalian cells. Generally, the origin of replication component is not needed for mammalian expression vectors (the SV40 origin may typically be used only because it contains the early promoter).

(3) Selective Marker Component

Expression and cloning vectors may contain a selective gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs methotrexate, neomycin, histidinol, puromycin, mycophenolic acid and hygromycin.

Another example of suitable selectable markers for mammalian cells are those that enable the identification of cells competent to take up the anti-Ep-CAM antibody nucleic acid, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity.

Alternatively, host cells (particularly wild-type hosts that contain endogenous DHFR) transformed or co-transformed with DNA sequences encoding Ep-CAM antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trpl gene present in the yeast plasmid YRp7 (Stinchcomb et al., Nature, 282: 39 (1979)). The trp1 gene provides a selection marker for a mutant strain of yeast lacking the ability to grow in tryptophan, for example, ATCC No. 44076 or PEP4-1. Jones, Genetics, 85: 12 (1977). The presence of the trpl lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene. Ura3-deficient yeast strains are complemented by plasmids bearing the ura3 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8: 135 (1990). Stable multi-copy expression vectors for secretion of mature recombinant human serum albumin by industrial strains of Kluyveromyces have also been disclosed. Fleer et al, Bio/Technology, 9: 968-975 (1991).

(4) Promoter Component

Expression and cloning vectors usually contain a promoter that is recognized by the host organism and is operably linked to the anti-Ep-CAM antibody nucleic acid. Promoters suitable for use with prokaryotic hosts include the arabinose (e.g., araB) promoter phoA promoter, β-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the anti-Ep-CAM antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Anti-Ep-CAM antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, most preferably cytomegalovirus, a retrovirus, hepatitis-B virus, Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297: 598-601 (1982) on expression of human α-interferon cDNA in mouse cells under the control of a thymidine kinase promoter from herpes simplex virus. Alternatively, the rous sarcoma virus long terminal repeat can be used as the promoter.

(5) Enhancer Element Component

Transcription of a DNA encoding the anti-Ep-CAM antibody of this invention by higher eukaryotes is often increased by inserting an enhancer sequence into the vector. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297: 17-18 (1982) on enhancing elements for activation of eukaryotic promoters. The enhancer may be spliced into the vector at a position 5′ or 3′ to the anti-Ep-CAM antibody-encoding sequence, but is preferably located at a site 5′ from the promoter.

(6) Transcription Termination Component

Expression vectors used in eukaryotic host cells (yeast, fingi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding Ep-CAM antibody. One useful transcription termination component is the bovine growth hormone polyadenylation region. See WO94/11026 and the expression vector disclosed therein. Another is the mouse immunoglobulin light chain transcription terminator.

(7) Selection and Transformation of Host Cells

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for Ep-CAM antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastors (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated anti-Ep-CAM antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become routine procedure. Examples of useful mammalian host cell lines are Chinese hamster ovary cells, including CHOK1 cells (ATCC CCL61), DXB-11, DG-44, and Chinese hamster ovary cells/-DHFR(CHO, Urlaub et al., Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture, [Graham et al., J. Gen Virol. 36: 59 (1977)]; baby hamster kidney cells (BHK, ATCC CCL 10); mouse sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383: 44-68 (1982)); MRC 5 cells; FS4 cells; and a human hepatoma line (Hep G2).

Host cells are transformed or transfected with the above-described expression or cloning vectors for anti-Ep-CAM antibody production and cultured in conventional nutrient media modified as appropriate for inducing promoters, selecting transformants, or amplifying the genes encoding the desired sequences. In addition, as described in detail in Example 1-5 below, novel vectors and transfected cell lines with multiple copies of transcription units separated by a selective marker are particularly useful and preferred for the expression of human engineered antibodies that target Ep-CAM.

(8) Culturing the Host Cells

The host cells used to produce the anti-Ep-CAM antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. No. 4,767,704; 4,657,866; 4,927,762; 4,560,655; or 5,122,469; WO90103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

(ix) Purification of Anti-Ep-CAM Antibody

When using recombinant techniques, the antibody can be produced intracellularly, in the periplasmic space, or directly secreted into the medium, including from microbial cultures. If the antibody is produced intracellularly, as a first step, the particulate debris, either host cells or lysed fragments, is removed, for example, by centrifugation or ultrafiltration. Better et al. Science 240: 1041-1043 (1988); ICSU Short Reports 10: 105 (1990); and Proc. Natl. Acad. Sci. USA 90: 457-461 (1993) describe a procedure for isolating antibodies which are secreted to the periplasmic space of E. coli. (See also, [Carter et al., Bio/Technology 10: 163-167 (1992)].

The antibody composition prepared from microbial or mammalian cells can be purified using, for example, hydroxylapatite chromatography cation or avian exchange chromatography, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62: 1-13 (1983)). Protein G is recommended for all mouse isotypes and for human γ3 (Guss et al., EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH 3 domain, the Bakerbond ABX™resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

Preferred methods for the purification of human engineered anti-EP-CAM antibodies of the invention are described in Example 8 below.

C. Pharmaceutical Formulations

Therapeutic formulations of the antibody are prepared for storage by mixing the antibody having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. For example, it may be desirable to further provide an immunosuppressive agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

D. Non-Therapeutic Uses for the Antibody

The antibodies of the invention may be used as affinity purification agents. In this process, the antibodies are immobilized on a solid phase such a Sephadex resin or filter paper, using methods well known in the art. The immobilized antibody is contacted with a sample containing the Ep-CAM protein (or fragment thereof) to be purified, and thereafter the support is washed with a suitable solvent that will remove substantially all the material in the sample except the Ep-CAM protein, which is bound to the immobilized antibody. Finally, the support is washed with another suitable solvent, such as glycine buffer, pH 5.0, that will release the Ep-CAM protein from the antibody.

Anti-Ep-CAM antibodies may also be useful in diagnostic assays for Ep-CAM protein, e.g., detecting its expression in specific cells, tissues, or serum.

For diagnostic applications, the antibody typically will be labeled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991) for example and radioactivity can be measured using scintillation counting.

(b) Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra, for example. Fluorescence can be quantified using a fluorimeter.

(c) Various enzyme-substrate labels are available and U.S. Pat. No. 4,275,149 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocydic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed J. Langone & H. Van Vunakis), Academic press, N.Y., 73: 147-166 (1981).

Examples of enzyme-substrate combinations include, for example:
(i) Horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB));
(ii) alkaline phosphatase (AP) with para-Nitrophenyl phosphate as chromogenic substrate; and
(iii) β-D-galactosidase (β-D-Gal) with a chromogenic substrate (e.g., p-nitrophenyl-β-D-galactosidase) or fluorogenic substrate 4-methylumbelliferyl-β-D-galactosidase.

Numerous other enzyme-substrate combinations are available to those skilled in the art. For a general review of these, see, e.g., U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In another embodiment of the invention, the anti-Ep-CAM antibody need not be labeled, and the presence thereof can be detected using a labeled antibody which binds to the Ep-CAM antibody.

The antibodies of the present invention may be employed in any known assay method, such as competitive binding assays, direct and indirect sandwich assays, and immunoprecipitation assays. Zola, Monoclonal Antibodies: A Manual of Techniques, pp.147-158 (CRC Press, Inc. 1987).

Competitive binding assays rely on the ability of a labeled standard to compete with the test sample analyte for binding with a limited amount of antibody. The amount of Ep-CAM protein in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the tumor sample may be fresh or frozen or may be embedded in paraffin and fixed with a preservative such as formalin, for example.

The antibodies may also be used for in vivo diagnostic assays. Generally, the antibody is labeled with a radionuclide (such as ¹¹¹In, ⁹⁹Tc, ¹⁴C, ¹³¹I, ¹²⁵I, ³H, ³²P or ³⁵S) so that the tumor can be localized using immunoscintiography.

E. Diagnostic Kits

As a matter of convenience, the antibody of the present invention can be provided in a kit, i.e., a packaged combination of reagents in predetermined amounts with instructions for performing the diagnostic assay. Where the antibody is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

F. Therapeutic Uses for the Antibody

It is contemplated that an anti-Ep-CAM antibody of the present invention may be used to treat the various Ep-CAM mediated diseases, conditions and disorders, particularly to treat Ep-CAM expressing cancer cells, including, for example, an epithelial cancer cell such as an epithelial carcinoma, and most particularly to treat tumor cell metastases. It is contemplated that an anti-Ep-CAM antibody of the present invention may be used to bind to, contact, inhibit the growth of, inhibit the metastasis of and/or kill an Ep-CAM expressing cell, including an Ep-CAM expressing cancer cell, alone or in combination with another agent such as a chemotherapeutic agent. An anti-Ep-CAM antibody of the present invention may be administered to a subject with an Ep-CAM mediated disease, condition or disorder, including a subject with a cancer such as an adenocarcinoma (e.g., of the breast, lung, prostate, gastrointestinal tract (e.g., colon/large intestine, small intestine, rectum, pancreas, stomach, esophagus) ovary, cervix, vagina, kidney, liver, bladder, bile duct, gallbladder, thyroid, endometrium, other organ or tissue), including a subject with an advanced adenocarcinoma.

The anti-Ep-CAM antibody is administered by any suitable means, including parenteral, subcutaneous, intraperitoneal, intrapulmonary, and intranasal, and, if desired for local immunosuppressive treatment, intralesional administration (including perfusing or otherwise contacting the graft with the antibody before transplantation). Parenteral infusions include intramuscular, intravenous, intraarterial, intraperitoneal, or subcutaneous administration. In addition, the anti-Ep-CAM antibody is suitably administered by pulse infusion, particularly with declining doses of the antibody. Preferably the dosing is given by injections, most preferably intravenous or subcutaneous injections, depending in part on whether the administration is brief or chronic.

For the prevention or treatment of disease, the appropriate dosage of antibody will depend on the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 10 μg/kg to 5 mg/kg or about 30 μg/kg to 1 mg/kg of antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

The antibody composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The therapeutically effective amount of the antibody to be administered will be governed by such considerations, and is the minimum amount necessary to prevent, ameliorate, or treat the Ep-CAM mediated disease, condition or disorder, including treating various Ep-CAM mediated diseases, conditions and disorders, particularly to treat Ep-CAM expressing cancer cells, and most particularly to treat tumor cell metastases. Such amount is preferably below the amount that is toxic to the host or renders the host significantly more susceptible to infections.

The antibody need not be, but is optionally formulated with one or more agents currently used to prevent or treat the disorder in question. For example, in cancer, the antibody may be given in conjunction with chemo therapeutic agent or in ADEPT as described above. The effective amount of such other agents depends on the amount of anti-Ep-CAM antibody present in the formulation, the type of disease, condition or disorder or treatment, and other factors discussed above. These are generally used in the same dosages and with administration routes as used hereinbefore or about from 1 to 99% of the heretofore employed dosages.

G. Articles of Manufacture

In another embodiment of the invention, an article of manufacture containing materials useful for the treatment of the diseases, disorders or conditions described above is provided, including for treatment of cancer. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the anti-Ep-CAM antibody. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

Other aspects, versions, and advantages of the present invention will be understood upon consideration of the following illustrative examples, wherein Example 1 addresses the construction of expression vectors according to the present invention that contain multiple copies of a given transcription unit; Example 2 addresses development of a mouse-human chimeric ING-1 producing CHO-K1 cell line, Clone 40, by transfection with a two-transcription unit vector, pING1932 and the development of Clone 146, by transfection with a two-transcription unit vector, pING1937; Example 3 addresses the development of Clones 259 and 373 by sequential transfection of Clone 146 with two-transcription unit vector pING1957 and Clone 373 by sequential transfection of subclone 146.3 with two-transcription unit vector pING1959; Example 4 addresses the development Clone 132 by sequential transfection of Clone 373 with two-transcription unit vector pING1957; Example 5 addresses the development of Clones 53 and 157 by transfection with the two-transcription unit vector pING1959, containing one copy of each of the human engineered ING-1 light and heavy chain genes, and the development of Clone 17 transfected with the four gene vector pING1964, containing two copies of each of the human engineered ING-1 light and heavy chain genes; Example 6 addressing the binding activity of exemplary immunoglobulin polypeptides; Example 7 describing the development of a direct binding ELISA assay with soluble EpCam; Example 8 describes the purification of immunoglobulin polypeptides from cultured cell lines; Example 9 describes in vitro activity of human engineered anti-Ep-CAM antibodies in ADCC (antibody-dependent cellular eytotoxicity) and CDC (complement dependent eytotoxicity) studies; and Example 10 describes pharmacokinetic studies and in vivo activity studies in a variety of tumor models in animals.

### EXAMPLE 1

### Preparation of Human Engineered Variable Regions and Construction of Vectors for Expression of Human Engineered Antibodies to EP-CAM

This example describes the preparation of human engineered immunoglobulin sequences, including human engineered variable regions, and the construction of vectors useful for expression of human engineered antibodies, including vectors comprising multiple copies of exemplary transcription units encoding human engineered variable regions. These exemplary vector constructs comprise gene sequences encoding immunoglobulin polypeptides of interest, including human engineered immunoglobulin gene sequences that are light and/or heavy chain sequences that target Ep-CAM. A mouse-human chimeric antibody (ING-1) has been described in U.S. Pat. Nos. 5,576,184, 5,843,685 and 6,461,824 (all incorporated by reference herein) and has been deposited as ATCC HB 9812. This chimeric antibody has murine variable regions and human gamma 1 and kappa constant regions. Mouse-human chimeric heavy and light chain vectors as described in these patents are useful in the construction of novel vectors as described in this example.

A. Construction of Vectors Comprising Mouse-Human Chimeric ING-1 Light Chain Gene

Vectors comprising sequences encoding mouse-human chimeric ING-1 light (SEQ ID NOS: 1 and 2) and heavy chains (SEQ ID NOS: 3 and 4) which incorporate the necessary elements for optimal expression in CHO-K1 cells have been constructed. These ING-1 vectors serve both as the starting point for construction of human engineered antibody genes and have been used to develop CHO-K1 cell lines expressing mouse-human chimeric ING-1. The expression vectors described below have a CMV promoter and a mouse kappa light chain 3′ un-translated region and transcription units encoding selective gene markers, and light and/or heavy chain sequences.

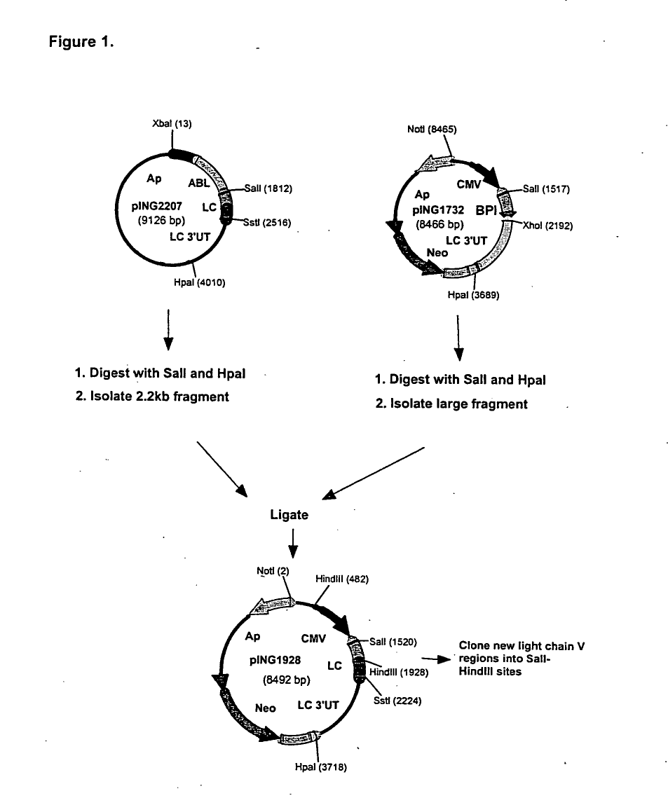

A mouse-human chimeric ING-1 light chain vector, pING1928, was constructed by digesting pING2207 (see, e.g., U.S. Pat. No. 5,576,184), comprising a mouse-human chimeric ING-1 light chain gene (SEQ ID NOS: 1) fused to a mouse light chain 3′ untranslated region, with SalI plus HpaI and isolating the ˜2200 bp fragment comprising a light chain gene (FIG. 1). This fragment was ligated to a ˜6300 bp SalI-HpaI vector fragment from pING1732, placing the mouse-human chimeric ING-1 light chain gene under control of the CMV promoter and mouse light chain 3′ untranslated region (FIG. 1). Alternative light chain variable region gene sequences may be cloned into the SalI HindIII sites of pING1928, including human engineered antibody variable region gene sequences as described below.

B. Construction of Vectors Comprising Mouse-Human Chimeric ING-1 Heavy Chain Gene

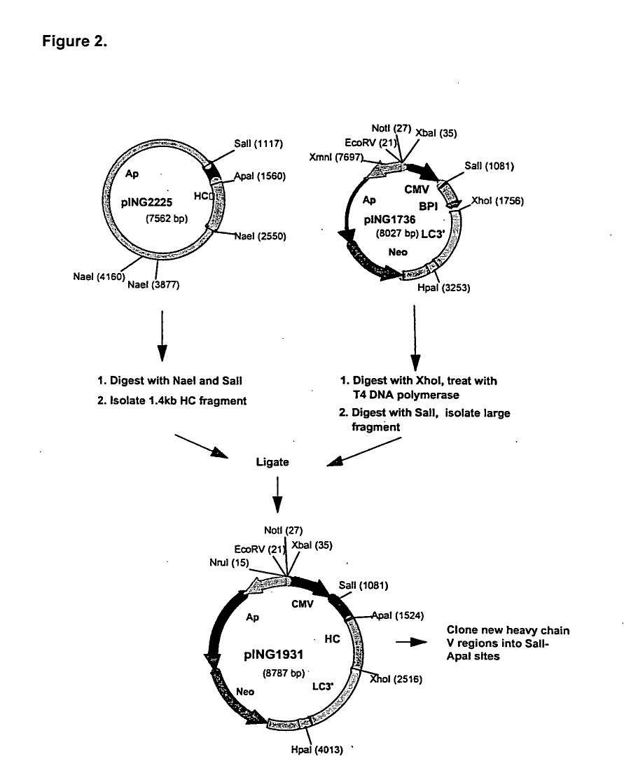

A mouse-human chimeric ING-1 heavy chain vector, pING1931, was constructed by digesting pING2225 (See, e.g., U.S. Pat. No. 5,576,184), comprising a mouse-human chimeric ING-1 heavy chain gene (SEQ ID NO: 3) with SalI plus NaeI and isolating the ˜1433 bp fragment comprising the heavy chain gene sequence (FIG. 2). This fragment was ligated to the 7352 bp vector fragment from pING1736 (described in Example 1 above, similar to pING1740 except that it contains the neo instead of the gpt gene) which had been digested with XhoI, treated with T4 DNA polymerase in the presence of deoxyribonucleotides to blunt end, and then with SalI placing the mouse-human chimeric ING-1 heavy chain gene (SEQ ID NO: 3) under control of the human CMV promoter and the mouse light chain 3′ untranslated region (FIG. 2). Alternative heavy chain variable region gene sequences may be cloned into the Sal-ApaI sites of pING1931, including human engineered antibody variable region gene sequences as described below.

C. Construction of Mouse-Human Chimeric Light Plus Heavy Chain Vectors Mouse-Human Chimeric (Two Gene Vectors)

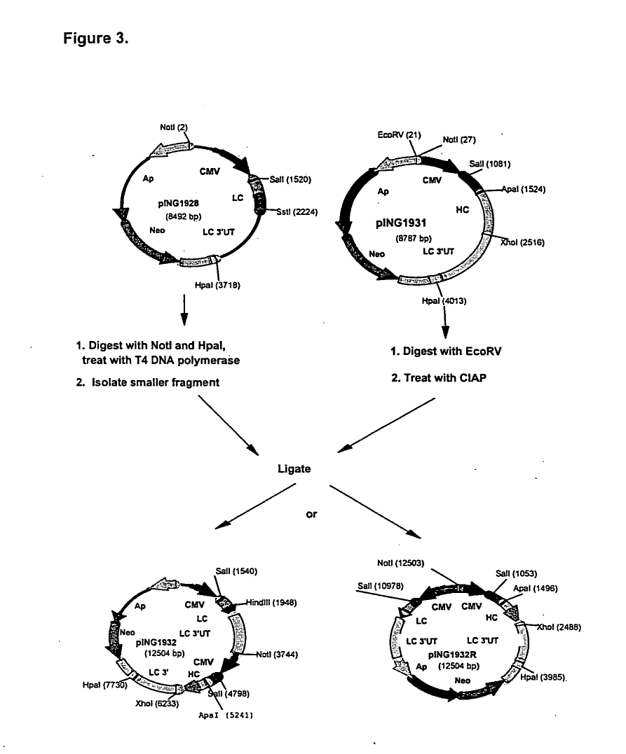

Vectors comprising mouse-human chimeric ING-1 light plus heavy chain gene sequences (SEQ ID NOS: 1 and 3) were constructed using pING1928 and pING1931 (FIG. 3). pING1931 was digested with EcoRV and treated with calf intestinal alkaline phosphatase (CIAP). EcoRV cuts at a unique site adjacent (and counterclockwise on a circular map) to a unique NotI site. pING1928 was digested with NotI and HpaI, and then treated with T4 DNA polymerase in the presence of deoxyribonucleotides to blunt end. The 3720 bp fragment comprising a mouse-human chimeric light chain gene (SEQ ID NO: 1) was purified and ligated with EcoRV-digested pING1931 comprising a mouse-human chimeric heavy chain gene (SEQ ID NO: 3). Both possible orientations, represented by pING1932 and pING1932R, were obtained as shown in FIG. 3.

D. Preparation of Human Engineered Variable Regions

Human engineering of antibody variable domains has been described by Studnicka [See, e.g., Studnicka et al. U.S. Pat. No. 5,766,886; Studnicka et al. Protein Engineering 7: 805-814 (1994)] as a method for reducing immunogenicity while maintaining binding activity of antibody molecules. According to the method, each variable region amino acid has been assigned a risk of substitution. Amino acid substitutions are distinguished by one of three risk categories: (1) low risk changes are those that have the greatest potential for reducing immunogenicity with the least chance of disrupting antigen binding; (2) moderate risk changes are those that would further reduce immunogenicity, but have a greater chance of affecting antigen binding or protein folding; (3) high risk residues are those that are important for binding or for maintaining antibody structure and carry the highest risk that antigen binding or protein folding will be affected.

Variable regions of the light and heavy chains of the mouse-human chimeric ING-1 were human engineered using this method. To apply this method to the mouse variable regions of the mouse-human chimeric ING-1 antibody, amino acid residues that are candidates for modification according to the method at low risk positions were identified by aligning the amino acid sequences of the murine variable regions with a human variable region sequence. Any human variable region can be used, including an individual V_{H} or V_{L} sequence or a human consensus V_{H} or V_{L} sequence. The amino acid residues at any number of the low risk positions or at all of the low risk positions, can be changed. As described below, for the murine ING-1 variable regions, for each position where the murine and human amino acid residues differed at low risk positions, an amino acid modification was introduced in order to generate novel sequences with low risk modifications.

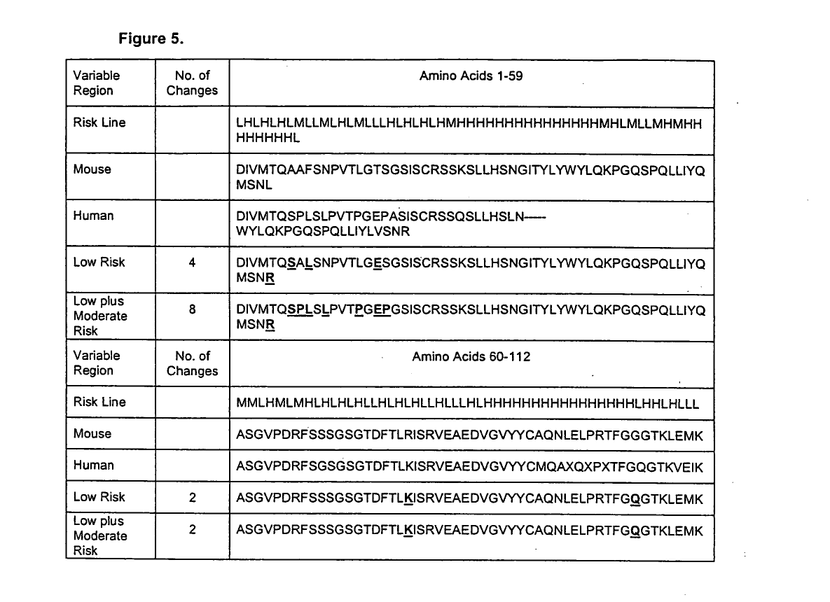

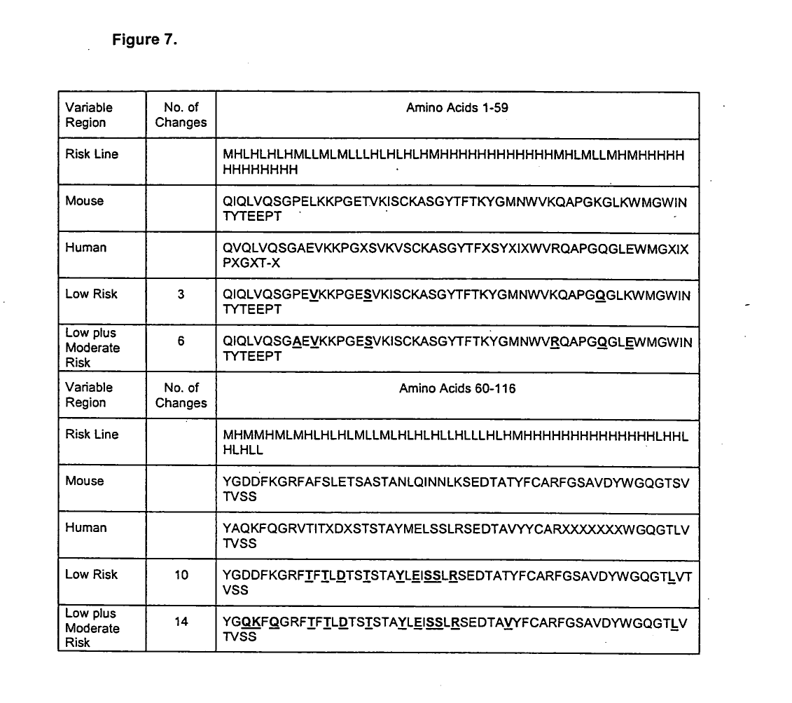

Specifically, the heavy and light chain amino acid variable region sequences were aligned with a human consensus amino acid variable region sequence for Gamma heavy chain subgroups 1 through 3 (shown in FIG. 7 as the human sequence) and a human consensus amino acid variable region sequence for Kappa light chain subgroups 1 through 4 (shown in FIG. 5 as the human sequence), respectively. Six amino acid modifications were made to the light chain and 13 modifications were made to the heavy chain. The positions and nature of these changes in the light and heavy chain variable regions are underlined in FIG. 5 (for the light chain variable region) and FIG. 7 (for the heavy chain variable region). Also shown in each of FIGS. 5 and 7 is the risk line which identifies low, moderate and high risk positions according to this method. It also should be noted that low risk positions where no changes were made represent variable region amino acids that are conserved between the original murine (Br-1) antibody [see, e.g., U.S. Pat. No. 5,576,184 and Robinson et al., Hum. Antibodies Hybridomas 2:84-93 (1991), incorporated by reference herein in their entirety] and the human consensus sequence. Once each of the human engineered variable region sequences was designed to include the selected low risk amino acid modifications, DNA fragments encoding low risk light and heavy chain variable regions were constructed using 6 overlapping synthetic oligionucleotides for each chain as described below.

E. Construction of Vectors Comprising Human Engineered ING-1 Light Chain Gene

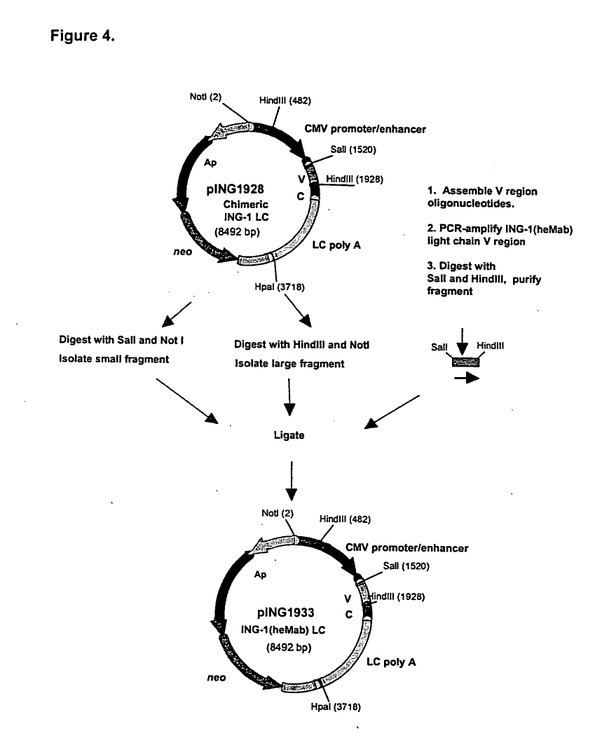

A human engineered ING-1 light chain vector, pING1933 (FIG. 4), was constructed by digesting pING1928 (FIG. 1), containing a mouse-human chimeric ING-1 light chain gene, with SalI plus NotI and isolating the ˜1518 bp fragment with a CMV promoter and separately digesting pING1928 with HindIII plus NotI and isolating the 6566 bp fragment comprising a human light chain constant region, a mouse light chain 3′ untranslated region and a neo gene for selection of G418-resistant transfectants. These fragments were ligated to a 400 bp PCR-generated SalI-HindIII fragment comprising an ING-1 light chain variable region human engineered with a total of 6 low risk amino acid substitutions (FIG. 5; SEQ ID NO: 5), placing the low risk human engineered ING-1 light chain gene under control of a CMV promoter and mouse light chain 3′ untranslated region. Low risk changes as well as low plus moderate risk changes in an ING-1 light chain variable region are shown in FIG. 5. For the light chain, a total of 6 low risk changes were made for a low risk variable region (SEQ ID NO: 6) as described, and separately a total of 10 low plus moderate risk changes were made for a low plus moderate risk variable region (SEQ ID NOS: 7 AND 8). The vector pING1933 comprises a PCR-generated human engineered ING-1 light chain variable region with 6 low risk changes incorporated. A DNA fragment encoding a low risk modified light chain variable region was constructed using 6 overlapping oligonucleotides KL1 (SEQ ID NO: 9), KL2 (SEQ ID NO: 10), KL3 (SEQ ID NO: 11), KL4 (SEQ ID NO: 12), KL5 (SEQ ID NO: 13), and KL6 (SEQ ID NO: 14). These segments were annealed to each other, extended with DNA polymerase and then the assembled variable region amplified by PCR using 5′ forward primer KF (SEQ ID NO: 15) and 3′ reverse primer KR (SEQ ID NO: 16), digested with SalI and Hind III to yield a restriction fragment that was cloned directly into expression vector pING1928 to generate pING1933 as shown in FIG. 4. Another expression vector, pING1939, was constructed using a similar method and is like pING1933 except that pING1939 comprises an ING-1 light chain variable region human engineered with the low plus moderate risk changes as shown in FIG. 5 (SEQ ID NOS: 7 AND 8). The low plus moderate risk modified light chain variable region was constructed using 6 overlapping oligonucleotides, including 5 used in the construction of the low risk modified variable region described above KL1 (SEQ ID NO: 9), KM2 (SEQ ID NO: 17), KL3 (SEQ ID NO: 11), KL4 (SEQ ID NO: 12), KL5 (SEQ ID NO: 13) and KL6 (SEQ ID NO: 14), as well as a new oligonucleotide KM2 (SEQ ID NO: 17).

F. Construction of Vectors Comprising Human Engineered ING-1 Heavy Chain Gene

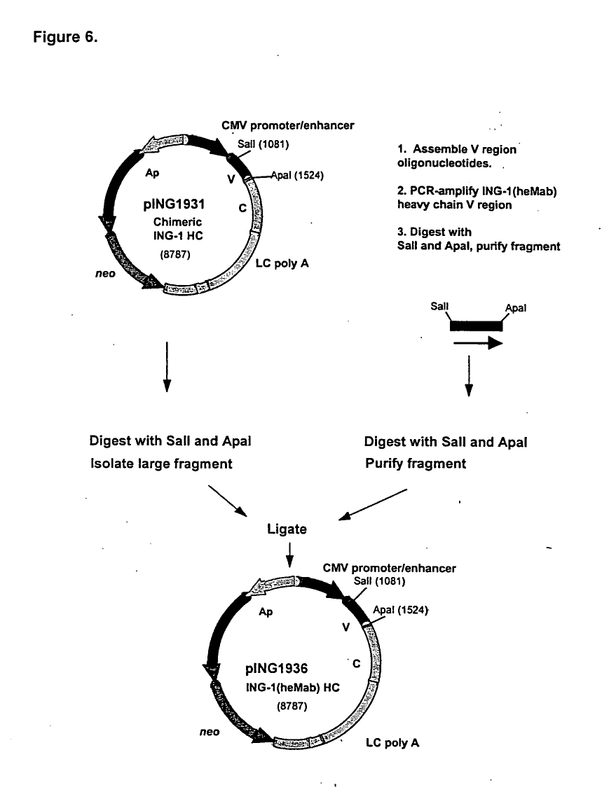

A human engineered ING-1 heavy chain vector, pING1936 (FIG. 6), was constructed by digesting pING1931, containing mouse-human chimeric ING-1 heavy chain with SalI plus ApaI and isolating the 8344 bp fragment comprising a CMV promoter, heavy chain constant region, light chain 3′ untranslated region and a neo gene for selection of G418-resistant transfectants. This fragment was ligated to the ˜450 bp PCR-generated SalI-ApaI fragment comprising an ING-1 heavy chain variable region human engineered with a total of 13 low risk amino acid substitutions (FIG. 7; SEQ ID NO: 18), placing the low risk human engineered ING-1 heavy chain gene under control of a CMV promoter and mouse light chain 3′ untranslated region. Low risk changes as well as low plus moderate risk changes in an ING-1 heavy chain variable region are shown in FIG. 7. For the heavy chain, a total of 13 low risk changes were made for a low risk variable region (SEQ ID NO: 19) as described, and separately a total of 20 low plus moderate risk changes were made for a low plus moderate risk variable region (SEQ ID NO: 20 and 21). The vector pING1936 separately attached contains a PCR-generated human engineered ING-1 heavy chain variable region with 13 low risk changes incorporated. A DNA fragment encoding the heavy chain variable region was constructed using 6 overlapping oligonucleotides GL1 (SEQ ID NO: 22), GL2 (SEQ ID NO: 23), GL3 (SEQ ID NO: 24), GL4 (SEQ ID NO: 25), GL5 (SEQ ID NO: 26), and GL6 (SEQ ID NO: 27). These segments were annealed to each other, extended with DNA polymerase and then the assembled variable region amplified by PCR using a 5′ forward primer GF (SEQ ID NO: 28) and a 3′ reverse primer GR (SEQ ID NO: 29), digested with SalI and ApaI to yield a restriction fragment that was cloned directly into expression vector pING1931 to generate pING1936 as shown in FIG. 6. Another expression vector, pING1942, was contructed using a similar method and is like pING1936 except that pING1492 comprises an ING-1 heavy chain variable region human engineered with the low plus moderate risk changes as shown in FIG. 7 (SEQ ID NOS: 20 and 21). The low plus moderate risk modified heavy chain variable region was constructed using 6 overlapping oligonucleotides, including 2 used in the construction of the low risk modified variable region described above: GL1 (SEQ ID NO: 22), GM2 (SEQ ID NO: 30), GM3 (SEQ ID NO: 31), GM4 (SEQ ID NO: 32), GM5 (SEQ ID NO: 33), and GL6 (SEQ ID NO: 27).

G. Construction of Vectors Comprising Human Engineered ING-1 Light Plus Heavy Chain Genes (Two Gene Vectors)

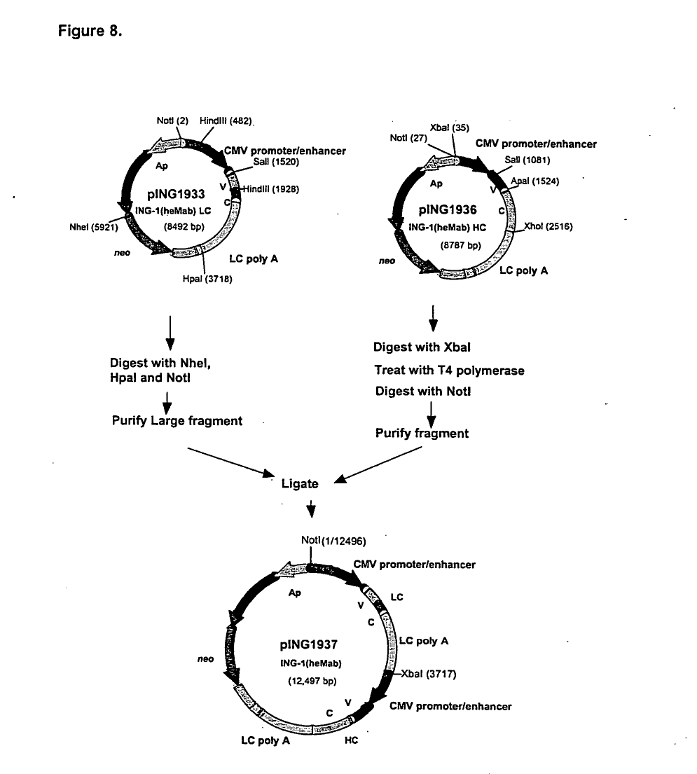

A vector pING1937, comprising human engineered ING-1 light plus heavy chain genes, was constructed using pING1933 and pING1936 (FIG. 8). pING1936 was digested with XbaI, treated with T4 polymerase and then digested with NotI. The 8780 bp restriction fragment was purified. pING1933 was digested with NheI, then NotI and HpaI, and the ˜3716 bp fragment comprising a human engineered light chain gene was purified and ligated with the XbaI-NotI-digested pING1936 to generate pING1937, which has a neo gene for selection of G418-resistant transfectants. The variable region DNAs were re-sequenced before being used to construct light plus heavy chain expression vectors. The features of pING1937 are summarized in Table 1.

TABLE 1
Description of pING1937 vector.

| Plasmid region | Start nt | End nt | Description |
|---|---|---|---|
| NotI-HindIII | 1 | 479 | =pUC12 2616-399 (includes pBR322 4291-4361, |
| | | | 2069-2354 and part of lac gene) |
| HindIII- | 479 | 643 | upstream region of Abelson murine leukemia virus |
| ½BamHI | | | 3′LTR enhancer/promoter (=4627-4804 of Reddy et |
| | | | al., 1983 sequence; ref 8) |
| ½HincII-BamHI | 643 | 1414 | hCMV promoter (=−598 to 174 of Boshart et al., ref |
| | | | 1; includes splice donor) |
| BamHI-SalI | 1414 | 1517 | SV40 16S splice acceptor (1654-1741 = SV40 1410-1497) |
| SalI-HindIII | 1517 | 1925 | ING-1(heMab) light chain V region |
| HindIII-NS^{a} | 1925 | 2263 | ING-1 light chain(heMab) C region (kappa) |
| NS-BamHI | 2263 | 3582 | LC genomic DNA including poly A site |
| BamHI- | 3582 | 3889 | upstream region of Abelson murine leukemia virus |
| ½BamHI | | | 3′LTR enhancer/promoter |
| ½HincII-BamHI | 3889 | 4660 | hCMV promoter, including splice donor |
| BamHI-SalI | 4660 | 4763 | SV40 16S splice acceptor |
| SalI-ApaI | 4763 | 5206 | ING-1 (heMab) heavy chain V region |
| ApaI-XhoI | 5206 | 6198 | ING-1 heavy chain(heMab) C region (gamma-1) |
| XhoI-BamHI | 6198 | 7562 | LC genomic DNA including poly A site |
| BamHI-½BclI | 7562 | 7802 | SV40 polyadenylation (=SV40 2532-2774) |
| ½BstYI-NS^{a} | 7802 | 8409 | SV40 small T intron (=SV40 4769-4099) |
| ½SalI-NheI | 8409 | 9898 | bacterial neomycin phoshotransferase (neo) gene |
| | | | from pSV2neo (coding region = 9545-8753) |
| NheI-PvuII | 9898 | 10242 | SV40 promoter (=SV40 5172-272) |
| PvuII-NotI | 10242 | 12496 | bacterial origin of replication and beta-lactamase |
| | | | (ampicillin resistance) gene (=pBR322 2069-4290) |

^{a}NS - restriction site not identified.

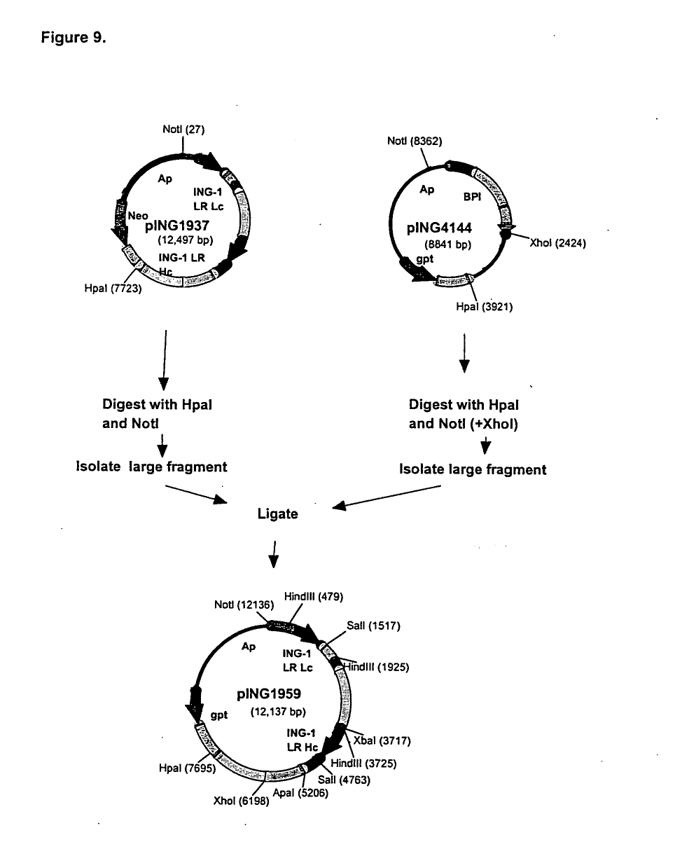

The vector, pING1959, which is similar to pING1937 except that it has a gpt gene for selection of mycophenolic acid-resistant transfectants, was constructed by ligating the ˜7696 bp HpaI-NotI fragment from pING1937 (comprising human engineered ING-1 light and heavy chain genes each fused to a CMV promoter and light chain 3′ untranslated region) with a ˜4441 bp HpaI-NotI fragment from pING4144 (described in Example 1 above) comprising a gene encoding gpt as shown in FIG. 9.

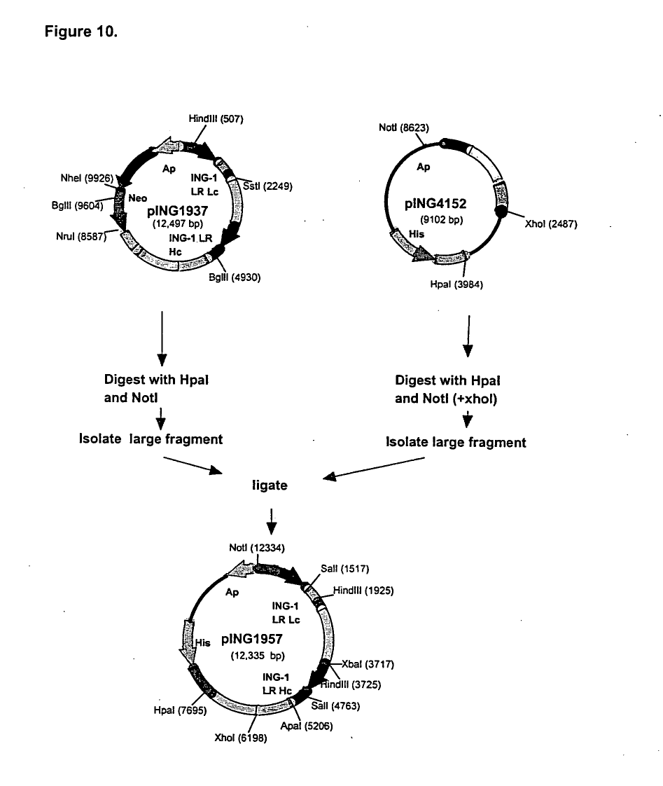

The vector, pING1957, which is similar to pING1937 and pING1959 except that it has a his gene for selection of histidinol-resistant transfectants, was constructed by ligating the ˜7696 bp HpaI-NotI fragment from pING1937 (as described above) with a ˜4639 bp HpaI-NotI fragment from pING4152 comprising a his gene as shown in FIG. 10.

Another vector pING1944 was constructed by similar methods used in the construction of pING1937 described above, and is similar to pING1937 except that pING1944 was constructed using pING1939 in place of pING1933 and using pING1942 in place of pING1936. The resulting vector, pING1944 comprises light chain and heavy chain variable region sequences (SEQ ID NOS: 7 and 8) with both the low plus moderate risk substitutions as shown in FIGS. 5 and 7. Thus, expression vectors for both low risk ING-1 (pING1937) and low plus moderate risk ING-1 (pING1944) were prepared.

H. Construction of Vectors Comprising Two Copies of Human Engineered ING-1 Light and Heavy Chain Genes (Four Gene Vectors)

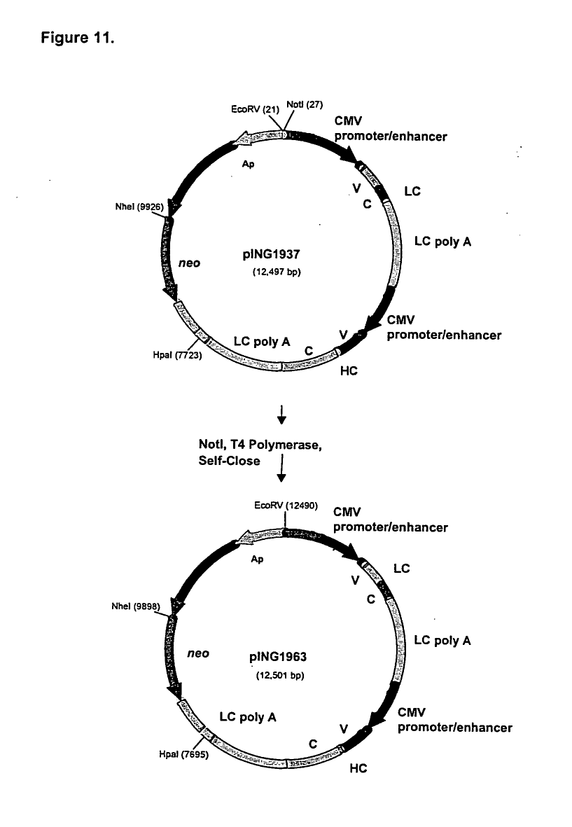

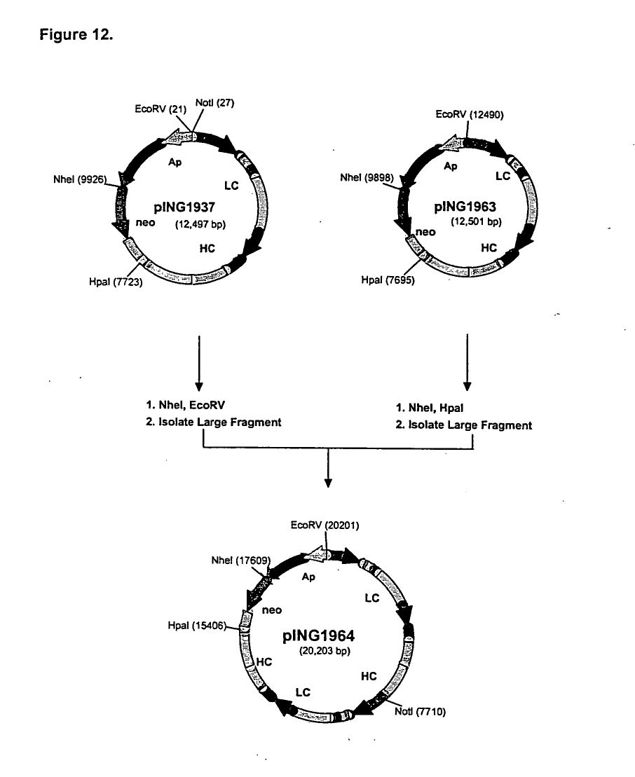

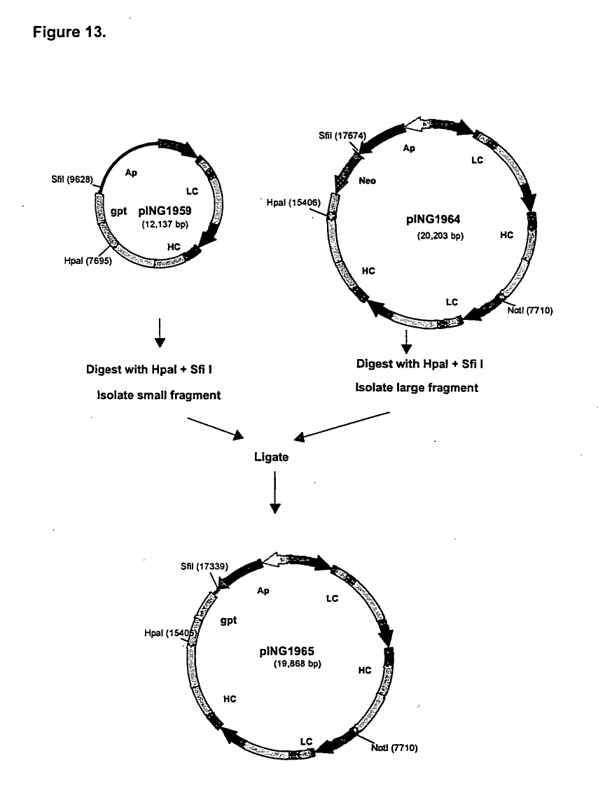

A human engineered ING-1 heavy plus light chain vector, pING1937, was treated with NotI, T4 DNA polymerase in the presence of deoxyribonucleotides to blunt end and allowed to self-close, destroying the NotI site and generating the vector pING1963 lacking a NotI site as shown in FIG. 11. The vector pING1937 was then digested with NheI and EcoRV and the ˜9905 bp fragment was purified and ligated with the ˜10,298 bp NheI-HpaI fragment from pING1963 to generate the vector pING1964 as shown in FIG. 12 which comprises four ING-1 genes (a four gene vector). pING1964 has two copies of human engineered ING-1 light chain genes and two copies of ING-1 heavy chain genes, with each of the four genes under control of a CMV promoter and light chain 3′ untranslated region and a neo gene for selection of G418-resistant transfectants. A vector, pING1965, which is similar to pING1964 except that it contains a gpt gene for selection of mycophenolic acid-resistant transfectants was constructed by ligating the 1933 bp HpaI-SfiI fragment from pING1959 with the ˜17,935 bp HpaI-SfiI fragment from pING1964 as shown in FIG. 13.

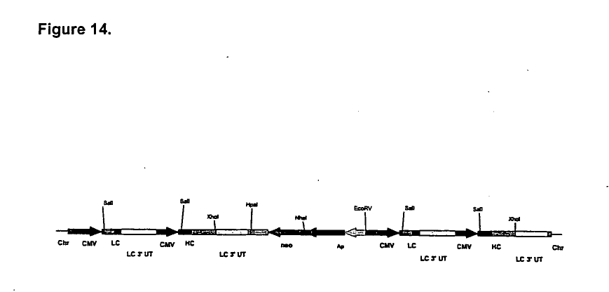

Digestion of pING1964 or pING1965 at the unique NotI site yields a linear restriction fragment containing four transcription units: two copies of human engineered ING-1 light plus heavy chain genes configured so that a selective marker gene, neo, or gpt, respectively, is positioned between the two identical light and heavy chain transcription units. Viewed as linear NotI-digested DNA, the order of elements within the vector(s) is as follows: CMV promoter, light chain gene, light chain 3′ untranslated region, CMV promoter, heavy chain gene, light chain 3′ untranslated region, neo (pING1964) or gpt (pING1965) genes, bla (Ampr) gene, CMV promoter, light chain gene, light chain 3′ untranslated region, CMV promoter, heavy chain gene, light chain 3′ untranslated region, (FIG. 14).

### EXAMPLE 2

### Development and Characterization of Transfected Clones and Cell Lines for Expression of Human Engineered Antibodies

This example describes the development and characterization of clones and cell lines transfected with exemplary vectors, for example, as described in Example 1. The development and characterization of immunoglobulin producing cell lines is described from transfections, for example, human engineered anti-Ep-CAM, with two gene vectors as described in Example 1.

A. pING1932 and pING1932R

The expression vectors, pING1932 and pING1932R described in Example 1 were transfected into Ex-Cell 301-adapted CHO-K1 cells. CHO-K1 cells adapted to suspension growth in Ex-Cell 301 medium were typically electroporated with 40 μg of linearized vector. Both pING1932 and pING1932R contain a unique NotI site. In preparation of DNA for transfection, digestion at NotI results in linear DNA such that light and heavy chain genes, under the control of a CMV promoter and light chain 3′ untranslated region, are separated by the selective marker gene when inserted into the CHO chromosome. With pING1932, the heavy and light chains are oriented in the same direction, whereas in pING1932R, they are oriented in opposite directions.

The cells were plated in 96 well plates containing Ex-Cell 301 medium supplemented with 2% FBS and G418. A total of 155 and 168 clones were screened in 96 well plates for pING1932 and pING1932R, respectively. The top 22 clones for each transfection were transferred to 24 well plates containing Ex-Cell 301 medium without FBS.

A productivity test was performed in 24 well plates in Ex-Cell 301 medium with or without 2% FBS. Cells were grown to extinction and culture supernatants tested for levels of secreted antibody by an immunoglobulin ELISA assay for IgG. The results demonstrated that the pING1932 transfectants generally secreted higher levels of immunoglobulin polypeptide than the pING1932R transfectants. Interestingly, in some cases, the levels of secreted immunoglobulin polypeptides were higher in the medium without FBS than in those supplemented with FBS. The top transfectants from each group secreted in the range from about 7 μg/ml IgG to more than about 30 μg/ml IgG.

The top 7 clones from the pING1932 transfection (including, for example, Clones 27, 40 and 82) and the top clone from the pING1932R transfection (Clone 168R) were transferred to shake flasks containing Ex-Cell 301 medium. As soon as the cells were adapted to suspension growth, a shake flask test was performed with these clones in Ex-Cell 301 medium with and without 2% FBS. The cells were grown for up to 10 days in 125 ml Erlenmeyer flasks containing 25 ml media. The flasks were sealed for the most of the incubation period and the levels of immunoglobulin polypeptide in the culture medium were determined by IgG ELISA at the end of the incubation period. The results of the initial shake flask test demonstrated that the top clone (Clone 40) secreted up to ˜66 μg/ml. In many cases, there was little difference in productivity between cultures grown with and without FBS.

The initial shake flask test was performed with flasks that were not opened regularly during the incubation period. Because introducing a gas exchange step at least every other day was previously found to significantly influence the productivity of certain polypeptide-producing CHO-K1 clones, this approach was evaluated with Clones 27, 40, 82 and 168R. Cells were seeded at 1.5×10⁵ cells/ml into duplicate 125 ml Ehrlenmeyer flasks in 25 ml Ex-Cell 301 medium supplemented with 1% FBS and incubated at 37° C., 100 RPM. One set of flasks remained sealed for the duration of the incubation, while the other set was opened every day for cell counts and aeration. The results demonstrated that cells grown in flasks that were periodically opened expressed immunoglobulin polypeptide at a higher level (for example, from about 50 μg/ml to about 116 μg/ml) than those in which the flasks remained closed (for example, from about 35 μg/ml to about 81 μg/ml). These results also corresponded to those obtained in the first shake flask test (for example, from about 45 μg/ml to about 66 μg/ml), although the conditions were slightly different (1% FBS in the second test vs. 2% FBS in the first test).

The cultures that were opened periodically were also examined for growth and productivity at various times. The results of this analysis for Clones 27 and 40 indicated that the cells produced mouse-human chimeric antibody during both the log and the stationary phases.

B. pING1937

The expression vector pING1937, one copy each of the human engineered ING-1 low risk light and heavy chain genes and the neo (G418-resistant) gene, was linearized by digested with XbaI followed by transfection into serum-free adapted CHO-K1 cells in Ex-Cell 301 medium. G418-resistant transfectants were selected and screened for immunoglobulin polypeptide expression. Clone 146 was selected as one of the top transfectants and produced up to about 60 mg/ml in shake flasks and about 200 mg/L in a fermentor.

### EXAMPLE 3

### Development and Characterization of Additional Transfected Clones and Cell Lines: Sequential Transfections

This example describes a further increase in expression and production of polypeptides, for example, human engineered anti-Ep-CAM immunoglobulins, through a second transfection of an exemplary cell line with a second multi-transcription unit vector.

Two additional vectors (as described in Example 1) were employed that were identical to pING1937 each comprising two transcription units, with a low risk human engineered ING-1 light chain gene and a low risk human engineered heavy chain gene, except that they have either a his gene encoding histidinol resistance (PING1957) or a gpt gene encoding mycophenolic acid resistance (pING1959). The development of an ING-1 immunoglobulin producing CHO cell line, Clone 259, is described. Clone 259 was developed by transfecting Clone 146 cells (as described in Example 1) with the his expression vector pING1957. The development of another ING-1 immunoglobulin producing CHO cell line, Clone 373, is also described. Clone 373 was developed by transfecting a subclone of Clone 146, Clone 146.3 cells, with the gpt expression vector pING1959.

A. Transfection of Clone 146 with pING1957 and Development of Clone 259

Clone 146 was transfected with pING1957 in serum-free medium (Ex-Cell 301). First, 650 clones were screened from 2 transfections in 96 well plates. Then, 142 clones were selected from the 96 well plates and then screened from 24 well plates. Finally, 31 clones were selected from the 24 well plates and screened in shake flasks. The results for top producers in Ex-Cell 301 media without FBS of antibody as measured by HPLC demonstrated that the top producers expressed antibody at greater than 2 times higher levels than Clone 146. For example, the top producer, Clone 146.2-259, expressed 172 μg/ml and 192 μg/ml in two different tests.

Clone 146.2-259 was subcloned in Ex-Cell 301 medium and screened in a 24 well format. The top subclones were further selected based on shake flask productivity in Ex-Cell 301 serum-free medium. Shake flask results for top producers in Ex-Cell 301 without FBS of expression of immunoglobulin polypeptide as measured by HPLC demonstrated that the top 259 subclones expressed antibody at about 1.5 to 2 times higher levels (e.g., from about 229 μg/ml to about 271 μg/ml) than the parent Clone 259 (e.g., about 116 μg/ml).

B. Transfection of SubClone of Clone 146, Clone 146.3, with pING1959 and Development of Clone 373

Clone 146, the initial pING1937 G418-resistant transfectant was also subjected to subcloning in Ex-Cell medium and one subclone, 146.3, secreted ˜121 μg/ml compared to ˜65 μg/ml for Clone 146 in Ex-Cell medium.

Since Clone 146.3 secreted at a relatively high level for a single transfection, it was therefore subjected to transfection with pING1959. Serum-free medium (Ex-Cell 301) adapted Clone 146.3 cells were transfected with pING1959 (same as pING1937 except for mycophenolic acid resistance as described in Example 1), plated in Ham's F12 with 5% FBS/mycophenolic acid and xanthene for selection. First, 520 clones were screened from 2 transfections in 96 well plates. Then 106 clones were selected from 24 well plates and screened. Finally, 26 clones were selected from the 24 well plates and screened in shake flasks. The sequential transfection of Clone 146.3 with pING1959 resulted in the selection of Clone 373 which expressed ˜225 and ˜257 μg/ml immunoglobulin polypeptide as determined by the shake flask results in Ex-Cell 301 medium.

### EXAMPLE 4

### Development and Characterization of Additional Transfected Clones and Cell Lines: Further Sequential Transfections

This example describes expression and production by a third sequential transfection of an exemplary cell line with a third multi-transcription unit vector, resulting in clones and cell lines that express increased levels of polypeptide production, including anti-Ep-CAM immunoglobulin production.

Clone 373 as described in Example 3 was chosen for additional studies and was further subjected to another sequential transfection using pING1957 (same as pING1937 except for histidinol resistance) in serum-free medium (Ex-Cell 301) and plated in Ex-Cell 301 supplemented with FBS and histidinol. Once the clones were selected, they were maintained with G418, MPA/xanthine/histidinol. First, 160 clones were screened from 2 transfections in 96 well plates. Then, 48 clones were selected from the 96 well plates and screened in 24 well plates. Finally, 12 clones were selected from the 24 well plates and screened in shake flasks. Results for shake flask tests in Ex-Cell 301 yielded 8 top producing clones.

The top producing clones displayed an expression level ranging from about 310 to about 370 μg/ml, including Clone 132 which had an expression level of about 317 μg/ml.

### EXAMPLE 5

### Development and Characterization of Additional Transfected Clones and Cell Lines with Multiple Transcription

The expression vectors, pING1959 (FIG. 9) and pING1965 (FIG. 13) containing one copy of each of the human engineered ING-1 light and heavy chain genes (pING1959, two gene vector) or two copies of each of the human engineered ING-1 light and heavy chain genes (pING1965, four gene vector) were transfected into CHO-K1 cells. CHO-K1 cells adapted to suspension growth in Ex-Cell 301 medium were electroporated with 40 μg of each linearized vector. After a recovery period of 2 days without selective agent, cells were plated in 96 well plates containing Ham's F12 medium supplemented with 5% FBS, mycophenolic acid and xanthine. A total of 300 and 255 clones were screened in 96 well plates for transfections with pING1959 and pING1965, respectively. For the pING1959 transfections, the top 18 clones were transferred to 24 well plates containing Ex-Cell 301 medium supplemented with 1% FBS. For the pING1965 transfections, the top 40 clones, were transferred to 24 well plates containing Ex-Cell 301 medium supplemented with 1% FBS. All 18 clones from the pING1959 transfection were next transferred to shake flasks containing Ex-Cell 301 medium supplemented with 1% FBS and evaluated for productivity. The top two producers, Clones 53 and 157 secreted ˜116 and ˜133 μg/ml, respectively in the presence of 1% FBS. In ExCell 301 medium without FBS supplementation Clones 53 and 157 secreted
—117 and ˜121 μg/ml, respectively. For the pING1965 transfection, the top 8 clones were transferred to shake flasks and evaluated for productivity. The top producer, Clone 17, secreted ˜216 μg/ml in ExCell 301 medium supplemented with 1% FBS and ˜214 μg/ml in ExCell 301 medium without FBS supplementation. Accordingly, a cell line transfected with a four gene vector (pING1965) with two copies of each of the human engineered light and heavy chain genes did produce approximately twice as much immunoglobulin polypeptides as cell lines transfected with a two gene vector (pING1959) with one copy of each of the human engineered ING-1 light and heavy chain genes.

### EXAMPLE 6

### Evaluation of Binding Activity of Immunoglobulin Polypeptides

Vectors constructed according to Example 1 encoding human engineered ING-1 light and heavy chain genes, for example, pING1937 (low risk human engineered ING-1) and pING1944 (low plus moderate risk human engineered ING-1) were linearized by XbaI and used to transfect serum-free adapted CHO-K1 followed by selection of G418-resistant transfectants. Protein was purified from shake flask culture supernatants by passage over a protein A column. To evaluate the binding activity of the produced immunoglobulin polypeptides, competition binding assays with the human carcinoma cell line HT-29 were performed. This colorectal carcinoma cell line expresses a molecule known as Ep-CAM on its surface. Ep-CAM is recognized by immunoglobulin polypeptides having the antigen binding specificity of the mouse-human chimeric ING-1 antibody produced by cell line HB9812 as produced by ATCC.

HT-29 cells were grown to confluency (˜2×10⁵ cells/well) in 96 well plates. Mouse-human chimeric ING-1 was labeled with Na¹²⁵I (Iodo-gen®, Pierce). The competition conditions included in a 100 μl assay volume, 0.1 nM labeled mouse-human chimeric ING-1,2-fold serial dilutions of unlabeled immunoglobulin polypeptides, for example, as produced by cells transfected with pING1937 and pING1944. The labeled and unlabeled immunoglobulin polypeptides were incubated with HT-29 cells at 4° C. for 5 hours followed by washing. Labeled cells were removed with NaOH and counted. Data analysis was performed using Ligand, [Munson and Redbard, Analytical Brochure 107: 220-239 (1980)].

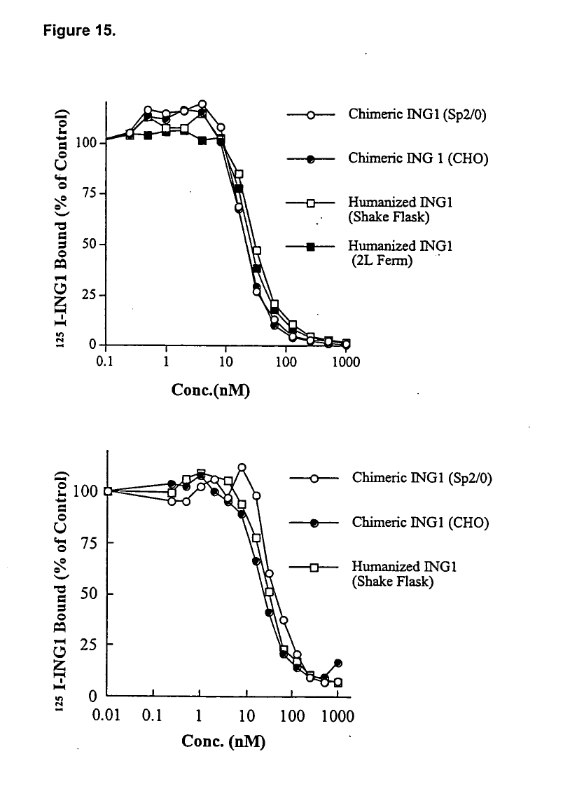

Results for competition binding assays using immunoglobulin polypeptides obtained from transfection with the pING1937 (low risk) vector are shown in FIG. 15. The affinities for the mouse-human chimeric ING-1, containing an un-modified ING-1 murine variable domain, and the human engineered ING-1, with its variable domain modified at low risk positions, showed very similar affinities (2-5 nM) (FIG. 15).

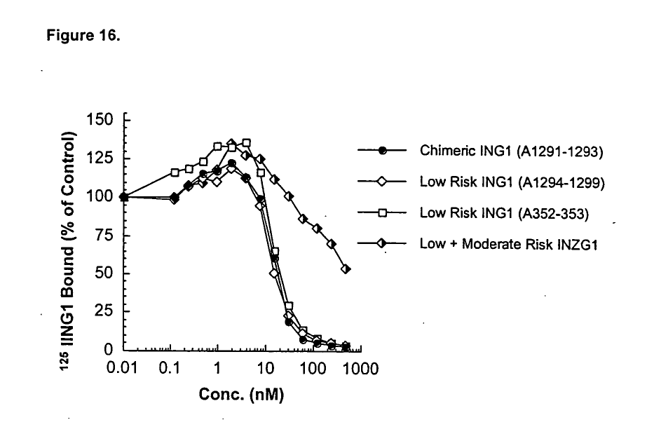

Human engineered ING-1 modified at the low risk plus moderate risk positions was also evaluated using competition binding assays. Results with the human engineered ING-1 purified from pING1944 transfected cell culture supernatant are shown in FIG. 16. No differences in binding between the mouse-human chimeric and the human engineered (low risk) ING-1 was observed. The human engineered (low plus moderate risk) ING-1 obtained from transfection with the vector pING1944 showed a reduced competition binding activity as shown in FIG. 16.

In order to examine the contribution of the light and heavy chain moderate risk changes on ING-1 binding activity, immunoglobulin polypeptides were expressed from vectors constructed with either the combination of a low risk light chain with a low plus moderate risk heavy chain or a low risk heavy chain with a low plus moderate risk light chain. Vectors containing both modified ING-1 light chain plus heavy chain were used to transfect serum-free medium-adapted CHO-K1 cells. Approximately 100 clones were screened in microtiter plates to select the top 8 to 10 clones for shake flask evaluation. For production purposes, the best producers were grown in shake flasks and modified ING-1 IgG's were purified on a protein A column followed by concentration determination by A₂₈₀.

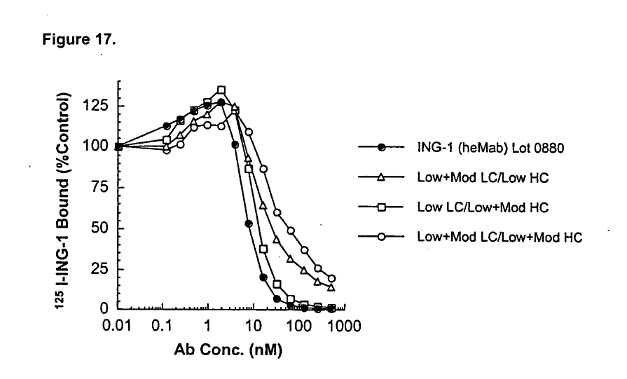

Competition binding assays, employing iodinated human engineered (low risk) ING-1 and Ep-CAM expressing HT-29 cells, were used to characterize modified ING-1 immunoglobulin polypeptides. Exemplary results are shown in FIG. 17. Moderate risk changes made to the light chain appeared to have the greatest impact on binding of the modified ING-1 antibodies tested. Moderate risk changes made to the heavy chain also appeared to effect binding, but to a lesser extent than the light chain changes. The results suggested that the effects observed with individual chains were additive.

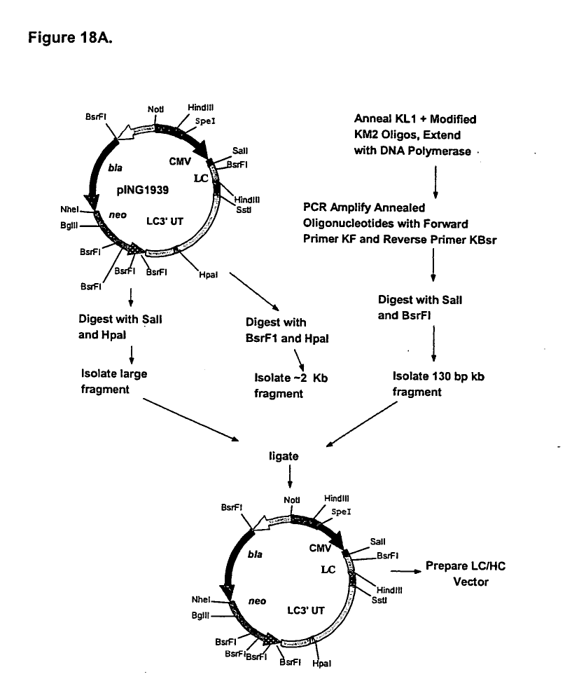

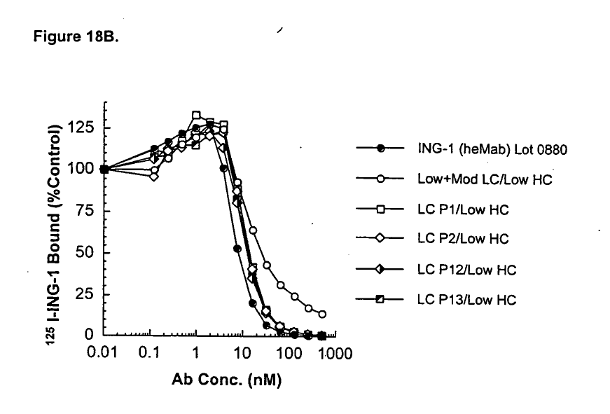

Moderate risk changes include changes involving prolines (see, e.g., FIGS. 5 and 7). The low plus moderate risk ING-1 light chain has 3 prolines introduced within Framework 1 [amino acids 1-59 (SEQ ID NO: 8)], and the low plus moderate risk ING-1 heavy chain has 1 proline removed within framework 1 [amino acids 1-57 (SEQ ID NO: 21)]. To examine the effects of proline changes in greater detail, low risk human engineered ING-1 light chain variable regions were constructed with prolines substituted in the low risk human engineered variable regions either one at a time [e.g. Proline 1 (P1) (SEQ ID NOS: 34 and 35), Proline 2 (P2) (SEQ ID NOS: 36 and 37), Proline 3 (P3) (SEQ ID NOS: 38 and 39) or combinations of prolines P1P2 (SEQ ID NOS: 40 and 41), P1P3 (SEQ ID NOS: 42 and 43), P2P3 (SEQ ID NOS: 44 and 45)] of the light chain and/or P1 of the heavy chain. As is shown in FIG. 13, the total of 3 amino acid positions in the low risk light chain that were changed to proline are within Framework 1. Therefore, all combinations of prolines were incorporated by using two overlapping oligonucleotides. KL1 remained unchanged, and was as described in Example 6 for the construction of the low risk ING-1 light chain vector pING1933. The second oligonucleotide used was one of 6 variations of the oligonucleotide KM2 described in Example 6 for the construction of the low plus moderate risk ING-1 light chain vector pING1939. Which variation of KM2 chosen depended upon which combinations of prolines were to be introduced into the low risk ING-1 light chain sequence. A low risk light chain variable region was modified with the first moderate risk proline (P1) substituted for the alanine at position 8 of the low risk ING-1 light chain (SEQ ID NO: 35). A low risk variable region was further modified with the second moderate risk proline (P2) substituted for the leucine at position 15 of the low risk ING-1 light chain (SEQ ID NO: 41). A low risk variable region was further modified with the third moderate risk proline (P3) substituted for the serine at position 18 (SEQ ID NO: 39). By employing one of 6 variations of the KM2 oligonculeotide (SEQ ID NO: 17), each praline residue was first changed separately P1 (SEQ ID NO: 46), P2 (SEQ ID NO: 47), P3 (SEQ ID NO: 48), and then in pairs as P1P2 (SEQ ID NO: 49), P1P3 (SEQ ID NO: 50) or P2P3 (SEQ ID NO: 51). The cloning strategy employed to construct expression vectors encoding various ING-1 light chains with different combinations of moderate risk proline residues incorporated into the low risk human engineered ING-1 light chain is shown in FIG. 18A. Subsequent to annealing the modified KM2 variant with the unmodified KL1 oligonucleotide, the annealling reaction was extended with DNA polymerase followed by amplification by PCR employing ING-1 light chain forward primer KF and reverse primer KBsr (SEQ ID NO: 52). The resultant product was then digested with SalI and BsrF1, followed by purification of the resultant 130 base pair fragment corresponding to low risk ING-1 light chain framework 1 region modified by the introduction of proline residues at one of three positions or in various combinations. The vector pING1939 was then digested with SalI and HpaI followed by purification of the large linear vector fragment. The vector pING1939 in a separate reaction was also digested with BsrF1 and HpaI followed by purification at the 2 kb fragment. These light chains, modified at various proline positions, were then expressed with the low risk heavy chain.

A three way ligation was performed in which the HpaI end of the pING1939 linear vector was first ligated with the HpaI end of the 2 kb fragment comprising the ING-1 light chain sequence minus the framework 1 region sequence. The full length ING-1 light chain was re-constructed when the BsrF1 end of the 130 bp proline modified fragment ligated to the BsrF1 end of the 2 kb fragment comprising the ING-1 light chain minus the framework 1 region. The vector was then closed when SalI end of the 130 bp proline modified framework 1 region fragment ligates to the SalI end of the pING1939 linear vector.

Competition binding assays, again employing iodinated human engineered (low risk) ING-1 and Ep-CAM expressing HT-29 cells, were used to determine the effect of prolines on binding activity. Results for modified ING-1 with a low risk heavy chain in combination with a light chain modified at P1, P2, P12, or P13 are shown in FIG. 18B. When compared with each other changes at any one or a combination of two, of the tested prolines, have a similar effect. Changing all three positions to prolines has the greatest effect.

### EXAMPLE 7

### Cloning and Expression of Soluble Human EP-CAM and EP-CAM Binding Assays

Competition binding assays with Ep-CAM-expressing cells such as HT-29 cells as described in Example 1 above require the use of isotopes and the maintenance and growth of cells for each assay, with the potential variability as with any cell-based assay. A direct binding ELISA assay with soluble Ep-CAM was also developed and used to evaluate the binding activity of immunoglobulin polypeptides produced by transfected cells according to the invention. For these assays, soluble human Ep-CAM (SEQ ID NOS: 53 and 54) was cloned and expressed. Immunoglobulin polypeptides used included human engineered (low risk) ING-1 from either 2 L or 500 L fermentor runs and soluble sEp-CAM from shake flasks or 2 L fermentors purified by ING-1 affinity chromatography.

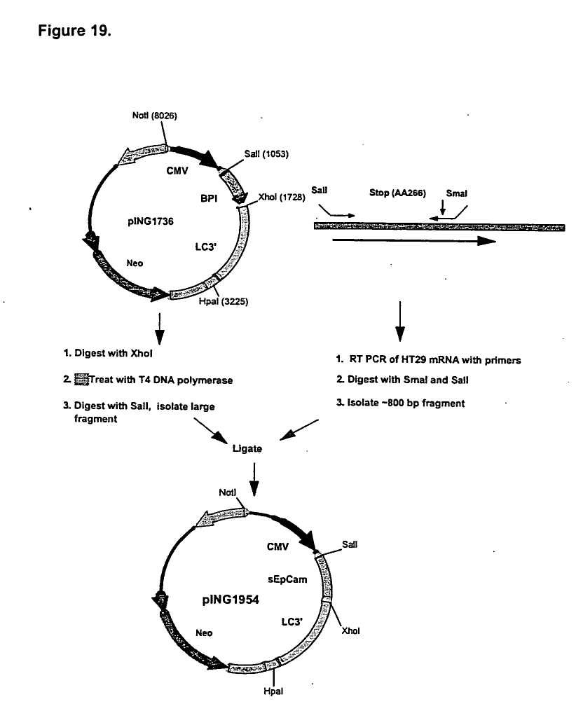

Soluble Ep-CAM has previously been expressed in insect cells. For expression in and secretion from CHO-K1 cells, truncated Ep-CAM (sEp-CAM) was cloned into an expression vector with the CMV promoter and the neo gene encoding for G418-resistant. The cloning strategy is outlined in FIG. 19. The vector pING1736 (described in Example 1 above) was digested with XhoI, followed by treatment with T4 DNA polymerase to blunt end. The blunt ended linear vector was then digested with SalI and the large fragment was isolated. The Ep-CAM gene was obtained from HT-29 mRNA using a RT PCR reaction which incorporated a 5′ SalI site, a 3′ SmaI site, and truncated the Ep-CAM gene by introducing a stop codon at amino acid position 266 (SEQ ID NOS: 53 and 54). Without that stop codon, the Ep-CAM sequence comprises 314 amino acids as shown in SEQ ID NOS: 55 and 56 (to clone this full-length Ep-CAM sequence, two primers may be used, an Ep-CAM forward primer (SEQ ID NO:57) and an Ep-CAM reverse primer (SEQ ID NO: 59)). For the RT PCR, two primers were used, an Ep-CAM forward primer (SEQ ID NO: 57) and an Ep-CAM reverse primer (SEQ ID NO: 58). The RT PCR reaction was digested with SmaI and SalI and the resultant 800 base pair fragment was purified and ligated with the digested pING1736 large fragment to produce vector pING1954.

Serum-adapted CHO-K1 cells were then transfected with linearized pING1954. Transfectants were then selected in Ex-Cell 301 with 2% FBS. Screening was performed in 24 well plates, and shake flask formats by direct ELISA. Detection was performed with peroxidase-labeled goat anti-human IgG. 150 clones, adapted to grow without FBS were transferred to 24 well plates. The cells were grown to extinction and 50 μl supernatant was used to pre-coat the plates. The top 6 clones were then transferred to shake flasks. The productivity for Clone 51 was subsequently estimated to be about 20 to 35 mg/L in both shake flasks and 2 liter fermentors.

Western analysis was employed to confirm that ELISA signal corresponded to specific proteins. Multiple bands were subsequently observed with the Ep-CAM supernatant. These multimers are also observed with purified Ep-CAM and were not an artifact of running crude culture supernatant, and therefore did not adversely impact the use of supernatants in an ELISA based direct assay. Moreover, the lack of detection on a reduced gel is consistent with the non-linear nature of the known Ep-CAM epitope structure recognized by immunoglobulin polypeptides with antigen binding like ING-1.

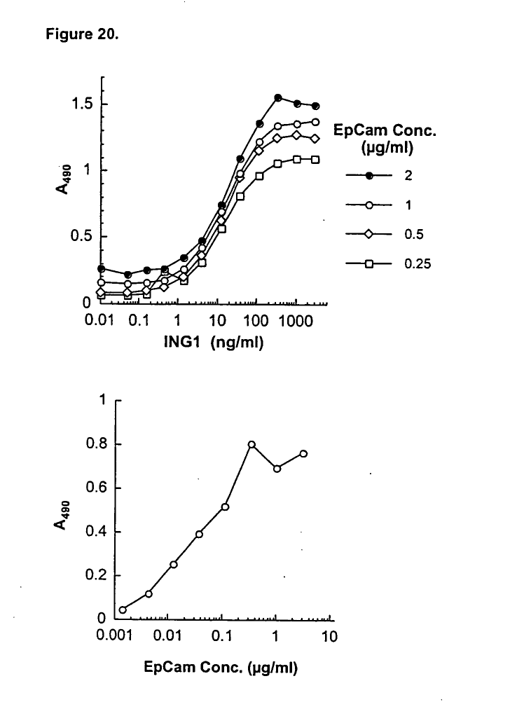

Immulon II plates were precoated with the sEp-CAM antigen. The sEp-CAM antigen was first diluted in a PBS coating solution to immobilize it to the microplate. sEp-CAM test concentrations ranging from 0.25 to 20 μg/ml were added at 50 μl/well and incubated at 4° C. overnight. The plates were then washed 3 times with PBS-0.05% Tween. Blocking was performed by adding in PBS-0.05% Tween 1% BSA followed by a 30 minute incubation at 37° C. Dilutions of immunoglobulin polypeptides were prepared in PBS-0.05% Tween 1% BSA solution. 2- or 3-fold serial dilutions were prepared and added (100 μl/well) in duplicate or triplicate. After a 90 minute incubation at 37° C., the microplate was washed 3 times with PBS-0.05% Tween. For signal development, goat anti-human IgG (gamma- or Fc-specific) secondary antibody conjugated to peroxidase was added to each well and incubated for 60 minutes at 37° C. followed by addition of OPD at 0.4 mg/ml in citrate buffer plus 0.012% H₂O₂. After 5-10 minutes at room temperature the assay was stopped by the addition 100 μl 1M H₂SO₄ and the plates were read at 490 nm. Both goat anti-human IgG (gamma-specific) and goat anti-human IgG (Fc-specific) antibodies have been employed. Results of the direct binding ELISA for human engineered (low risk) ING-1 on soluble Ep-CAM is shown in FIG. 20.

### EXAMPLE 8

### Purification of Immunoglobulin Polypeptides from Cultured Cell Lines

A process for the purification of immunoglobulins polypeptides from vectors and all lines according to the invention was designed. The process is described with reference to a human engineered (low risk) anti-Ep-CAM antibody from Clone 146 as described in Example 2B that was produced in a fed batch process using conventional stirred tank fermentors. Briefly, the process was as follows. Cells were removed by filtration after termination. The filtrate was loaded in multiple passes onto a Protein A column. The column was washed and then the expressed and secreted immunoglobulin polypeptides were eluted from the column. For preparation of antibody product, the Protein A pool was held at a low pH (pH 3 for a minimum of 30 minutes and a maximum of one hour) as a viral inactivation step. An adsorptive cation exchange step was next used to further purify the product. The eluate from the adsorptive separation column was passed through a virus retaining filter to provide further clearance of potential viral particles. The filtrate was further purified by passing through an anion exchange column in which the product does not bind. Finally, the purification process concluded by transferring the product into the formulation buffer through diafiltration. The retentate was adjusted to a protein concentration of 5 mg/mL and a stabilizer was added. The process is described in greater detail below.

A frozen aliquot of Clone 146 cells (see Example 2B above) were thawed rapidly and suspended in selective growth medium in a spinner flask. When the cell count reached approximately 0.5-1.5×10⁶ cells/mL, the cells were transferred to a larger spinner flask. The cells were expanded through successive transfers, each time when the cell density reached approximately 0.5×10⁶ to 1.5×10⁶ cells/mL, and each time in selective medium. The cell culture was further expanded in medium without selection in a 130 L fermentor vessel and was then transferred into a 500 L production fermentor. The temperature of the 500 L production fermentor, was maintained at 37° C. initially and then was dropped to 33° C. once the cells reach a specified concentration. Dissolved oxygen was controlled at ≧5% saturation. Harvest was performed before the cell viability fell below 50%, which was approximately nine to ten days.

Upon termination of cell culture production in the fermentor, the suspension was passed through filters to remove the cells. The filtered cell culture fluid was loaded on a Protein A column that had been equilibrated with 10 mM sodium phosphate, 150 mM sodium chloride, pH 7.0. The antibody produced by the Clone 146 cells was captured by selective adsorption while most of the protein and non-protein impurities flow through with the fluid. The column was washed with equilibration buffer (10 mM sodium phosphate, 150 mM sodium chloride, pH 7.0), and then eluted with 20 mM sodium citrate, pH 3.0. As a viral inactivation step, the Protein A pool was allowed to sit 30 to 60 minutes at pH 3.

The pH of the Viral Inactivated Protein A pool was then adjusted to 5.0 with 2.0 M TRIS base and the pool was filtered through a 0.2 micron filter. The pool was adjusted in conductivity to within ±5% of the SP Sepharose XL column equilibration buffer (20 mM sodium acetate, 50 mM sodium chloride, pH 5.0) and then applied to the SP Sepharose XL column pre-equilibrated in the same buffer. The antibody bound to the SP Sepharose XL column while impurities were eluted with the flow-through. The column was washed with equilibration buffer (20 mM sodium acetate, 50 mM sodium chloride, pH 5.0) and the antibody was eluted with 20 mM sodium acetate, 175 mM sodium chloride, pH 5.0.

The eluate from the SP Sepharose XL column was filtered first through a 0.2 micron filter, which served as a pre-filter for the finer porosity Viresolve filter that followed. This step was implemented specifically for viral particle removal.

The Viresolve filtrate was passed through a 0.2 micron filter prior to loading on a Q Sepharose column that had been equilibrated with 20 mM TRIS, 175 mM sodium chloride, pH 8.0. This column was a purification step in which the antibody did not bind to the anionic media but passed with the flow through while remaining impurities were captured.

The eluate from the Q Sepharose column was filtered through a 0.2 micron filter, then concentrated by ultrafiltration to greater than 5 mg/mL. Diafiltration was then initiated to exchange product into the formulation buffer (20 mM sodium phosphate, 0.15 M sodium chloride, pH 7.2 or pH 6.5). After diafiltration, the retentate was diluted to 5 mg/mL with formulation buffer, and polysorbate 80 is added to 0.005% as a stabilizer. The formulated human engineered anti-Ep-CAM antibody was then filtered through a 0.2 micron filter into sterile containers and used in various assays and studies as described herein.

### EXAMPLE 9

### In Vitro Activity Studies

Human engineered anti-Ep-CAM antibodies as described herein were evaluated for their in vitro activity in ADCC (antibody-dependent cellular cytotoxicity) and CDC (complement dependent cytotoxicity) studies. Briefly summarized, in assays for such in vitro activities, the human engineered anti-Ep-CAM antibody produced from Clone 146 (see Example 2B above) caused concentration-dependent lysis of BT-20 breast, MCF-7 breast, HT-29 colon and CACO-2 colon tumor cells, with maximum cytolysis at approximately 1 μg/mL. ADCC activity against breast and colon tumor cells, with maximal lysis approaching 100% against one cell line was demonstrated with this anti-Ep-CAM antibody as described in more detail below.

A. Antibody-Dependent Cellular Cytotoxicity

Target cells for antibody-dependent cellular cytotoxicity lysis assays were cultured in DME/F12 supplemented with 10% fetal bovine serum (Hyclone, Logan, Utah). For labeling, cells were harvested with trypsin-EDTA, resuspended in RPMI 1640 at 5×10⁶/mL (1-2 mL) and incubated with 100 μCi/mL ⁵¹Cr (NEN, Boston, Mass.) for 45-60 minutes at 37° C. Cells were washed twice with RPMI 1640 and resuspended in appropriate medium before use.

ADCC assays were performed with peripheral blood mononuclear cells (PBMC) prepared from blood obtained from healthy volunteers using acid citrate dextrose as an anticoagulant. Sources included blood collected in Vacutainer collection tubes (Becton Dickinson, Franklin Lakes, N.J.), buffy coat cells obtained from the blood bank (American Red Cross Blood Services, Oakland, Calif.), and lymphapheresis cells (HemaCare, Sherman Oaks, Calif.). PBMC were isolated on a Ficoll-Paque (Amersham Pharmacia Biotech, Upsala, Sweden) step gradient and suspended in RPMI 1640 supplemented with 10% normal human AB serum (ABS, Sigma, St. Louis, Mo.). PBMC (8×10⁵) were mixed with labeled target cells (10⁴) and varying concentrations of the human engineered anti-Ep-CAM antibody, diluted in RPMI 1640 plus 10% ABS, in round bottomed 96-well assay plates. The plates were centrifuged for 1 minute at 250×g then incubated at 37° C. After 4 hours, the plates were centrifuged for 5 minutes at 550×g and supernatant medium was collected with a Skantron harvestor.

PBMC from four separate donors and ⁵¹Cr-labeled human tumor target cells (80:1 ratio) were incubated with increasing concentrations of ING-1. ING-1 caused a concentration-dependent lysis of BT-20 breast tumor cells (˜50-70% lysis at ˜1 μg/ml), MCF-7 breast tumor cells (˜60-90% lysis at ˜1 μg/ml), HT-29 colon tumor cells (˜85-100% lysis at ˜1 μg/ml) and CACO-2 colon tumor cells (˜60-95% lysis at ˜1 μg/ml). Although maximal killing was observed at approximately 1 μg/mL, as much as 50% lysis was evident at concentrations as low as 10 ng/mL. In other experiments, this human engineered anti-Ep-CAM antibody caused similar levels of lysis of non small cell lung (NCI-H1568), prostate (PC-3), and pancreatic (HPAF-II) tumor cells.

B. Complement Mediated Cytotoxicity

CDC assays were performed with pooled human serum collected from four healthy volunteers. Labeled target cells were suspended in RPMI 1640 at 4×10⁵ cells/mL. Target cells (2×10⁴) were mixed with serum and varying concentrations of the human engineered anti-Ep-CAM antibody, diluted in RPMI 1640, in round bottomed 96-well microtiter plates. Assay plates were incubated at 37° C. for 3 hours, centrifuged at 550×g for 5 min and the supernatant liquid was collected with a Skantron harvestor. Percent lysis was calculated by the equation: % Lysis=Experimental CPM−Spontaneous CPM/Maximum CPM−Spontaneous CPM where Spontaneous CPM was determined from wells containing no antibody and Maximum CPM was determined from wells where target cells were lysed with 1 M HCl.

In order to determine if this human engineered anti-Ep-CAM antibody mediated CDC, ⁵¹Cr-labeled HT-29 colon tumors cells served as the target and were incubated with different amounts of serum. The serum concentrations tested included 6.25%, 12.5%, 25%, and 50%. The ability of increasing concentrations of antibody to lyse the tumor cells at these various serum concentrations was measured as a release of ⁵¹Cr into the supernatant. This human engineered anti-Ep-CAM antibody caused dose-dependent lysis of the tumors cells with maximal killing occurring at approximately 1 μg/mL. At an antibody concentration of 1 μg/ml and a serum concentration of 6.25%, less than 5% of the target cells were lysed. At concentrations of serum at 12.5% and higher, as much as 20% cell lysis occurred at an antibody concentration of 1 μg/ml.

### EXAMPLE 10

### Pharmacokinetic Studies and In Vivo Activity Studies

Human engineered anti-Ep-CAM antibodies as described herein were evaluated in pharmacokinetic studies in animals with and without tumors, and for their in vivo activity in a variety of animal tumor models as described in more detail below.

A. Pharmacokinetic Studies

For pharmacokinetic studies of the human engineered anti-Ep-CAM antibody produced by Clone 146 as described in Example 2B above, two animal models were employed using male CD® rats (Charles River, Hollister, Calif.) weighing 280-320 grams, or male athymic nude mice (NCR nu/nu, Simonsen, Gilroy, Calif.) weighing 20-30 grams.

The pharmacokinetic data were entered on an Alpha 3000, model 600, computer (Compaq Corporation, Maynard, Tex.), and analyzed using a validated software system. Data of individual animals were fitted by nonlinear least squares analysis using the pharmacokinetic bi-exponential disposition function to describe the change in antibody concentration with time, with the inverse of the square of the model concentration as the weighting. The curve fits yielded four primary pharmacokinetic parameters: volume of distribution of the central compartment, the alpha half-life, beta half-life, and the coefficient to the beta half-life. Secondary pharmacokinetic parameters were calculated from the primary parameters [Gibaldi and Perrier, Multicompartment models. In: Pharmacokinetics, 2^{nd} edition. Marcel Dekker Inc, New York, pp. 45-111 (1982)].

Five male rats received 50 mg/kg antibody (50 mg/mL), and another five male rats received 0.5 mg/kg of antibody (0.5 mg/m]L), both intravenously (IV) via the tail vein. Blood was collected on days 0-91 via the retro-orbital sinus under methoxyflurane anesthesia. Six male rats received 5 mg/kg of antibody (5 mg/mL)/mL subcutaneously at a single location. Blood samples were collected from days 0-196 after dose injection. In all rat experiments, approximately 200 μl of blood was collected at each time point into microcentrifuge tubes containing sodium citrate. Plasma was extracted and stored at −70° C. until assayed.

Anti-Ep-CAM antibody was measured in rat plasma by ELISA. Microtiter plates were coated with the capture reagent, soluble Ep-CAM diluted to 0.25 μg/mL in phosphate buffer saline (PBS). The detection system consisted of alkaline phosphatase-conjugated goat anti-human IgG antibody (Zymed Laboratories, South San Francisco, Calif.) with p-nitrophenylphosphate as substrate. Color development was allowed to proceed for 1 hour at room temperature and then terminated with 100 μL of 1 N NaOH. The absorbance at 405 nm was determined for all wells using a Vmax Plate Reader (Molecular Devices, Menlo Park, Calif.). A standard curve was generated and samples were quantified by interpolation from the standard curve. Plasma standards were prepared by adding known amounts of antibody to plasma. These standards were used to calculate the proportion of antibody recovered by the assay in plasma. A linear regression of antibody concentration measured by ELISA versus added antibody concentration was performed, and the calculated slope was used as the fractional recovery. T plasma concentrations of antibody in the samples were then corrected for the recovery.

The decline in plasma concentration with time of IV administered anti-Ep-CAM antibody in the male rat animal model, an antigen negative species, was described by a bi-exponential pharmacokinetic disposition function. The alpha phase half-lives were approximately 6 and 13 hours for 0.5 and 50 mg/kg, respectively, while the beta phase half lives were approximately 18 and 17 days, respectively. The clearance was approximately 4.5 mL/day/kg at the two doses. Thus, the clearance of anti-Ep-CAM antibody was dose-independent over the dose range studied.

The plasma concentration-time profile did not reveal a change of kinetics at 10-14 days or later, signifying there was no host antibody production that altered antibody clearance.

After subcutaneous administration of 5.0 mg/kg of the antibody, plasma concentrations increased to a peak concentration of 21.5±0.7 μg/mL by 4.94±0.41 days. Thereafter, the plasma the antibody levels declined with a half-life of 16.7±0.8 days, similar to the beta half-life observed after IV administration. The bioavailability of subcutaneously administered the antibody relative to IV administered the antibody was calculated to be 57±4%. In one of the rats, the antibody plasma level declined rapidly after day 7, and was below detection by day 14. As a result of this observation, plasma from all rats was assayed for anti-human antibodies.

Antibodies to be administered in the rat were assayed by ELISA. Microtiter plates were coated with anti-Ep-CAM antibody to which rat plasma samples were added. The signaling system consisted of biotin-conjugated antibody to which was added alkaline phosphatase-conjugated streptavidin (Zymed, South San Francisco, Calif.) as the enzyme for the substrate p-nitrophenylphosphate. Standards of different concentrations of goat anti-human IgG (Sigma, St. Louis, Mo.) were assayed to convert the absorbance measurements of rat samples into goat anti-human IgG μg/mL equivalents. Antibodies were detected in the plasma of the one rat with altered clearance 21 and 70 days after injection, but not in the pre-dose sample. No detectable levels of anti-human antibody were measured in the other rats.

To assess the pharmacokinetics of the anti-Ep-CAM antibody when administered to mice without tumors, 13 mice that did not bear tumors received an intravenous bolus of 5 mg/kg (0.5 mg/mL) antibody. In mice without tumors, the decline in plasma concentration of antibody with time could be described by a bi-exponential pharmacokinetic disposition function. The average alpha phase half-life was 2.8±2.4 hours and the beta phase half-life was 10.1±0.5 days. The central compartment volume of distribution was 53±17 mL/kg, similar to plasma volume, and the clearance was 9.7±1.1 mL/day/kg.

The pharmacokinetic results, described above for rats and mice without tumors, demonstrated that the anti-Ep-CAM antibody reached adequate levels (>1 μg) for sufficient time. The results also demonstrated that the clearance profile of the antibody in nude mice without tumors, and in rats, was dose-independent and was similar to that of a typical native IgG1 without a specific host target site. The terminal half-lives of 10 days in nude mice without tumors and 20 days in rats for this human engineered anti-Ep-CAM antibody was similar to that previously reported for native IgG1 [Adams D O et al., Proc. Natl. Acad. Sci. USA 81:3506-3510 (1984) and Herlyn D M et al., Cancer Res. 40:717-721 (1980)].

To assess the pharmacokinetics of the anti-Ep-CAM antibody when administered to mice with Ep-CAM-positive tumors, thirteen male nude mice with HT-29 human colon tumors that averaged 195 mm³ received an intravenous bolus of 5 mg/kg (0.5 mg/mL) of this anti-Ep-CAM antibody. By 49 days post-dose, the average tumor volume was 2200 mm³.

In tumor-bearing mice, the plasma concentration of antibody declined with time and could be described by a bi-exponential pharmacokinetic disposition function for the first 14 days after dosing. The average alpha phase half-life was 1.9±0.7 hours and beta phase half-life was 5.7±0.4 days. The central compartment volume of distribution was 56±5 mL/kg and the clearance, based on the first 14 days, was 15.1±0.7 mL/day/kg. After 14 days, the plasma concentration of the human engineered anti-Ep-CAM antibody declined more rapidly, to near detection levels (5 ng/mL) on day 49, with an effective half-life of about 2 days.

The pharmacokinetics of the anti-Ep-CAM antibody in tumor-bearing mice contrasted with the experiments done with non-tumor bearing animals, signify that the presence of Ep-CAM-positive tumors results in more rapid clearance than was observed in tumor-negative mice. After three days, the increased clearance in mice with human tumors resulted in a nearly two-fold faster beta phase half-life compared to mice without human tumors. Furthermore, after 21 days, there was an ever increasing proportional decrease in the clearance rate for the mice with human tumors. Such a concentration-time profile is characteristic of a drug which is cleared by a slow, non-saturable elimination mechanism at high concentrations, and a faster saturable elimination mechanism at lower concentrations, apparently represented by human Ep-CAM in the tumors. The concentration-time profile of anti-Ep-CAM antibody in tumor-bearing mice was also characteristic of proteins which bind to specific host target sites of low capacity and high affinity [Johnson et al., J Immunol, 136: 4704-4713 (1986); Masucci, et al., Hybridoma 8: 507-516; Abdullah, et al., Cancer Immunol. Immunother 48; 517-524 (1999); Cheung et al., J. Clin. Invest. 81; 1122-1128 (1988); Gorter, et al., Lab. Invest. 74; 1039-1049 (1996)]. These pharmacokinetic signify a direct interaction of the antibody with human tumor cells in nude mice.

B. In Vivo Activity Studies

1. Establishment Models

For in vivo activity studies of the human engineered anti-Ep-CAM antibody produced by Clone 146, efficacy studies in a mouse xenograph model were performed with male athymic nude mice (NCR nu/nu, Simonsen Laboratories, Gilroy, Calif.), 20-25 g, which were maintained in a pathogen free facility. Each mouse received a subcutaneous injection of 3×10⁶ HT-29 colon tumor cells or 5×10⁶ PC-3 prostate cells in a flank region. After 24 hours, groups of 10 mice received the anti-Ep-CAM antibody at 0.1, 0.3 or 1.0 mg/kg (IV). Control mice received 1 mg/kg of human IgG1. Dosing was continued for 3 weeks, 2 doses per week. After tumors could be palpated, length and width measurements were obtained twice a week with microcalipers. Tumor volumes were calculated as L×W²/2. Differences in mean tumor volumes between groups were analyzed by a one-way analysis of variance with repeated measures. Post-hoc analysis was performed with Tukey's honest significant difference test.

The effect of the human engineered anti-Ep-CAM antibody on growth of HT-29 colon tumors in nude mice was measured. Antibody treatment 2 times per week (IV), for three weeks, resulted in a dose-dependent reduction in tumor size relative to control. An antibody dose of 1 mg/kg resulted in a 64% reduction in tumor size. Similar results were obtained in nude mice with PC-3 prostate xenografts. Antibody treatment, at 1 mg/kg, administered twice a week for 3 weeks, resulted in a 71% reduction in tumor volume.

For additional in vivo activity studies of the human engineered anti-Ep-CAM antibody produced by Clone 146, the ability of this antibody to inhibit the growth of human MCF-7 human breast tumors was studied in female athymic mice. Female athymic mice were injected with 3×10⁶ MCF-7 human breast tumor cells subcutaneously (SC) in 0.2 mL one day after implantation of a pellet containing 1.7 mg of β-estradiol. Twenty-four hours after inoculation with tumor cells, groups of 10 mice received 10, 30, or 100 mg/kg of antibody intraperitoncally (IP). A separate group of 10 mice received the same volume of vehicle as a control. Tumors in mice receiving this anti-Ep-CAM antibody at 10 mg/kg were 80% smaller than tumors in the vehicle treated group. Tumor volume in the mice receiving 30 mg/kg was reduced by 91% whereas there was a 68% reduction in tumor volume associated with 100 mg/kg of the antibody.

The ability of this antibody to inhibit growth of human PC-3 prostate cancer cells was also studied in female nude mice. Female nude mice were SC injected with PC-3 prostate cancer cells. Antibody was dosed at 1 mg/kg and 10 mg/kg (IV) two times a week for three weeks starting at Day 2. The antibody inhibited the outgrowth of tumors at 1 mg/kg. The effect of this antibody at 10 mg/kg was less than 1 mg/kg.

The ability of this antibody to inhibit growth of human Non-Small Cell Lung Cancer (NSCLC) cells was also studied in female nude mice. Female nude mice were injected subcutaneously (SC) with H1568 NSCLC cells. Antibody was dosed at 1 mg/kg (IV) two times a week for three weeks starting at Day 2. No effect of the antibody was observed on tumor outgrowth of H1568 NSCLC cells.

The ability of this antibody to inhibit growth of HPAII pancreatic tumor cells was also studied in female nude mice. Female nude mice were injected (SC) with HPAF-II pancreatic cancer cells. Antibody dosed at 1 mg/kg (IV) two times a week for three weeks starting at Day 2. No effect of the antibody was observed on tumor outgrowth of HPAF II pancreatic cells.

2. Established Models

The ability of this human engineered anti-Ep-CAM antibody to inhibit tumor growth was tested without much effect in several established tumor models. In one study, thirty nude mice received subcutaneous injections of 3×10⁶ HT-29 colon tumor cells/mouse on their right flanks, on Day 1. On Day 10, when the average tumor sizes reached 89 mm³, mice were randomly divided into 3 groups of 10 based on equal distribution of tumor sizes, and received their first dose of antibody. The animals were dosed twice a week for 3 weeks. One group received human IgG as a control at 1 mg/kg. The second group received 1 mg/kg (IV) of the anti-Ep-CAM antibody. The third group received 1 mg/kg of the anti-Ep-CAM antibody directly into the tumor. Mice receiving 100 mg/kg 5FU/LV served as positive controls. Intratumoral injections of this human engineered anti-Ep-CAM antibody provided no activity of tumor response in this established tumor model.

A 5FU/LV with antibody combination study was also conducted with the same HT-29 colon tumor cells. On Day 1, mice received a subcutaneous injection of 3×10⁶ HT-29 colon tumor cells on their right flanks. When tumors reached an average size of 100 mm³, mice received the human engineered anti-Ep-CAM antibody (IV) two times a week for three weeks. Some dose groups received the antibody and 5FU/LV combination treatment. In this combination study, the dose level of 5FU/LV was kept constant (100 mg/kg). The dose level of the human engineered anti-Ep-CAM antibody was varied among different combo groups. Control mice received either 5FU/LV or IgG treatment. Intravenous injections of the human engineered anti-Ep-CAM antibody alone provided no response in this established tumor model. In addition, in the combo-treatment groups, this antibody did not enhance the efficacy of 5FU/LV.

The ability of this antibody to inhibit tumor growth was also tested in established tumor models employing H1568 human NSCLC, HPAF II human pancreatic cancer, and prostate cancer cell line PC-3 tumor cell lines, as described above for the HT-29 colon tumor cell line. For the H1568 human NSCLC tumor cell line, the human engineered anti-Ep-CAM antibody was dosed at 1 mg/kg (IV) two times a week for three weeks starting at a tumor volume of 60 mm³. For the HPAF II human pancreatic cancer cell line the human engineered anti-Ep-CAM was dosed at 1 mg/kg (IV) two times a week for three weeks starting at a tumor volume of 100 mm³. For the prostate cancer cell line PC-3, the human engineered anti-Ep-CAM was dosed at 1 and 10 mg/kg (IV) two times a week for three weeks starting at a tumor volume of 100 mm³. No effect on tumor growth was observed for any of these cell lines employed in an established tumor model.

3. Metastatic Models

In order to study the ability of the antibody to effect metastasis, metastatic models were set up employing HT-29 colon, LS174T colon, MCF-7 breast, and human colorectal (Colo-205 GFP) cancer cell lines.

For the HT-29 colon cancer cell line metastatic model, nude mice on Day 1 received a tail vein injection of PBS containing 0.5 μg of recombinant human interleukin-1β. Five hours later, all mice received an IV injection of 3×10⁶ HT-29 colon cancer cells. Beginning 1 day after cell inoculation, mice received either human IgG (1 mg/kg, IV) or the human engineered anti-Ep-CAM antibody (1 mg/kg, IV) twice a week for three weeks. Additional animals received the same dosing with this antibody beginning on Day 5. A final group was treated with 5-FU/LV (100 mg/kg, IP) once a week for three weeks. Animals from all groups were sacrificed 8 weeks post tumor cell injection and necropsy was performed.

The results demonstrated a significant tumor reduction between the control (IgG treated) group and the anti-Ep-CAM antibody Day 2 treated group (p<0.005). A significant tumor reduction was also observed between the control group and the 5-FU/LV treated group (p<0.005). Overall, both treatments resulted in significant reduction of visible tumor nodules during necropsy and of nodules on lung surfaces. No statistical difference was observed between the control group and the anti-Ep-CAM antibody/Day 5 treated group. However, microscopic evaluation of the lungs showed a reduction in micrometastases in both anti-Ep-CAM antibody treatment groups.

An additional HT-29 colon cancer cell line metastatic model was studied. In this HT-29 metastasis study, four different antibody dose regimens were examined: a) 1 mg/kg, Day 2 (twice weekly, 3 weeks); b) 0.3 mg/kg, Day 2 (twice weekly, 3 weeks); c) 0.3 mg/kg, day 2 (once weekly, 3 weeks); and d) 30 mg/kg, Day 5 (twice weekly, 3 weeks). 1 mg/kg of IgG/day 2 was used as a negative control. 100 mg/kg 5FU/LV, both on Day 2 and on Day 5, were used as positive controls. The results demonstrated that the human engineered anti-Ep-CAM antibody significantly inhibited growth of tumor metastasis in 2 dose regimens: 1 mg/kg/Day 2 and 0.3 mg/kg/Day 2 (twice weekly, 3 weeks). Both treatments resulted in significant reduction of visible tumor nodules during necropsy and of nodules on lung surfaces. No statistical difference in nodule reduction was observed between the other 2 antibody regimens (0.3 mg/kg/Day2, once weekly, 3 weeks; and 30 mg/kg/Day 5, twice weekly, 3 weeks) and the negative control group. However, all 4 regimens with the human engineered anti-Ep-CAM antibody were effective in inhibiting formation of micrometastases, as demonstrated by microscopic examination of lung tissue sections.

For the LS174T colon cancer cell line metastatic model, on Day 1, nude mice received intrasplenic injection of 1×10⁶ LS174T cells, followed by spleen resection. Starting on Day 2, 8 mice received an IV dose of the anti-Ep-CAM antibody two times weekly for three weeks, 8 mice received IgG (control), and 8 mice received 5 FU/LV at 100 mg/mm². Mice monitored for three weeks and at the end all mice are sacrificed for necropsy.

All mice receiving 5 FU/LV died on Day 2. The anti-Ep-CAM antibody/Day 2 treated group observed a reduction in tumor burden compared with the IgG (control) treated group.

For the MCF-7 breast cancer cell line metastatic model, on Day 1, mice received an IV injection of 1×10⁶ MCF-7 cells via tail veins. (All mice were pre-implanted with slow-releasing estradiol pellets). Starting on Day 2 or Day 5, mice received the anti-Ep-CAM antibody (IV), twice weekly for three weeks. Mice were treated for 8 weeks and then sacrificed for necropsy.

The results showed that the anti-Ep-CAM antibody/Day 2 provided a survival advantage compared with the IgG (control) treated group. In addition, the anti-Ep-CAM antibody/Day 2 showed a significant reduction (p<0.005) in microscopic tumor nodules on lung surfaces.

The human colorectal cancer cell line Colo-205 GFP metastatic model, on Day 1 mice received orthotopic implantation on their cecal walls of small tumor pieces derived from colo-205 GFP cells. Starting on Day 2 or Day 9, mice received the anti-Ep-CAM antibody (IV), twice weekly for three weeks. Tumor growth was monitored throughout the study course. Areas of GFP expression throughout animal bodies were also examined via external green-fluorescence imaging. At the end of study (8 weeks), mice were sacrificed, GFP images of animals' open bodies were collected. GFP expression in 4 tissues (lung, spleen, liver, kidney) was analyzed. The results showed that the anti-Ep-CAM antibody treatment starting on both Day 2 and Day 5 significantly decreased tumor growth rate. The total areas of GFP expression (as represented by the sum of primary tumor size and tumor metastases) in mice receiving the anti-Ep-CAM antibody (both Day 2 and Day 5) were also significantly reduced.

### EXAMPLE 11

### Clinical Studies

Human engineered anti-Ep-CAM antibodies as described herein were evaluated in human studies as described in more detail below, including with an antibody comprising a heavy chain variable region having the amino acid sequence of SEQ ID NO: 19 and a light chain variable region having the amino acid sequence of SEQ ID NO: 6.

Three small Phase I studies were conducted to evaluate the safety, tolerability, pharmacokinetics, biodistribution and/or bioavailability of a human engineered anti-Ep-CAM antibody produced by Clone 146 as described in Example 2B.

A. Study 1

This first study was an open-label, multi-dose, Phase I, dose-escalating study to evaluate the safety, tolerability, and pharmacokinetics of an intravenously administered human engineered anti-Ep-CAM antibody in subjects with advanced adenocarcinomas, including adenocarcinoma of the ovary, breast, lung, prostate or gastrointestinal (GI) tract (e.g., colorectal (colon and/or rectal), pancreatic, gastric, esophageal).

Study objectives included the following: (1) to determine the maximum tolerated dose (MTD) of the first dose of the human engineered anti-Ep-CAM antibody; (2) to determine the quantitative and qualitative toxicity of the human engineered anti-Ep-CAM antibody administered as single intravenous (IV) infusions every 21 days for four doses; (3) to assess the pharmacokinetics of the human engineered anti-Ep-CAM antibody when administered on this schedule and to relate pharmacokinetics to drug effects (toxicity and activity) wherever possible; and (4) to preliminarily document any antitumor activity of the human engineered anti-Ep-CAM antibody.

This study was designed as an open-label, multi-dose, phase I, dose-escalating study to evaluate the safety, tolerability, pharmacokinetics, and biologic effects of the human engineered anti-Ep-CAM antibody in subjects with advanced adenocarcinomas. Subjects received the human engineered anti-Ep-CAM antibody as a single, one-hour intravenous infusion, which was repeated every 21 days. Subjects were continuously reassessed for retreatment and were followed for 21 days after the last dose. At least five dose groups were planned for this study and a minimum of three subjects were to be enrolled in each dose group. Subjects were assigned sequentially to the same dose group until all the subjects for that dose group had been enrolled. Only one subject was enrolled on any given day. Subjects were enrolled in the next higher dose group 21 days after the last subjects in the lower-dose group had been dosed and the Medical Monitor had reviewed the safety data for all the subjects and had not observed any dose-limiting toxicity (DLT). The MTD was defined as that dose preceding the dose at which ≧2 of 6 or ≧2 of 3 evaluable subjects experienced DLT during their first treatment cycle.

Safety was assessed by pretreatment, during-treatment and posttreatment physical examinations (including vital signs), clinical laboratory assessments (including blood chemistries, hepatic enzymes, bilirubin, amylase, lipase, hematology, prothrombin time/international normalized ratio [PT/INR], partial thromboplastin time [PTT] and urinalysis) and records of adverse clinical events. Plasma levels of study medication were assessed by serial monitoring of plasma. Subjects were screened for the presence of Ep-CAM positive micrometastases in the peripheral blood. The blood of subjects who were positive at baseline was monitored for micrometastases as the means for following their response. In addition, subjects were followed for tumor response by CT-scan, chest X-ray, MRI, tumor markers, or other appropriate methods. Subjects were screened and monitored for the development of human-antihuman antibodies (HAHA) as well as for anti-idiotypic antibodies (anti-id Ab). All 22 subjects entered into the study were closely and continually monitored for safety, tolerability, pharmacokinetic and tumor responses by frequent assessments of clinical signs and symptoms and other test results.

Subjects were to be included in the study if they met all of the following criteria: (1) subject had histologic or cytologic diagnosis of adenocarcinomas of the ovary, breast, lung, prostate or GI tract (colorectal, pancreatic, gastric, esophageal); (2) subject had an advanced adenocarcinoma that was either refractory to standard therapies or for which therapies that may potentially be of major benefit did not exist; (3) subject's disease status was evaluable or measurable; (4) subject had a performance status of 0 to 2 on the Eastern Cooperative Oncology Group (ECOG) scale; (5) subject had an estimated life expectancy of at least 12 weeks; and (6) subject had adequate hematologic, hepatic, renal, and pancreatic organ function. Five dose levels were to be evaluated by adding cohorts of three subjects at each of the following doses: 0.03 mg/kg, 0.1 mg/kg, 0.3 mg/kg or 1.0 mg/kg. Each dose was to be given as a one-hour IV infusion, which was to be repeated every three weeks for a total of four doses per subject.

Safety variables for evaluation included: recording adverse clinical events; clinical laboratory assessments, including blood chemistries, hematology, urinalysis, hepatic enzymes, bilirubin, amylase, lipase, prothrombin time/international normalized ratio (PT/INR), and partial thromboplastin time (PTT); pretreatment and posttreatment physical examinations, including vital signs and electrocardiogram (ECG); human anti-human antibody (HAHA) and anti-idiotypic antibodies (anti-id Ab); pharmacokinetic blood sampling to establish possible correlations between total plasma concentrations of human engineered anti-Ep-CAM antibody and toxicity; concomitant medications; toxicity rating. Efficacy variables for evaluation included: tumor measurements by CT-scan, chest X-ray, MRI, tumor markers or other appropriate methods to determine complete response (CR), partial response (PR), stable/no response, or progression; screening for the presence of Ep-CAM positive micrometastases in the peripheral blood; performance status using the ECOG scale.

Data were analyzed for safety, tolerability, preliminary efficacy, and pharmacokinetics. A detailed description of subject disposition was provided for all subjects. Safety analyses were performed on data from all subjects who received any amount of the human engineered anti-Ep-CAM antibody. The MTD was determined using the data from subjects who were treated with at least one dose and were followed for at least one full cycle (3 weeks). All adverse events (AEs), drug-related AEs, and serious adverse events (SAEs) were coded using the COSTART dictionary and summarized by dose group, body system and severity, as well as by body system, preferred term, and severity. Laboratory values outside of the corresponding normal ranges were identified. All dosed subjects with data from a sufficient number of samples had the following pharmacokinetic parameters calculated from plasma concentrations of the human engineered anti-Ep-CAM antibody serum concentration, peak plasma drug concentration (Cₘₐₓ), area under the serum concentration versus the time curve (AUC), systemic clearance (CL), central volume of drug distribution (Vc) and half-life (t_{1/2}). Levels of human engineered anti-Ep-CAM antibody in plasma and levels of micrometastases in subjects for whom levels were available were described over time. To assess the preliminary clinical efficacy of the human engineered anti-Ep-CAM antibody, tabulations of mortality, tumor markers and tumor response were presented for each subject.

At baseline, 9 (41%) subjects had a performance status, per the ECOG scale, of 0 (fully active and able to carry on all predisease performance without restriction) and 10 (46%) subjects had an ECOG performance status of 1 (restricted in physically strenuous activity but ambulatory and able to carry out work of a light or sedentary nature). The remaining three subjects had an ECOG performance status of 2 (ambulatory and capable of all self care but unable to carry out any work activities). The mean time since diagnosis was 43.3 months (range, 8-139 months). For the majority (64%) of subjects, the site of primary disease was colorectal, followed by ovarian (14%). Prior cancer treatment consisted of chemo/immunotherapy for 100%, surgery for 91%, radiation for 41%, and hormone therapy for 14%. At the time of the study, 68% of the subjects exhibited disease progression or relapse of their cancer, and 32% had residual disease. The mean time elapsed between progression/relapse and receiving the first dose was 1.7 months (range, 0.1-5.1 months).

The results of this study indicated that the MTD for the human engineered anti-Ep-CAM antibody was 0.3 mg/kg when administered IV every three weeks. The DLT was reversible pancreatitis at 1 mg/kg. The average half-life of the human engineered anti-Ep-CAM antibody at the MTD is 31 hours. The intravenous administration of the human engineered anti-Ep-CAM antibody for treating subjects suffering from advanced adenocarcinomas was considered to be safe and well-tolerated in this study. Most of the subjects experienced grade 1 or 2 drug-related AEs. The highest degree of severity of any of the drug-related AEs in either the 0.03 or 0.1 mg/kg dose group was grade 1 or 2. Two of 12 subjects in the 0.3 mg/kg group reported a total of six grade 3 and one grade 4 drug-related AEs. One patient in the 1 mg/kg group had one grade 3 AE. Six subjects experienced a total of nine serious AEs. Only one of the serious AEs (pancreatitis) was evaluated as being definitely related to the study drug. All five deaths that occurred were attributed to progression of the underlying disease and were deemed unrelated to the study drug. Only two of seventeen evaluable subjects developed a low level HAHA response toward the variable region as described in Example 12. A formal efficacy analysis was not performed, however, one of fifteen evaluable subjects had stable disease at 12 weeks.

B. Study 2

This second study was an open-label, multiple-dose, pilot study to evaluate the safety, tolerability, pharmacokinetics and biodistribution of an intravenously administered human engineered anti-Ep-CAM antibody in subjects with advanced adenocarcinomas, including advanced adenocarcinomas of the lung, colon, pancreas or prostrate. The objectives of this study included the following: (1) to determine the quantitative and qualitative toxicity of the human engineered anti-Ep-CAM antibody administered IV every week for six doses; (2) to assess the PK of the human engineered anti-Ep-CAM antibody when administered on this schedule; and (3) to assess the biodistribution of ¹³¹I-labeled human engineered anti-Ep-CAM antibody. Additional study objectives included the following: (1) to evaluate the ability of ¹³¹I-labeled human engineered anti-Ep-CAM antibody to localize different types of adenocarcinomas with high expression of antigen; and (2) to preliminarily document any antitumor activity of the human engineered anti-Ep-CAM antibody. This study was designed as an open-label, multi-dose, pilot study to evaluate the pharmacokinetics, safety, tolerability and biodistribution of six weekly 0.1 mg/kg doses of the human engineered anti-Ep-CAM antibody in subjects with advanced adenocarcinomas. Subjects were to receive the human engineered anti-Ep-CAM antibody as a single intravenous infusion over 1 hr to be repeated every week for six weeks. Subjects were to receive 1 mg of ¹³¹I-labeled human engineered anti-Ep-CAM antibody (10 mCi) as part of the first human engineered anti-Ep-CAM antibody dose. All subjects were to receive a minimum of six doses. At the end of Week 6, subjects were to be evaluated for retreatment and, if eligible for retreatment, subjects were to be dosed for another six weeks. No more than one subject was to be enrolled on any given day.

Subjects were eligible for inclusion in the study if they met all of the following criteria: (1) subject had documented histologic or cytologic diagnosis of adenocarcinomas of the lung, prostate, colon/rectum or pancreas that had been shown to express Ep-CAM; (2) subject had an advanced adenocarcinoma that was either refractory to standard therapies or for which therapies that may potentially be of major benefit did not exist; (3) subject's disease status was measurable and had at least one lesion of at least 2 cm, and subjects with prostate cancer were to have evaluable disease; (4) subject may have had prior radiation therapy if completed at least four weeks prior to study entry, the subject had recovered from the acute toxicities of that therapy, and measurable disease was in a non-irradiated area; (5) subject may have had prior chemotherapy, cytokine (such as IL-2, interferon, GM-CSF) therapy or immunotherapy if completed at least four weeks (six weeks for mitomycin and nitrosureas) prior to study entry and the subject had recovered from the acute toxicities of that therapy and Hormonal therapy for prostate cancer could be continued, but could not have been changed less than 4 weeks prior to study entry; (6) subject had a performance status of 0 to 2 on the Eastern Cooperative Oncology Group (ECOG) scale; (7) subject had an estimated life expectancy of at least 12 weeks; (8) subject was at least 18 years of age; (9) subject had adequate hematologic, renal, hepatic and pancreatic organ function (see Section 5.2.1 for specific criteria); (10) subject had signed the informed consent form prior to the performance of any study related procedure; (11) male and female subjects with reproductive potential had to use an approved contraceptive method (e.g., intrauterine device [IUD], birth control pills, or barrier device) during the study; and (12) female subjects with reproductive potential had to have a negative serum pregnancy test within seven days of study enrollment.

Subjects were to be excluded from the study for any of the following reasons: (1) subject had serious concomitant systemic disorders incompatible with the study such as NYHA class IIM, III or IV, or significant arrhythmias requiring therapy; (2) subject had used any investigational agent within 30 days of study entry; (3) subject was pregnant or lactating; (4) subject had undergone a bone marrow transplant; (5) subject was known to be HIV+, HBsAg+ or HCV+, or have any other recognized immunodeficiency disease; (6) subject had a history of severe allergic or anaphylactic reactions to monoclonal antibodies or antibody fragments; (7) subject had concurrent or prior malignancy, except for adequately-treated basal cell or squamous cell skin cancer, adequately-treated non-invasive carcinomas or other cancer from which the subject has been disease-free for at least five years; (8) subject had an active auto-immune disease requiring chronic treatment; (9) subject was using or had used immunosuppressive drugs such as cyclosporine, ACTH or corticosteroids within four weeks prior to enrollment; (10) subject had a brain metastases or a known history of brain metastases; (11) subject had a history of alcoholism or chronic pancreatitis or a family history of acute or chronic pancreatitis; or (12) subject had iodine sensitivity.

Six weekly doses of 0.1 mg/kg human engineered anti-Ep-CAM antibody were administered in this study, the first dose of which contained 1 mg of ¹³¹I-labeled human engineered anti-Ep-CAM antibody (10 mCi).

Safety variables for evaluation included: recording adverse clinical events; clinical laboratory assessments, including blood chemistries, hematology, urinalysis, hepatic enzymes, bilirubin, thyroid function tests, amylase, lipase, prothrombin time/international normalized ratio (PT/INR), and partial thromboplastin time (PTT); pretreatment and posttreatment physical examinations, including vital signs and electrocardiogram (ECG); human anti-human antibody (HAHA); pharmacokinetic blood sampling; concomitant medications; and toxicity rating. Efficacy variables for evaluation included: tumor measurements by CT-scan, chest X-ray, MRI, tumor markers or other appropriate methods to determine tumor response, and performance status using the ECOG scale. Whole body planar imaging was used to assess biodistribution and tumor localization with single-photon emission computerized tomography (SPECT) performed on suspected tumor lesions. These imaging scans took place at the end of infusion and 4, 24, 48, 96 and 168 hours post-infusion. Scans utilized a wide-field of view gamma camera with high energy (parallel hole collimator with 30% window centered at 364 KeV). Whole body probe counts were performed at the same time the imaging scans were taken.

Data were analyzed for safety, tolerability, preliminary efficacy, pharmacokinetics, and biodistribution, A detailed description of subject disposition was provided for all subjects. All adverse events (AEs), drug-related AEs, and serious adverse events (SAEs) were coded using the COSTART dictionary and presented for each subject. Laboratory values outside of the corresponding normal ranges were identified. All dosed subjects with data from a sufficient number of samples had the following pharmacokinetic parameters calculated from plasma concentrations of the human engineered anti-Ep-CAM antibody: serum concentration, peak plasma drug concentration (Cₘₐₓ), area under the serum concentration versus the time curve (AUC), systemic clearance (CL), central volume of drug distribution (Vc) and half-life (t_{1/2}). Levels of plasma the human engineered anti-Ep-CAM antibody in subjects for whom levels were available were described over time. To assess the preliminary clinical efficacy of the human engineered anti-Ep-CAM antibody, tabulations of mortality, tumor markers and tumor response were presented for each subject.

Only three subjects were enrolled in this study. The first subject enrolled had pancreatic cancer and was taken off the study due to progressive disease after his second dose. The second subject enrolled had prostate cancer, received six doses and was taken off study due to progressive disease. The third subject enrolled had colorectal cancer and received 24 doses, though the subject met the criteria for progressive disease at Week 20.

The following results were obtained in this small, Phase I study: (1) intravenous weekly administration of the human engineered anti-Ep-CAM antibody at 0.1 mg/kg for treating subjects suffering from advanced adenocarcinomas was safe and well-tolerated; (2) the average circulating half-life of the human engineered anti-Ep-CAM antibody at 0.1 mg/kg was 26 hours and this dose level ensures a detectable drug level for one week, with no accumulation observed; (3) no objective tumor responses were seen but one subject had stable disease at 12 weeks (the subject's CEA levels were low and remained low while on study); (4) one subject showed tumor localization to two liver metastases in the liver; (5) tissue retention of the human engineered anti-Ep-CAM antibody was longer than plasma half-life; and (6) no HAHA response could be detected.

C. Study 3

This third study was an open-label, multi-dose, Phase I, dose-escalating study of a subcutaneously administered human engineered anti-Ep-CAM antibody in subjects with advanced adenocarcinomas.

Study objectives included the following: (1) to determine the MTD of the human engineered anti-Ep-CAM antibody administered subcutaneously; (2) to determine the average bioavailability of subcutaneously administered human engineered anti-Ep-CAM antibody; (3) to evaluate the safety, immunogenicity, and tolerability of weekly doses of the human engineered anti-Ep-CAM antibody administered subcutaneously; (4) to assess the pharmacokinetics of the human engineered anti-Ep-CAM antibody when administered on this schedule and to relate pharmacokinetics to drug effects (toxicity and activity), wherever possible; and (5) to preliminarily document any antitumor activity of the human engineered anti-Ep-CAM antibody.

This study was designed as an open-label, multi-dose, Phase I, dose-escalating study to evaluate the average bioavailability, safety, tolerability, immunogenicity, and pharmacokinetics of the human engineered anti-Ep-CAM antibody administered subcutaneously in subjects with advanced adenocarcinomas. At least four dose groups were planned for this study and a minimum of three subjects were planned to be enrolled per dose group. Subjects were assigned sequentially to the same dose group until all subjects for that dose group have been enrolled. Subjects could be enrolled in the next higher dose group once at least three subjects received six doses and were followed for a week after the sixth dose (Day 43), provided no dose limiting toxicity (DLT) was observed. Subjects received the human engineered anti-Ep-CAM antibody weekly for six weeks. All subjects were dosed subcutaneously. All subjects entered into the study were closely and continually monitored for safety, tolerability, immunogenicity, pharmacokinetics and tumor response. Subject safety was monitored by adverse event reporting and clinical assessment based on physical examination including vital signs and laboratory tests (including blood chemistries, hepatic enzymes, bilirubin, amylase, lipase, hematology, PT/INR, PTT and urinalysis). Plasma levels of study medication were assessed by serial monitoring of blood samples. Subjects were followed for tumor response by CT-scan, chest X-ray, MRI, tumor markers or other appropriate methods. Subjects were screened and monitored for the development of human-anti-human antibodies (HAHA).

The study population included subjects with adenocarcinomas of the ovary, breast, lung, prostate, colon or rectum that were either refractory to standard therapies or for which therapies that could potentially be of major benefit did not exist. All subjects received subcutaneously administered human engineered anti-Ep-CAM antibody on a weekly basis for 6 weeks. Provided subjects met certain criteria, dosing could continue beyond 6 weeks. Since this was an open-label uncontrolled study, the identity of the test article was known to both the investigator and the subject. Four dose levels were evaluated by adding cohorts of three subjects at each of the following dose levels: 0.1 mg/kg, 0.3 mg/kg, 0.6 mg/kg, and 0.8 mg/kg. Monitoring subjects for safety was the primary objective of this study. Multiple subject assessments of vital signs, physical exams, and clinical tests were utilized. Concomitant medications and adverse events were evaluated and tracked.

The results of immunogenicity studies from these clinical studies were described in Example 12.

### EXAMPLE 12

### Immunogenicity Studies in Humans

A human engineered anti-Ep-CAM antibody produced by Clone 146 as described in Example 2B above was administered to humans in three Phase I clinical trials as described in Example 11 and tested for immunogenicity as follows.

A. Study 1

In a first Phase I study, subjects with advanced adenocarcinoma were assigned to one of four dose groups and treated with a single intravenous dose of a human engineered anti-Ep-CAM antibody every 21 days for up to four doses at either 0.03, 0.1, 0.03 or 1 mg/kg. Plasma samples for measurement of antibody response to the human engineered anti-Ep-CAM antibody were collected pre-dose, and when available at study days 22, 43, 64, and 84 (every 3 weeks).

For the measurement of human antibodies to the human engineered anti-Ep-CAM antibody in serum or plasma, a double antigen sandwich ELISA was used as follows. The human engineered anti-Ep-CAM antibody was diluted to 0.10 mg/mL in phosphate buffered saline, pH 7.2 (PBS). Fifty mL of this solution were added to individual wells of Immulon 2 microtiter plates (Dynatech Laboratories, Chantilly, Va.) and incubated overnight at 2-8° C. The antibody solution was removed and 150 mL of 1% bovine serum albumin (BSA) in PBS containing 0.05% Tween 20 (PBS/T) was added to all wells. Microtiter plates were then incubated for 1 hour at room temperature. After blocking, the wells of each plate were washed three times with 300 mL of wash buffer (PBS containing 0.05% Tween 20). Standards, samples and controls were diluted in triplicate with 1% BSA-PBS/T in separate 96-well plates. Affinity purified goat anti-human IgG antibody standard (Zymed Laboratories, South San Francisco, Calif.) was prepared as serial two-fold dilutions from 10,000 to 1.2 ng/mL. Each replicate and dilution of the standards, samples and controls (50 mL) was transferred to the blocked microtiter plates and incubated for 1 hour at 37° C. After the primary incubation, the wells were washed 3 times with wash buffer. Biotin-labeled human engineered anti-Ep-CAM antibody derivatized via carbohydrate or amino groups were diluted to approximately 250 ng/mL each in 1.0% BSA-PBS/T. Fifty mL of biotin-labeled human engineered anti-Ep-CAM antibody were added to all wells. The plates were then incubated for 1 hour at 37° C.

Subsequently, all wells were washed 3 times with wash buffer. Alkaline phosphatase-labeled streptavidin (Zymed Laboratories, South San Francisco, Calif.) was diluted 1/2000 in 1% BSA-PBS/T and 50 mL was added to all wells. After incubation for 15 minutes at 37° C., all wells were washed 3 times with wash buffer and 3 times with deionized water and the substrate p-nitrophenylphosphate (1 mg/mL in 10% diethanolamine buffer, pH 9.8) was added in a volume of 50 mL to all wells. Color development was allowed to proceed for 1 hour at room temperature, after which 50 mL of 1 N NaOH was added to stop the reaction. The absorbance at 405 nm was determined for all wells using a Vmax Plate Reader (Molecular Devices, Menlo Park, Calif.).

The mean absorbance at 405 nm (A405) was calculated for all standards, samples and controls (in triplicate). A standard curve was generated as a 4-parameter fit of the A405 versus ng/mL of goat anti-human IgG antibody standard. Test samples were considered to be positive for human antibodies to the human engineered anti-Ep-CAM antibody if the average A405 was greater than A405 for the reagent control+3 standard errors of the mean (SEM). Positive samples were quantified by interpolation from the standard curve and are expressed in ng of goat anti-human IgG equivalents/mL (ng equivalents/mL). The detection limit for this assay was approximately 10 ng/mL.

Competition experiments were also conducted with the human engineered anti-Ep-CAM antibody, human IgG1, and a control human engineered antibody not directed to Ep-CAM but of the same isotype (he4A2). For each positive sample, a competition experiment was conducted using the standard assay format described above with the following modifications: 10 mg/mL (twenty fold excess) of unlabeled human engineered anti-Ep-CAM antibody, human IgG, or the human engineered isotype control he4A2 was pre-mixed with the biotin human engineered anti-Ep-CAM antibody detector. Goat anti-human IgG equivalents were calculated for each sample and competitor tested. Evidence of competition was demonstrated by at least a 25% reduction in the measured goat anti-human IgG equivalents for any of the competitors tested when compared to the absence of competitor.

Antibody response to the human engineered anti-Ep-CAM antibody was evaluable in 17 of 22 patients in this first open-label phase I clinical trial. An antibody response was detected in 2 of 17 subjects (11.8%) for at least one post treatment sample. When segregated by dose, the response rate was 0 of 4 in the 0.03 mg/kg dose group, 0 of 3 for the 0.1 mg/kg dose group, and 2 of 10 for the 0.3 mg/kg dose group. The maximal response was 188 ng equivalents/mL of goat anti-human IgG standard (range 11-188 ng equivalents/mL). No antibody response was detected in the pretreatment samples. One subject had an antibody response after the second dose which increased after a subsequent dose, while the second subject developed an antibody response after the third dose.

The specificity of the antibody response was determined from competition experiments performed to ascertain if the positive signals generated could be inhibited with the human engineered anti-Ep-CAM antibody, the human engineered isotype control he4A2, or human IgG. For each positive sample, the assay was repeated under standard conditions except that a 20 fold excess of human engineered anti-Ep-CAM antibody, he4A2, or polyclonal human IgG was added to the biotin-human engineered anti-Ep-CAM antibody detector. As expected, the presence of unlabeled human engineered anti-Ep-CAM antibody completely inhibited the signal in the assay. In the presence of he4A2, assay signal was reduced by 22-34%. Human IgG did not significantly reduce (inhibition≧25%) assay signal. These competition experiments suggest that the antibody response is directed towards the variable region since no inhibition was seen with the human IgG. Inhibition with he4A2 indicates partial homology with the human engineered anti-Ep-CAM variable region. Results of sequence analysis of the variable region of he4A2 and the human engineered anti-Ep-CAM heavy and light chains indicate a 50-60% homology in the variable region of these two antibodies.

In summary, antibody response to a human engineered anti-Ep-CAM antibody was assesssed in 17 of 22 patients enrolled in an open-label, multi-dose, phase I study of subjects with advanced adenocarcinomas. A total of 2 out of 17 evaluable subjects (11.8%) had a detectable antibody response for at least 1 sample. One subject had an antibody response after the second dose which increased after a subsequent dose, while the second subject developed an antibody response after the third dose. Results of the competition experiments suggest that the antibody response is directed towards the variable region since no inhibition was seen with the polyclonal human IgG.

B. Study 2

In a second Plase I study, subjects with advanced adenocarcinomas were treated with 0.1 mg/kg of a human engineered anti-Ep-CAM antibody as a single 1 hour intravenous infusion every week for six weeks. At the end of week six, subjects were evaluated for continued dosing. Plasma samples for measurement of antibody response to the human engineered anti-Ep-CAM antibody were collected pre-dose, and when available at study days 22 and 43. If the patient was eligible for continued dosing, samples were collected prior to each dose and at the end of the study. A double antigen sandwich ELISA was used as described in Part A of this Example for the measurement of human antibodies to the human engineered anti-Ep-CAM antibody in serum or plasma. Competition experiments were also conducted with the human engineered anti-Ep-CAM antibody, human IgG and the human engineered control isotype antibody he4A2 as described in Part A of this Example. Thus, the antibody response to the human engineered anti-Ep-CAM antibody was assessed in patients enrolled in this second open label, multiple-dose study of subjects with advanced adenocarcinomas.

None of the patients in this study had a detectable antibody response. One patient had only a pretreatment sample taken. A second patient had only 1 post treatment sample, that was collected prior to receiving a fifth 1-hour infusion. A third patient remained on study for an extended period and received 24 infusions of antibody.

C. Study 3

In a third Phase I study, patients with advanced adenocarcinoma were treated with weekly subcutaneous doses of a human engineered anti-Ep-CAM antibody, for example, at 0.1 mg/kg, 0.3 mg/kg, 0.6 mg/kg, or 0.8 mg/kg for six weeks. Plasma samples for measurement of antibody response to the human engineered anti-Ep-CAM antibody were collected pre-dose, and when available every three weeks or until end of treatment.

A double antigen sandwich ELISA was used as described in Part A of this Example for the measurement of human antibodies to the human engineered anti-Ep-CAM antibody in serum or plasma. Competition experiments were also conducted with the human engineered anti-Ep-CAM antibody, and human engineered control isotype antibody he4A2 as described in Part A of this Example. Thus, the antibody response to the human engineered anti-Ep-CAM antibody was evaluable in 13 of 14 patients in this third open-label Phase I clinical trial.

An antibody response was detected in 3 of the 13 patients (23%) for at least one post treatment sample. No antibody response was detected in the pretreatment samples. The maximal antibody response was 85.8 ng equivalents/mL of goat anti-human IgG standard. When detected, antibody response occurred after study day 22.

In order to determine the specificity of the antibody response, competition experiments were preformed to ascertain if the positive signals generated could be inhibited with the human engineered anti-Ep-CAM antibody, or the human engineered control isotype antibody he4A2. For each positive sample, the assay was repeated under standard condition except that a 20-fold excess of human engineered anti-Ep-CAM, or he4A2 was added to the biotin human engineered anti-Ep-CAM antibody detector. As expected, the presence of unlabeled human engineered anti-Ep-CAM antibody completely inhibited the signal in the assay. In the presence of he4A2, assay signal was reduced by 32-37%. As stated above, results of sequence analysis of the variable region of he4A2 and the human engineered anti-Ep-CAM heavy and light chains indicate 50-60% homology in the variable region of these two antibodies. Inhibition with he4A2 indicates partial homology with the variable region. The competition experiments suggest that the antibody response is directed primarily towards the variable region of ING-1.

In summary, antibody response to a human engineered anti-Ep-CAM antibody was assessed in 13 of 14 patients enrolled in an open label, multi-dose, phase I study of patients with advanced adenocarcinomas. Three of the thirteen (23%) patients enrolled had a post treatment sample with a detectable HAHA response. Results of the competition experiments suggest that the antibody response is directed towards to variable region.

Although human engineering of an anti-Ep-CAM antibody according to the method of Studnicka as described herein was expected to reduce the immunogenicity of the antibody, the results from these human studies indicate that the human engineered anti-Ep-CAM antibody is surprisingly non-immunogenic in humans. In particular, the results from these three Phase I human studies show that in the majority of subjects evaluated, no human anti-human antibody (HAHA) response was detected. Even where antibody was detected, it was at low levels (e.g., ng/mL) and was directed to the variable region (e.g., anti-idiotypic). These results may be considered even more surprising since they were obtained with a double antigen sandwich ELISA assay that was optimized as described herein to be highly sensitive (e.g., detection limit of about 10 ng/mL) and under conditions where a low threshold for positivity was set relying on a statistical difference with the negative control (e.g., sample considered positive if the OD measured was statistically significantly different from the OD of the negative control with no antibody present). Thus, the frequency and magnitude of any response detected with this optimized assay are magnified to maximize the detection of any level, even very low levels, of antibody. The data reflecting the percentage of subjects whose assay results were considered positive for antibody to the administered antibody are highly dependent on the sensitivity and specificity of the assay. The observed frequency of antibody positivity may additionally be influenced by other factors, including subject population (e.g., underlying disease), concomitant medications and sample handling. For these reasons and others, comparison of the incidence of antibodies to the human engineered anti-Ep-CAM antibody to other antibody products may be misleading. Nevertheless, the results from these studies indicate that the human engineered anti-Ep-CAM antibody is no more immunogenic in humans than are antibodies humanized by other methods, or therapeutic antibodies developed from transgenic mice or phage display.

All patents, patent applications, literature publications and test methods cited herein are hereby incorporated by reference. The reader's attention is directed to all papers and documents which are filed concurrently with or previous to this specification in connection with this application and which are open to public inspection with this specification, and the contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is only one example of a generic series of equivalent or similar features.

Although the present invention has been described in considerable detail with reference to certain preferred versions thereof, other versions are possible. Many variations of the present invention will suggest themselves to those skilled in the art in light of the above detailed disclosure. All such modifications are within the full intended scope of the appended claims. Therefore, the spirit and scope of the appended claims should not be limited to the description of the preferred embodiments contained herein.

1. A human engineered anti-Ep-CAM antibody which binds specifically to human Ep-CAM comprising a heavy chain variable region comprising the amino acid sequence of SEQ ID NO: 19 or SEQ ID NO: 21.

2. A human engineered anti-Ep-CAM antibody which binds specifically to human Ep-CAM comprising a light chain variable region comprising the amino acid sequence of SEQ ID NO: 6, SEQ ID NO: 8, SEQ ID NO: 35, SEQ ID NO: 37, SEQ ID NO: 39, SEQ ID NO: 41, SEQ ID NO: 43, or SEQ ID NO: 45.

3. A human engineered anti-Ep-CAM antibody which specifically binds to human Ep-CAM comprising a heavy chain variable region comprising the amino acid sequence of SEQ ID NO: 19 and a light chain variable region comprising the amino acid sequence of SEQ ID NO: 6.

4. A human engineered anti-Ep-CAM antibody of any one of claims 1 to 3 which is a full length antibody.

5. A human engineered anti-Ep-CAM antibody of any one of claims 1 to 3 which is a human IgG.

6. A human engineered anti-Ep-CAM antibody of any one of claims 1 to 3 which is an antibody fragment.

7. A human engineered anti-Ep-CAM antibody of claim 6 wherein the antibody fragment is a F(ab)₂, Fab, Fv or ScFv.

8. A labeled antibody comprising the human engineered anti-Ep-CAM antibody of any one of claims 1 to 3 bound to a detectable label.

9. An immobilized antibody comprising the human engineered anti-Ep-CAM antibody of any one of claims 1 to 3 bound to a solid phase.

10. A conjugate comprising the human engineered anti-Ep-CAM antibody of any one of claims 1 to 3 bound to a cytotoxic or non-cytotoxic agent.

11. A method for determining the presence of Ep-CAM protein comprising exposing a sample suspected of containing the Ep-CAM protein to the human engineered anti-Ep-CAM antibody of any one of claims 1 to 3 and determining binding of the antibody to the sample.

12. A kit comprising the human engineered anti-Ep-CAM antibody of any one of claims 1 to 3 and instructions for using the human engineered anti-Ep-CAM antibody to detect the Ep-CAM protein.

13. Isolated nucleic acid sequence encoding the Ep-CAM antibody of claim 1.

14. Isolated nucleic acid sequence encoding the Ep-CAM antibody of claim 2.

15. Isolated nucleic acid sequence encoding the Ep-CAM antibody of claim 3.

16. A vector comprising the nucleic acid sequence of claim 13.

17. A vector comprising the nucleic acid sequence of claim 14.

18. A vector comprising the nucleic acid sequence of claim 15.

19. A host cell comprising the nucleic acid sequence of claim 13.

20. A host cell comprising the nucleic acid sequence of claim 14.

21. A host cell comprising the nucleic acid sequence of claim 15.

22. A process of producing human engineered anti-Ep-CAM antibody comprising culturing a host cell comprising the nucleic acid sequence of claim 13 so that the nucleic acid sequence is expressed.

23. A process of producing human engineered anti-Ep-CAM antibody comprising culturing a host cell comprising the nucleic acid sequence of claim 14 so that the nucleic acid sequence is expressed.

24. A process of producing human engineered anti-Ep-CAM antibody comprising culturing a host cell comprising the nucleic acid sequence of claim 15 so that the nucleic acid sequence is expressed.

25. The process of claim 22 further comprising recovering the human engineered anti-Ep-CAM antibody from the host cell culture.

26. The process of claim 23 further comprising recovering the human engineered anti-Ep-CAM antibody from the host cell culture.

27. The process of claim 24 further comprising recovering the human engineered anti-Ep-CAM antibody from the host cell culture.

28. A composition comprising the human engineered anti-Ep-CAM antibody of claim 1 and a pharmaceutically acceptable carrier or diluent.

29. A composition comprising the human engineered anti-Ep-CAM antibody of claim 2 and a pharmaceutically acceptable carrier or diluent.

30. A composition comprising the human engineered anti-Ep-CAM antibody of claim 3 and a pharmaceutically acceptable carrier or diluent.

31. A method for treating a mammal suffering from an Ep-CAM mediated disease, disorder or condition comprising administering a pharmaceutically effective amount of the human engineered anti-Ep-CAM antibody of claim 1 to the mammal.

32. A method for treating a mammal suffering from an Ep-CAM mediated disease, disorder or condition comprising administering a pharmaceutically effective amount of the human engineered anti-Ep-CAM antibody of claim 2 to the mammal.

33. A method for treating a mammal suffering from an Ep-CAM mediated disease, disorder or condition comprising administering a pharmaceutically effective amount of the human engineered anti-Ep-CAM antibody of claim 3 to the mammal.

34. The method of claim 31 further comprising administering a chemotherapeutic agent before, after or simultaneously with the human engineered anti-Ep-CAM antibody.

35. The method of claim 32 further comprising administering a chemotherapeutic agent before, after or simultaneously with the human engineered anti-Ep-CAM antibody.

36. The method of claim 33 further comprising administering a chemotherapeutic agent before, after or simultaneously with the human engineered anti-Ep-CAM antibody.

37. A method for determining the presence of a human antibody made by a subject in response to administration to the subject of the human engineered anti-Ep-CAM antibody of any one of claims 1, 2 or 3 comprising exposing a sample suspected of containing the human antibody to the human engineered anti-Ep-CAM antibody and determining the binding of the human antibody to the sample.

38. The method of claim 37 wherein the sample is blood, serum or plasma.


SEQUENCE LISTING (59 sequences)

SEQ ID NO: 1 - DNA, 720 bp, Homo Sapiens
  misc_feature: Mouse Human Chimeric Light Chain DNA and Protein
atgaggttctctgctcagcttctggggctgcttgtgctctggatccctggatccactgcagatattgtgatgacgcaggctgcattctccaatccagtcactcttggaacatcaggttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttcctcggacgttcggtggaggcaccaagcttgagatgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 2 - protein, 239 aa, Homo Sapiens
MRFSAQLLGLLVLWIPGSTADIVMTQAAFSNPVTLGTSGSISCRSSKSLLHSNGITYLYWYLQKPGQSPQLLIYQMSNLASGVPDRFSSSGSGTDFTLRISRVEAEDVGVYYCAQNLELPRTFGGGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 1398 bp, Homo Sapiens
  misc_feature: Mouse-Human chimeric Heavy Chain DNA and Protein Sequence
atggcttgggtgtccaccttgctattcctgatggcagctgcccaaagtgcccaagcacagatccagttggtgcagtctggacctgagctgaagaagcctggagagacagtcaagatctcctgcaaggcttctggatataccttcacaaaatatggaatgaactgggtgaagcaggctccaggaaagggtttaaagtggatgggctggataaacacctacactgaagagccaacatatggtgatgacttcaagggacggtttgccttctctttggaaacctctgccagcactgccaatttgcagatcaacaacctcaaaagtgaggacacggctacatatttctgtgcaagatttggctctgctgtggactactggggtcaaggaacctcggtcaccgtctcctcagccagcacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - protein, 465 aa, Homo Sapiens
MAWVSTLLFLMAAAQSAQAQIQLVQSGPELKKPGETVKISCKASGYTFTKYGMNWVKQAPGKGLKWMGWINTYTEEPTYGDDFKGRFAFSLETSASTANLQINNLKSEDTATYFCARFGSAVDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 720 bp, Homo Sapiens
  misc_feature: Low Risk Human Engineered ING-1 Light Chain (LC)
atgaggttctctgctcagcttctggggctgcttgtgctctggatccctggatccactgcagacatcgtgatgacccagtctgcactctccaatccagtcactctgggagagtcaggttccatctcctgccggtctagtaagagtctcctacatagtaatggcatcacttatttgtattggtatctgcagaaaccagggcagtctcctcagctgctcatctatcagatgtctaacagagcctcaggggtcccagacaggttcagtagcagtggatctgggacagatttcactctcaagatcagcagagtggaggctgaagatgtgggagtttattactgtgctcagaacctagagcttccgcggacgttcggtcagggcaccaagcttgagatgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 6 - protein, 239 aa, Homo Sapiens
MRFSAQLLGLLVLWIPGSTADIVMTQSALSNPVTLGESGSISCRSSKSLLHSNGITYLYWYLQKPGQSPQLLIYQMSNRASGVPDRFSSSGSGTDFTLKISRVEAEDVGVYYCAQNLELPRTFGQGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 720 bp, Homo Sapiens
  misc_feature: Low + Moderate Risk Human Engineered ING-1 Light Chain (LC)
atgaggttctctgctcagcttctggggctgcttgtgctctggatccctggatccactgcagacatcgtgatgacccagtctccactctccctgccagtcactcctggagagccgggttccatctcctgccggtctagtaagagtctcctacatagtaatggcatcacttatttgtattggtatctgcagaaaccagggcagtctcctcagctgctcatctatcagatgtctaacagagcctcaggggtcccagacaggttcagtagcagtggatctgggacagatttcactctcaagatcagcagagtggaggctgaagatgtgggagtttattactgtgctcagaacctagagcttccacggacgttcggtcagggcaccaagcttgagatgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 8 - protein, 239 aa, Homo Sapiens
MRFSAQLLGLLVLWIPGSTADIVMTQSPLSLPVTPGEPGSISCRSSKSLLHSNGITYLYWYLQKPGQSPQLLIYQMSNRASGVPDRFSSSGSGTDFTLKISRVEAEDVGVYYCAQNLELPRTFGQGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 88 bp, Homo Sapiens
  misc_feature: KL1 V Region Oligos Human Engineered ING-1 Light Chain (Kappa low)
tgtcgacaccatgaggttctctgctcagcttctggggctgcttgtgctctggatccctggatccactgcagacatcgtgatgacccag

SEQ ID NO: 10 - DNA, 85 bp, Homo Sapiens
  misc_feature: KL2 V Region Oligos Human Engineered ING-1 Light Chain (Kappa low)
actcttactagaccggcaggagatggaacctgactctcccagagtgactggattggagagtgcagactgggtcatcacgatgtct

SEQ ID NO: 11 - DNA, 88 bp, Homo Sapiens
  misc_feature: KL3 V Region Oligos Human Engineered ING-1 Light Chain (Kappa low)
ctgccggtctagtaagagtctcctacatagtaatggcatcacttatttgtattggtatctgcagaaaccagggcagtctcctcagctg

SEQ ID NO: 12 - DNA, 86 bp, Homo Sapiens
  misc_feature: KL4 V Region Oligos Human Engineered ING-1 Light Chain (Kappa low)
tgtcccagatccactgctactgaacctgtctgggacccctgaggctctgttagacatctgatagatgagcagctgaggagactgcc

SEQ ID NO: 13 - DNA, 77 bp, Homo Sapiens
  misc_feature: KL5 V Region Oligos Human Engineered ING-1 Light Chain (Kappa low)
agcagtggatctgggacagatttcactctcaagatcagcagagtggaggctgaagatgtgggagtttattactgtgc

SEQ ID NO: 14 - DNA, 75 bp, Homo Sapiens
  misc_feature: KL6 V Region Oligos Human Engineered ING-1 Light Chain (Kappa low)
tttgatttcaagcttggtgccctgaccgaacgtccgtggaagctctaggttctgagcacagtaataaactcccac

SEQ ID NO: 15 - DNA, 22 bp, Homo Sapiens
  misc_feature: Low Risk Primers Forward Primer KF ING-1 Light Chain Oligos
ttatgtcgacaccatgaggttc

SEQ ID NO: 16 - DNA, 21 bp, Homo Sapiens
  misc_feature: Low risk Primers Reverse Primer  KR ING-1 Light Chain Oligos
tttgatttcaagcttggtgcc

SEQ ID NO: 17 - DNA, 85 bp, Homo Sapiens
  misc_feature: Moderate Risk Primer KM2 V Region Oligos Human Engineered ING-1 Light Chain Oligos (Kappa Moderate)
actcttactagaccggcaggagatggaacccggctctccaggagtgactggcagggagagtggagactgggtcatcacgatgtct

SEQ ID NO: 18 - DNA, 1398 bp, Homo Sapiens
  misc_feature: Low Risk Human Engineered ING-1 Heavy Chain (HC)
atggcttgggtgtccaccttgctattcctgatggcagctgcccaaagtgcccaagcacagatccagttggtgcagtctggacctgaggtgaagaagcctggagagtccgtcaagatctcctgcaaggcttctggatataccttcacaaaatatggaatgaactgggtgaagcaggctccaggacagggtttaaagtggatgggctggataaacacctacactgaagagccaacatatggtgatgacttcaagggacggtttaccttcaccttggacacctctactagcactgcctatttggaaatctcttctctccggagtgaggacacggctacatatttctgtgcaagatttggctctgctgtggactactggggtcaaggaaccttggtcaccgtctcctcagccagcacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 19 - protein, 465 aa, Homo Sapiens
MAWVSTLLFLMAAAQSAQAQIQLVQSGPEVKKPGESVKISCKASGYTFTKYGMNWVKQAPGQGLKWMGWINTYTEEPTYGDDFKGRFTFTLDTSTSTAYLEISSLRSEDTATYFCARFGSAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 1398 bp, Homo Sapiens
  misc_feature: Low + Moderate Risk Human Engineered ING-1 Heavy Chain (HC)
atggcttgggtgtccaccttgctattcctgatggcagctgcccaaagtgcccaagcacagatccagttggtgcagtctggagctgaggtgaagaagcctggagagtcagtcaagatctcctgcaaggcttctggatataccttcacaaaatatggaatgaactgggtgcgacaggctccaggacaaggtttagagtggatgggctggataaacacctacactgaagagccaacatatggtcagaagttccagggacggtttaccttcaccttggacacctctactagcactgcctatttggaaatctcttcgctccggagtgaggacacggctgtgtatttctgtgcaagatttggctctgctgtggactactggggtcaaggaaccttggtcaccgtctcctcagccagcacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 21 - protein, 465 aa, Homo Sapiens
MAWVSTLLFLMAAAQSAQAQIQLVQSGAEVKKPGESVKISCKASGYTFTKYGMNWVRQAPGQGLEWMGWINTYTEEPTYGQKFQGRFTFTLDTSTSTAYLEISSLRSEDTAVYFCARFGSAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 91 bp, HomoSapiens
  misc_feature: GL1 V Region Oligos Human Engineered ING-1 Heavy Chain Oligos (gamma low)
tgtcgacaccatggcttgggtgtccaccttgctattcctgatggcagctgcccaaagtgcccaagcacagatccagttggtgcagtctgga

SEQ ID NO: 23 - DNA, 90 bp, HomoSapiens
  misc_feature: GL2 V Region Oligos Human Engineered ING-1 Heavy Chain Oligos (gamma low)
atattttgtgaaggtatatccagaagccttgcaggagatcttgacggactctccaggcttcttcacctcaggtccagactgcaccaactg

SEQ ID NO: 24 - DNA, 91 bp, HomoSapiens
  misc_feature: GL3 V Region Oligos Human Engineered ING-1 Heavy Chain Oligos (gamma low)
tggatataccttcacaaaatatggaatgaactgggtgaagcaggctccaggacagggtttaaagtggatgggctggataaacacctacact

SEQ ID NO: 25 - DNA, 90 bp, HomoSapiens
  misc_feature: GL4 V Region Oligos Human Engineered ING-1 Heavy Chain Oligos (gamma low)
cagtgctagtagaggtgtccaaggtgaaggtaaaccgtcccttgaagtcatcaccatatgttggctcttcagtgtaggtgtttatccagc

SEQ ID NO: 26 - DNA, 90 bp, HomoSapiens
  misc_feature: GL5 V Region Oligos  Human Engineered ING-1 Heavy Chain Oligos (gamma low)
gacacctctactagcactgcctatttggaaatctcttctctccggagtgaggacacggctacatatttctgtgcaagatttggctctgct

SEQ ID NO: 27 - DNA, 85 bp, HomoSapiens
  misc_feature: GL6 V Region Oligos Human Engineered ING-1 Heavy Chain Oligos (gamma low)
gaccgatgggccctttgtgctggctgaggagacggtgaccaaggttccttgaccccagtagtccacagcagagccaaatcttgca

SEQ ID NO: 28 - DNA, 22 bp, HomoSapiens
  misc_feature: Human Engineered ING-1 Heavy Chain Oligos-Low Risk Primers Forward primerGF
ttatgtcgacaccatggcttgg

SEQ ID NO: 29 - DNA, 17 bp, HomoSapiens
  misc_feature: Human Engineered ING-1 Heavy Chain Oligos Low Risk Primers-Reverse Primer GR
gaccgatgggccctttg

SEQ ID NO: 30 - DNA, 90 bp, HomoSapiens
  misc_feature: GM2 V Region Oligos Human Engineered ING-1 Heavy Chain Oligos-Low + Moderate Risk Primers
atattttgtgaaggtatatccagaagccttgcaggagatcttgactgactctccaggcttcttcacctcagctccagactgcaccaactg

SEQ ID NO: 31 - DNA, 91 bp, HomoSapiens
  misc_feature: GM3 V Region Oligos Human Engineered ING-1 Heavy Chain Oligos-Low + Moderate Risk Primers
tggatataccttcacaaaatatggaatgaactgggtgcgacaggctccaggacaaggtttagagtggatgggctggataaacacctacact

SEQ ID NO: 32 - DNA, 90 bp, HomoSapiens
  misc_feature: GM4 V Region Oligos Human Engineered ING-1 Heavy Chain Oligos-Low + Moderate Risk Primers
cagtgctagtagaggtgtccaaggtgaaggtaaaccgtccctggaacttctgaccatatgttggctcttcagtgtaggtgtttatccagc

SEQ ID NO: 33 - DNA, 90 bp, HomoSapiens
  misc_feature: GM5 V Region Oligos Human Engineered ING-1 Heavy Chain Oligos-Low + Moderate Risk Primers
gacacctctactagcactgcctatttggaaatctcttcgctccggagtgaggacacggctgtgtatttctgtgcaagatttggctctgct

SEQ ID NO: 34 - DNA, 720 bp, Homo sapiens
  misc_feature: P1=P Human Engineered (low risk) ING1 light Chain with one moderate risk proline change; proline at position 8 (P1)
atgaggttctctgctcagcttctggggctgcttgtgctctggatccctggatccactgcagacatcgtgatgacccagtctccactctccaatccagtcactctgggagagtcaggttccatctcctgccggtctagtaagagtctcctacatagtaatggcatcacttatttgtattggtatctgcagaaaccagggcagtctcctcagctgctcatctatcagatgtctaacagagcctcaggggtcccagacaggttcagtagcagtggatctgggacagatttcactctcaagatcagcagagtggaggctgaagatgtgggagtttattactgtgctcagaacctagagcttccgcggacgttcggtcagggcaccaagcttgagatgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 35 - protein, 239 aa, Homo sapiens
MRFSAQLLGLLVLWIPGSTADIVMTQSPLSNPVTLGESGSISCRSSKSLLHSNGITYLYWYLQKPGQSPQLLIYQMSNRASGVPDRFSSSGSGTDFTLKISRVEAEDVGVYYCAQNLELPRTFGQGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - DNA, 720 bp, Homo sapiens
  misc_feature: P2=P Human Engineered (low risk) ING1 light Chain with one moderate risk proline change; proline at position 15 (P2)
atgaggttctctgctcagcttctggggctgcttgtgctctggatccctggatccactgcagacatcgtgatgacccagtctgcactctccaatccagtcactcctggagagtcaggttccatctcctgccggtctagtaagagtctcctacatagtaatggcatcacttatttgtattggtatctgcagaaaccagggcagtctcctcagctgctcatctatcagatgtctaacagagcctcaggggtcccagacaggttcagtagcagtggatctgggacagatttcactctcaagatcagcagagtggaggctgaagatgtgggagtttattactgtgctcagaacctagagcttccgcggacgttcggtcagggcaccaagcttgagatgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 37 - protein, 239 aa, Homo sapiens
MRFSAQLLGLLVLWIPGSTADIVMTQSALSNPVTPGESGSISCRSSKSLLHSNGITYLYWYLQKPGQSPQLLIYQMSNRASGVPDRFSSSGSGTDFTLKISRVEAEDVGVYYCAQNLELPRTFGQGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 720 bp, Homo sapiens
  misc_feature: P3=P Human Engineered (low risk) ING1 light Chain with one moderate risk proline change; proline at position 18 (P3)
atgaggttctctgctcagcttctggggctgcttgtgctctggatccctggatccactgcagacatcgtgatgacccagtctgcactctccaatccagtcactctgggagagccgggttccatctcctgccggtctagtaagagtctcctacatagtaatggcatcacttatttgtattggtatctgcagaaaccagggcagtctcctcagctgctcatctatcagatgtctaacagagcctcaggggtcccagacaggttcagtagcagtggatctgggacagatttcactctcaagatcagcagagtggaggctgaagatgtgggagtttattactgtgctcagaacctagagcttccgcggacgttcggtcagggcaccaagcttgagatgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 39 - protein, 239 aa, Homo sapiens
MRFSAQLLGLLVLWIPGSTADIVMTQSALSNPVTLGEPGSISCRSSKSLLHSNGITYLYWYLQKPGQSPQLLIYQMSNRASGVPDRFSSSGSGTDFTLKISRVEAEDVGVYYCAQNLELPRTFGQGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 720 bp, Homo sapiens
  misc_feature: P1P2=Human Engineered (low risk) ING1 light Chain with one moderate risk proline change; proline at position 8 (P1) 15(P2)
atgaggttctctgctcagcttctggggctgcttgtgctctggatccctggatccactgcagacatcgtgatgacccagtctccactctccaatccagtcactcctggagagtcaggttccatctcctgccggtctagtaagagtctcctacatagtaatggcatcacttatttgtattggtatctgcagaaaccagggcagtctcctcagctgctcatctatcagatgtctaacagagcctcaggggtcccagacaggttcagtagcagtggatctgggacagatttcactctcaagatcagcagagtggaggctgaagatgtgggagtttattactgtgctcagaacctagagcttccgcggacgttcggtcagggcaccaagcttgagatgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 41 - protein, 239 aa, Homo sapiens
MRFSAQLLGLLVLWIPGSTADIVMTQSPLSNPVTPGESGSISCRSSKSLLHSNGITYLYWYLQKPGQSPQLLIYQMSNRASGVPDRFSSSGSGTDFTLKISRVEAEDVGVYYCAQNLELPRTFGQGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - DNA, 720 bp, Homo sapiens
  misc_feature: P1P3= Human Engineered (low risk) ING1 light Chain with one moderate risk proline change; proline at position 8 (P1) 18 (P3)
atgaggttctctgctcagcttctggggctgcttgtgctctggatccctggatccactgcagacatcgtgatgacccagtctccactctccaatccagtcactctgggagagccgggttccatctcctgccggtctagtaagagtctcctacatagtaatggcatcacttatttgtattggtatctgcagaaaccagggcagtctcctcagctgctcatctatcagatgtctaacagagcctcaggggtcccagacaggttcagtagcagtggatctgggacagatttcactctcaagatcagcagagtggaggctgaagatgtgggagtttattactgtgctcagaacctagagcttccgcggacgttcggtcagggcaccaagcttgagatgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 43 - protein, 239 aa, Homo sapiens
MRFSAQLLGLLVLWIPGSTADIVMTQSPLSNPVTLGEPGSISCRSSKSLLHSNGITYLYWYLQKPGQSPQLLIYQMSNRASGVPDRFSSSGSGTDFTLKISRVEAEDVGVYYCAQNLELPRTFGQGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - DNA, 720 bp, Homo sapiens
  misc_feature: P2P3=Human Engineered (low risk) ING1 light Chain with one moderate risk proline change; proline at position 8 (P1) 18 (P3)
atgaggttctctgctcagcttctggggctgcttgtgctctggatccctggatccactgcagacatcgtgatgacccagtctgcactctccaatccagtcactcctggagagccgggttccatctcctgccggtctagtaagagtctcctacatagtaatggcatcacttatttgtattggtatctgcagaaaccagggcagtctcctcagctgctcatctatcagatgtctaacagagcctcaggggtcccagacaggttcagtagcagtggatctgggacagatttcactctcaagatcagcagagtggaggctgaagatgtgggagtttattactgtgctcagaacctagagcttccgcggacgttcggtcagggcaccaagcttgagatgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 45 - protein, 239 aa, Homo sapiens
MRFSAQLLGLLVLWIPGSTADIVMTQSALSNPVTPGEPGSISCRSSKSLLHSNGITYLYWYLQKPGQSPQLLIYQMSNRASGVPDRFSSSGSGTDFTLKISRVEAEDVGVYYCAQNLELPRTFGQGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - DNA, 85 bp, Homo Sapiens
  misc_feature: P1 Oligo Human Engineered ING-1 with proline oligos
actcttactagaccggcaggagatggaacctgactctcccagagtgactggattggagagtggagactgggtcatcacgatgtct

SEQ ID NO: 47 - DNA, 85 bp, Homo Sapiens
  misc_feature: P2 Oligo Human Engineered ING-1 with proline oligos
actcttactagaccggcaggagatggaacctgactctccaggagtgactggattggagagtgcagactgggtcatcacgatgtct

SEQ ID NO: 48 - DNA, 85 bp, Homo Sapiens
  misc_feature: P3 Oligo Human Engineered ING-1 with proline oligos
actcttactagaccggcaggagatggaacccggctctcccagagtgactggattggagagtgcagactgggtcatcacgatgtct

SEQ ID NO: 49 - DNA, 85 bp, Homo Sapiens
  misc_feature: P1P2 Oligo Human Engineered ING-1 with proline oligos
actcttactagaccggcaggagatggaacccggctctccaggagtgactggattggagagtgcagactgggtcatcacgatgtct

SEQ ID NO: 50 - DNA, 85 bp, Homo Sapiens
  misc_feature: P1P3 Oligo Human Engineered ING-1 with proline oligos
actcttactagaccggcaggagatggaacccggctctcccagagtgactggattggagagtggagactgggtcatcacgatgtct

SEQ ID NO: 51 - DNA, 85 bp, Homo Sapiens; identical to SEQ ID NO: 49
  misc_feature: P2P3 Oligo Human Engineered ING-1 with proline oligos

SEQ ID NO: 52 - DNA, 19 bp, Homo Sapiens
  misc_feature: Reverse Primer KBsr ING-1 Light Chain
cttactagaccggcaggag

SEQ ID NO: 53 - DNA, 798 bp, Homo sapiens
  misc_feature: EpCam truncated sequence
atggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaataa

SEQ ID NO: 54 - protein, 265 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSTCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLK

SEQ ID NO: 55 - DNA, 945 bp, Homo sapiens
  misc_feature: Full-Length EpCam
atggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgcataa

SEQ ID NO: 56 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSTCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 57 - DNA, 26 bp, Homo sapiens
  misc_feature: Forward Primer (for both soluble and full length Ep-CAM)  EC-1
ttatgtcgacagcatggcgcccccgc

SEQ ID NO: 58 - DNA, 31 bp, Homo sapiens
  misc_feature: Ep-CAM Reverse Primer (for soluble Ep-CAM) EC-2
gagttacgtcccagattttattgggccccct

SEQ ID NO: 59 - DNA, 30 bp, Homo sapiens
  misc_feature: Ep-CAM Reverse Primer (for full-length Ep-CAM) EC-3
gtatcccttgagttacgtattgagctcgtt